SEQUENCE LISTING (41 sequences)

SEQ ID NO: 1 - protein, 358 aa, Unknown
  Description of Unknown Diadenylate cyclase sequence
MHAVTRPTLREAVARLAPGTGLRDGLERILRGRTGALIVLGHDENVEAICDGGFSLDVRYAATRLRELCKMDGAVVLSTDGSRIVRANVQLVPDPSIPTDESGTRHRSAERAAIQTGYPVISVSHSMNIVTVYVRGERHVLTDSATILSRANQAIATLERYKTRLDEVSRQLSRAEIEDFVTLRDVMTVVQRLELVRRIGLVIDYDVVELGTDGRQLRLQLDELLGGNDTARELIVRDYHANPEPPSTGQINATLDELDALSDGDLLDFTALAKVFGYPTTTEAQDSTLSPRGYRAMAGIPRLQFAHADLLVRAFGTLQGLLAASAGDLQSVDGIGAMWARHVREGLSQLAESTISDQ

SEQ ID NO: 2 - DNA, 1077 bp, Unknown
  Description of Unknown Diadenylate cyclase sequence
atgcacgctgtgactcgtccgaccctgcgtgaggctgtcgcccgcctagccccgggcactgggctgcgggacggcctggagcgtatcctgcgcggccgcactggtgccctgatcgtgctgggccatgacgagaatgtcgaggccatctgcgatggtggcttctccctcgatgtccgctatgcagcaacccggctacgcgagctgtgcaagatggacggcgccgtggtgctgtccaccgacggcagccgcatcgtgcgggccaacgtgcaactggtaccggatccgtcgatccccaccgacgaatcggggacccggcaccgctcggccgagcgggccgcgatccagaccggttacccggtgatctcagtgagccactcgatgaacatcgtgaccgtctacgtccgcggggaacgtcacgtattgaccgactcggcaaccatcctgtcgcgggccaaccaggccatcgcaaccctggagcggtacaaaaccaggctcgacgaggtcagccggcaactgtccagggcagaaatcgaggacttcgtcacgctgcgcgatgtgatgacggtggtgcaacgcctcgagctggtccggcgaatcgggctggtgatcgactacgacgtggtcgaactcggcactgatggtcgtcagctgcggctgcagctcgacgagttgctcggcggcaacgacaccgcccgggaattgatcgtgcgcgattaccacgccaacccggaaccaccgtccacggggcaaatcaatgccaccctggacgaactggacgccctgtcggacggcgacctcctcgatttcaccgcgctggcaaaggttttcggatatccgacgaccacggaagcgcaggattcggcgctgagcccgcgtggctaccgcgcgatggccggtatcccccggctccagttcgcccatgccgacctgctggtccgggcgttcggaacgttgcagggtctgctggcggccagcgccggcgatctgcaatcagtggacggcatcggcgccatgtgggcccgtcatgtgcgcgatgggttgtcacagctggcggaatcgaccatcagcgatcaataa

SEQ ID NO: 3 - DNA, 7742 bp, Artificial Sequence
  Description of Artificial Sequence Synthetic polynucleotide
ggatccttctagaattccggaattgcactcgccttaggggagtgctaaaaatgatcctggcactcgcgatcagcgagtgccaggtcgggacggtgagacccagccagcaagctgtggtcgtccgtcgcgggcactgcacccggccagcgtaagtaatgggggttgtcggcacccggtgacctagacacatgcatgcatgcttaattaattaagcgatatccggaggaatcacttccatatgatgcacgctgtgactcgtccgaccctgcgtgaggctgtcgcccgcctagccccgggcactgggctgcgggacggcctggagcgtatcctgcgcggccgcactggtgccctgatcgtgctgggccatgacgagaatgtcgaggccatctgcgatggtggcttctccctcgatgtccgctatgcagcaacccggctacgcgagctgtgcaagatggacggcgccgtggtgctgtccaccgacggcagccgcatcgtgcgggccaacgtgcaactggtaccggatccgtcgatccccaccgacgaatcggggacccggcaccgctcggccgagcgggccgcgatccagaccggttacccggtgatctcagtgagccactcgatgaacatcgtgaccgtctacgtccgcggggaacgtcacgtattgaccgactcggcaaccatcctgtcgcgggccaaccaggccatcgcaaccctggagcggtacaaaaccaggctcgacgaggtcagccggcaactgtccagggcagaaatcgaggacttcgtcacgctgcgcgatgtgatgacggtggtgcaacgcctcgagctggtccggcgaatcgggctggtgatcgactacgacgtggtcgaactcggcactgatggtcgtcagctgcggctgcagctcgacgagttgctcggcggcaacgacaccgcccgggaattgatcgtgcgcgattaccacgccaacccggaaccaccgtccacggggcaaatcaatgccaccctggacgaactggacgccctgtcggacggcgacctcctcgatttcaccgcgctggcaaaggttttcggatatccgacgaccacggaagcgcaggattcgacgctgagcccgcgtggctaccgcgcgatggccggtatcccccggctccagttcgcccatgccgacctgctggtccgggcgttcggaacgttgcagggtctgctggcggccagcgccggcgatctgcaatcagtggacggcatcggcgccatgtgggcccgtcatgtgcgcgaggggttgtcacagctggcggaatcgaccatcagcgatcaataaacgcgttctggcgtaatagcgaagaggcccgcaccgatcgcccttcccaacagttgcgcagcctgaatggcgaatggcgctttgcctggtttccggtcgaagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcggggatccgctcggaggcgcggtcgcggcgcggctgtggcatgtcggggcgtgccgctcccccggcgccgcccatcggcccgcccattggcattccgcccatgccgcccatcattcctgtggagccagaactgatccagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccagggaattcccatcgagccgagaacgttatcgaagttggtcatgtgtaatcccctcgtttgaactttggattaagcgtagatacacccttggacaagccagttggattcggagacaagcaaattcagccttaaaaagggcgaggccctgcggtggtggaacaccgcagggcctctaaccgctcgacgcgctgcaccaaccagcccgcgaacggctggcagccagcgtaaggcgcggctcatcgggcggcgttcgccacgatgtcctgcacttcgagccaagcctcgaacacctgctggtgtgcacgactcacccggttgttgacaccgcgcgcggccgtgcgggctcggtggggcggctctgtcgcccttgccagcgtgagtagcgcgtacctcacctcgcccaacaggtcgcacacagccgattcgtacgccataaagccaggtgagcccaccagctccgtaagttcgggcgctgtgtggctcgtacccgcgcattcaggcggcagggggtctaacgggtctaaggcggcgtgtacgcggccacagcggctctcagcggcccggaaacgtcctcgaaacgacgcatgtgttcctcctggttggtacaggtggttgggggtgctcggctgtcgcggttgttccaccaccagggctcgacgggagagcgggggagtgtgcagttgtggggtggcccctcagcgaaatatctgacttggagctcgtgtcggaccatacaccggtgattaatcgtggtctactaccaagcgtgagccacgtcgccgacgaatttgagcagctctggctgccgtactggccgctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcggccgtcgacggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcgtcccgccctcatgcggatctacctgccgacccggaacgtggacggactcggccgcgcgatctatgccgagtgccacgcgcgaaacgccgaattcccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccagtcggccatctcgcggaagggcgcagcagcgcgcacggcggcgagcacagttgcgcggcgcgcaaagtccgcgtcagccatggaggcattgctatgagcgacggctacagcgacggctacagcgacggctacaaccggcagccgactgtccgcaaaaagccgtgacgcgccgaaggcgctcgaatcaccggactatccgaacgccacgtcgtccggctcgtggcgcaggaacgcagcgagtggctcgccgagcaggctgcacgcgcgcgaagcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggacgggtgtcgcgggggttccgtggggggttccgttgcaacgggtcggacaggtaaaagtcctggtagacgctagttttctggtttgggccatgcctgtctcgttgcgtgtttcgttgcgccgttttgaataccagccagacgagacggggttctacgaatcttggtcgataccaagccatttccgctgaatatcggggagctcaccgccagaatcggtggttgtggtgatgtacgtggcgaactccgttgtagtgcctgtggtggcatccgtggccactctcgttgcacggttcgttgtgccgttacaggccccgttgacagctcaccgaacgtagttaaaacatgctggtcaaactaggtttaccaacgatacgagtcagctcatctagggccagttctaggcgttgttcgttgcgcggttcgttgcgcatgtttcgtgtggttgctagatggctccgcaaccacacgcttcgaggttgagtgcttccagcacgggcgcgatccagaagaacttcgtcgtgcgactgtcctcgttgggatctagcccgcctaatgagcgggctttttttt

SEQ ID NO: 4 - protein, 623 aa, Unknown
  Description of Unknown Bifunctional diguanylate cyclase/phosphodiesterase sequence
MCNDTATPQLEELVTTVANQLMTVDAATSAEVSQRVLAYLVEQLGVDVSFLRHNDRDRRATRLVAEWPPRLNIPDPDPLRLIYFADADPVFALCEHAKEPLVFRPEPATEDYQRLIEEARGVPVTSAAAVPLVSGEITTGLLGFIKFGDRKWHEAELNALMTIATLFAQVQARVAAEARLRYLADHDDLTGLHNRRALLQHLDQRLAPGQPGPVAALFLDLDRLKAINDYLGHAAGDQFIHVFAQRIGDALVGESLIARLGGDEFVLIPASPMSADAAQPLAERLRDQLKDHVAIGGEVLTRTVSIGVASGTPGQHTPSDLLRRADQAALAAKHAGGDSVAIFTADMSVSGELRNDIELHLRRGIESDALRLVYLPEVDLRTGDIVGTEALVRWQHPTRGLLAPGCFIPVAESINLAGELDRWVLRRACNEFSEWQSAGLGHDALLRINVSAGQLVTGGFVDFVADTIGQHGLDASSVCLEITENVVVQDLHTARATLARLKEVGVHIAIDDFGTGYSAISLLQTLPIDTLKIDKTFVRQLGTNTSDLVIVRGIMTLAEGFQLDVVAEGVETEAAARILLDQRCYRAQGFLFSRPVPGEAMRHMLSARRLPPTCIPATDPALS

SEQ ID NO: 5 - DNA, 1872 bp, Unknown
  Description of Unknown Bifunctional diguanylate cyclase/phosphodiesterase sequence
atgtgcaacgacaccgcgacgccgcagcttgaggagctcgtcaccaccgtagccaaccagctcatgacagtcgacgctgccacgtcagccgaagtcagtcagcgcgttttggcctatctagtggaacagctgggcgtagatgtcagctttttgcgtcataacgatcgcgacaggcgcgcgacgaggctggtggccgaatggccacctcgcctcaacataccggaccccgatccgctcaggctgatctacttcgctgatgccgacccggtgtttgcgctatgcgaacacgccaaagagcctctcgtgttccggcccgagccggccaccgaggactatcaacgcctcatcgaagaagcccgcggggttccggtaacgtcggctgccgccgtgccgctggtatctggcgagatcaccactggactgctggggttcatcaagttcggtgatcggaaatggcacgaggccgagcttaacgccctcatgaccatcgctacactcttcgcccaggtgcaggctcgcgtcgccgccgaggcgcggcttcgctatctggccgaccatgacgatctgaccggactgcataaccgtcgcgcgttgctgcagcacctggaccaaagactggcccccggacaacctggcccggtcgcggcgctatttctcgacttggaccgcctcaaggccatcaacgactacctgggccacgccgccggtgaccagttcatccatgtgttcgcccaacggatcggtgacgcactcgttggcgagagcctgatcgcccgactcggcggcgacgaattcgtcctcatacccgcatctccaatgagtgccgatgccgctcaaccgctcgccgaacgtcttcgcgaccagctcaaggaccacgtcgctatcggcggtgaggtgctcacccgcaccgtcagtatcggtgtcgcctcagggactcccggacagcacacaccgtcggacctcctgcgccgagccgaccaagccgctctggcagccaaacacgccggcggagatagcgtcgcgattttcaccgcggacatgtcggtcagcggcgaactgcgcaacgatattgaactacaccttcgacgtggtatcgaatccgacgcccttcgcctggtctacctacccgaggtcgacctacggaccggcgacattgtcgggaccgaggcattggtccggtggcagcaccccacccgtgggctgctggcaccgggctgcttcatccctgtggccgaatccatcaaccttgcaggcgaattggatagatgggtgctgcggagggcctgcaatgaattctccgagtggcagtcagccggtttgggccacgacgcgctgctgcgtatcaacgtctcagctggacagctggtgacgggcgggtttgttgacttcgtcgcagacacgatcggccagcacggtctggacgcctcgtccgtgtgtttggaaatcaccgaaaacgttgtggtgcaagacctacataccgccagagccaccctggctcgactcaaagaagtcggcgttcacatcgctatcgacgatttcggcaccggctatagcgccatatcactgttgcagacgctaccgatcgacacgctcaagatcgacaaaacattcgtgcggcaactcggaaccaacactagcgatctggtcattgtgcgcggcatcatgacactcgccgaaggcttccaactcgatgtagtagccgaaggcgtcgagaccgaggctgccgccagaattctattggatcagcgctgttaccgtgcgcaaggcttcttgttctcccggcctgtccccggggaggccatgcggcacatgttgtccgcacgacgactaccgccgacctgcatacctgcaactgacccggcgttatcttga

SEQ ID NO: 6 - protein, 353 aa, Artificial Sequence
  Description of Artificial Sequence Synthetic polypeptide
MCNDTATPQLEELVTTVANQLMTVDAATSAEVSQRVLAYLVEQLGVDVSFLRHNDRDRRATRLVAEWPPRLNIPDPDPLRLIYFADADPVFALCEHAKEPLVFRPEPATEDYQRLIEEARGVPVTSAAAVPLVSGEITTGLLGFIKFGDRKWHEAELNALMTIATLFAQVQARVAAEARLRYLADHDDLTGLHNRRALLQHLDQRLAPGQPGPVAALFLDLDRLKAINDYLGHAAGDQFIHVFAQRIGDALVGESLIARLGGDEFVLIPASPMSADAAQPLAERLRDQLKDHVAIGGEVLTRTVSIGVASGTPGQHTPSDLLRRADQAALAAKHAGGDSVAIFTADMSVSGEL

SEQ ID NO: 7 - DNA, 1059 bp, Artificial Sequence
  Description of Artificial Sequence Synthetic polynucleotide
atgtgcaacgacaccgcgacgccgcagcttgaggagctcgtcaccaccgtagccaaccagctcatgacagtcgacgctgccacgtcagccgaagtcagtcagcgcgttttggcctatctagtggaacagctgggcgtagatgtcagctttttgcgtcataacgatcgcgacaggcgcgcgacgaggctggtggccgaatggccacctcgcctcaacataccggaccccgatccgctcaggctgatctacttcgctgatgccgacccggtgtttgcgctatgcgaacacgccaaagagcctctcgtgttccggcccgagccggccaccgaggactatcaacgcctcatcgaagaagcccgcggggttccggtaacgtcggctgccgccgtgccgctggtatctggcgagatcaccactggactgctggggttcatcaagttcggtgatcggaaatggcacgaggccgagcttaacgccctcatgaccatcgctacactcttcgcccaggtgcaggctcgcgtcgccgccgaggcgcggcttcgctatctggccgaccatgacgatctgaccggactgcataaccgtcgcgcgttgctgcagcacctggaccaaagactggcccccggacaacctggcccggtcgcggcgctatttctcgacttggaccgcctcaaggccatcaacgactacctgggccacgccgccggtgaccagttcatccatgtgttcgcccaacggatcggtgacgcactcgttggcgagagcctgatcgcccgactcggcggcgacgaattcgtcctcatacccgcatctccaatgagtgccgatgccgctcaaccgctcgccgaacgtcttcgcgaccagctcaaggaccacgtcgctatcggcggtgaggtgctcacccgcaccgtcagtatcggtgtcgcctcagggactcccggacagcacacaccgtcggacctcctgcgccgagccgaccaagccgctctggcagccaaacacgccggcggagatagcgtcgcgattttcaccgcggacatgtcggtcagcggcgaactg

SEQ ID NO: 8 - protein, 436 aa, Vibrio cholera
MRMTWNFHQYYTNRNDGLMGKLVLTDEEKNNLKALRKIIRLRTRDVFEEAKGIAKAVKKSALTFEIIQEKVSTTQIKHLSDSEQREVAKLIYEMDDDARDEFLGLTPRFWTQGSFQYDTLNRPFQPGQEMDIDDGTYMPMPIFESEPKIGHSLLILLVDASLKSLVAENHGWKFEAKQTCGRIKIEAEKTHIDVPMYAIPKDEFQKKQIALEANRSFVKGAIFESYVADSITDDSETYELDSENVNLALREGDRKWINSDPKIVEDWFNDSCIRIGKHLRKVCRFMKAWRDAQWDVGGPSSISLMAATVNILDSVAHDASDLGETMKIIAKHLPSEFARGVESPDSTDEKPLFPPSYKHGPREMDIMSKLERLPEILSSAESADSKSEALKKINMAFGNRVTNSELIVLAKALPAFAQEPSSASKPEKISSTMVSG

SEQ ID NO: 9 - DNA, 1311 bp, Vibrio cholera
gtgagaatgacttggaactttcaccagtactacacaaaccgaaatgatggcttgatgggcaagctagttcttacagacgaggagaagaacaatctaaaggcattgcgtaagatcatccgcttaagaacacgagatgtatttgaagaagctaagggtattgccaaggctgtgaaaaaaagtgctcttacgtttgaaattattcaggaaaaggtgtcaacgacccaaattaagcacctttctgacagcgaacaacgagaagtggctaagcttatttacgagatggatgatgatgctcgtgatgagtttttgggattgacacctcgcttttggactcagggaagctttcagtatgacacgctgaatcgcccgtttcagcctggtcaagaaatggatattgatgatggaacctatatgccaatgcctatttttgagtcagagcctaagattggtcattctttactaattcttcttgttgacgcgtcacttaagtcacttgtagctgaaaatcatggctggaaatttgaagctaagcagacttgtgggaggattaagattgaggcagagaaaacacatattgatgtaccaatgtatgcaatccctaaagatgagttccagaaaaagcaaatagctttagaagcaaatagatcatttgttaaaggtgccatttttgaatcatatgttgcagattcaattactgacgatagtgaaacttatgaattagattcagaaaacgtaaaccttgctcttcgtgaaggtgatcggaagtggatcaatagcgaccccaaaatagttgaagattggttcaacgatagttgtatacgtattggtaaacatcttcgtaaggtttgtcgctttatgaaagcgtggagagatgcgcagtgggatgttggaggtccgtcatcgattagtcttatggctgcaacggtaaatattcttgatagcgttgctcatgatgctagtgatctcggagaaacaatgaagataattgctaagcatttacctagtgagtttgctaggggagtagagagccctgacagtaccgatgaaaagccactcttcccaccctcttataagcatggccctcgggagatggacattatgagcaaactagagcgtttgccagagattctgtcatctgctgagtcagctgactctaagtcagaggccttgaaaaagattaatatggcgtttgggaatcgtgttactaatagcgagcttattgttttggcaaaggctttaccggctttcgctcaagaacctagttcagcctcgaaacctgaaaaaatcagcagcacaatggtaagtggctga

SEQ ID NO: 10 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 11 - DNA, 1802 bp, Homo sapiens
agcctggggttccccttcgggtcgcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcac

SEQ ID NO: 12 - DNA, 1569 bp, Artificial Sequence
  Description of Artificial Sequence Synthetic polynucleotide
atgcaaccatggcacgggaaagccatgcagcgtgcgagcgaagccggggcgacggcccccaaggcgtcggcgcgtaacgcgcggggtgcgcccatggacccgacggagtcccccgcggcgccggaggcggccctgccgaaagcgggtaagttcggtccagcgcggaaaagcgggagccgccaaaagaagtccgcgcccgacacccaggagcgtcccccggtccgggccaccggcgcgcgtgccaaaaaagccccgcaacgggcgcaagatacgcagccaagcgatgcgacctccgcccccggggcggagggtctggagcccccggccgcccgggagccagcgctctcgcgcgcgggttcctgccgtcagcggggcgcgcggtgttccacgaaaccccgtcccccaccaggtccctgggacgtgccgtcgccgggtttgccggtgagcgcgccaatcctggtccggcgcgacgcggccccgggggcgtcgaaattgcgtgcggtgctcgagaaattgaagttgtcgcgcgacgacatctccacggccgcgggtatggtcaagggcgtggtcgatcatttgttgttgcggctcaagtgtgattcggcgttccgcggggtgggcttgctgaacacggggtcctactatgagcatgtcaaaatcagcgcccccaacgaatttgacgtgatgtttaagctggaagtgccacgtatccaattggaagagtattccaatacccgtgcgtattatttcgtcaaatttaagcgcaatccgaaggaaaatccactcagccaattcttggagggcgaaattctgtcggcctcgaaaatgctctccaaatttcgtaagattatcaaggaggagatcaacgacattaaggacacggatgtgatcatgaaacgtaaacgtggcggttcccccgcggtgacgctcctcatttcggaaaaaatttcggtggacattaccctggcgttggaatcgaagtccagctggccggcgtcgacccaggagggcctgcggattcaaaactggttgagcgccaaagtgcggaagcagctgcgtctcaaacccttttatttggtcccgaaacatgccaaagagggtaacggttttcaagaggaaacctggcgtttgagcttctcccacattgagaaggagattttgaacaaccatggtaagtccaaaacgtgctgcgagaataaggaagaaaaatgttgtcgcaaagattgtctcaaattgatgaaatatttgctggaacaactcaaagagcgttttaaggacaagaagcatctcgacaagttctcctcgtatcacgtcaagaccgccttctttcatgtctgtacgcagaacccgcaagatagccagtgggatcgcaaggacttggggttgtgttttgacaattgcgtcacctatttcttgcaatgtttgcggaccgagaaattggagaactactttattccagaattcaacttgttttcctcgaatctgattgacaaacgctccaaagagtttctgacgaagcagattgaatacgagcgtaacaatgagtttccggtctttgacgagttttga

SEQ ID NO: 13 - DNA, 10841 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagaaattccggaattgcactcgccttaggggagtgctaaaaatgatcctggcactcgcgatcagcgagtgccaggtcgggacggtgagacccagccagcaagctgtggtcgtccgtcgcgggcactgcacccggccagcgtaagtaatgggggttgtcggcacccggtgacatgcacgctgtgactcgtccgaccctgcgtgaggctgtcgcccgcctagccccgggcactgggctgcgggacggcctggagcgtatcctgcgcggccgcactggtgccctgatcgtgctgggccatgacgagaatgtcgaggccatctgcgatggtggcttctccctcgatgtccgctatgcagcaacccggctacgcgagctgtgcaagatggacggcgccgtggtgctgtccaccgacggcagccgcatcgtgcgggccaacgtgcaactggtaccggatccgtcgatccccaccgacgaatcggggacccggcaccgctcggccgagcgggccgcgatccagaccggttacccggtgatctcagtgagccactcgatgaacatcgtgaccgtctacgtccgcggggaacgtcacgtattgaccgactcggcaaccatcctgtcgcgggccaaccaggccatcgcaaccctggagcggtacaaaaccaggctcgacgaggtcagccggcaactgtccagggcagaaatcgaggacttcgtcacgctgcgcgatgtgatgacggtggtgcaacgcctcgagctggtccggcgaatcgggctggtgatcgactacgacgtggtcgaactcggcactgatggtcgtcagctgcggctgcagctcgacgagttgctcggcggcaacgacaccgcccgggaattgatcgtgcgcgattaccacgccaacccggaaccaccgtccacggggcaaatcaatgccaccctggacgaactggacgccctgtcggacggcgacctcctcgatttcaccgcgctggcaaaggttttcggatatccgacgaccacggaagcgcaggattcgacgctgagcccgcgtggctaccgcgcgatggccggtatcccccggctccagttcgcccatgccgacctgctggtccgggcgttcggaacgttgcagggtctgctggcggccagcgccggcgatctgcaatcagtggacggcatcggcgccatgtgggcccgtcatgtgcgcgaggggttgtcacagctggcggaatcgaccatcagcgatcaataagagcacatcgatatgcaaccatggcacgggaaagccatgcagcgtgcgagcgaagccggggcgacggcccccaaggcgtcggcgcgtaacgcgcggggtgcgcccatggacccgacggagtcccccgcggcgccggaggcggccctgccgaaagcgggtaagttcggtccagcgcggaaaagcgggagccgccaaaagaagtccgcgcccgacacccaggagcgtcccccggtccgggccaccggcgcgcgtgccaaaaaagccccgcaacgggcgcaagatacgcagccaagcgatgcgacctccgcccccggggcggagggtctggagcccccggccgcccgggagccagcgctctcgcgcgcgggttcctgccgtcagcggggcgcgcggtgttccacgaaaccccgtcccccaccaggtccctgggacgtgccgtcgccgggtttgccggtgagcgcgccaatcctggtccggcgcgacgcggccccgggggcgtcgaaattgcgtgcggtgctcgagaaattgaagttgtcgcgcgacgacatctccacggccgcgggtatggtcaagggcgtggtcgatcatttgttgttgcggctcaagtgtgattcggcgttccgcggggtgggcttgctgaacacggggtcctactatgagcatgtcaaaatcagcgcccccaacgaatttgacgtgatgtttaagctggaagtgccacgtatccaattggaagagtattccaatacccgtgcgtattatttcgtcaaatttaagcgcaatccgaaggaaaatccactcagccaattcttggagggcgaaattctgtcggcctcgaaaatgctctccaaatttcgtaagattatcaaggaggagatcaacgacattaaggacacggatgtgatcatgaaacgtaaacgtggcggttcccccgcggtgacgctcctcatttcggaaaaaatttcggtggacattaccctggcgttggaatcgaagtccagctggccggcgtcgacccaggagggcctgcggattcaaaactggttgagcgccaaagtgcggaagcagctgcgtctcaaacccttttatttggtcccgaaacatgccaaagagggtaacggttttcaagaggaaacctggcgtttgagcttctcccacattgagaaggagattttgaacaaccatggtaagtccaaaacgtgctgcgagaataaggaagaaaaatgttgtcgcaaagattgtctcaaattgatgaaatatttgctggaacaactcaaagagcgttttaaggacaagaagcatctcgacaagttctcctcgtatcacgtcaagaccgccttctttcatgtctgtacgcagaacccgcaagatagccagtgggatcgcaaggacttggggttgtgttttgacaattgcgtcacctatttcttgcaatgtttgcggaccgagaaattggagaactactttattccagaattcaacttgttttcctcgaatctgattgacaaacgctccaaagagtttctgacgaagcagattgaatacgagcgtaacaatgagtttccggtctttgacgagttttgaaagcttgagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagactagttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggaagggtggcgggcaggacgcccgccataaactgccaggcatcaaattaagcagaaggccatcctgacggatggccttttttctagagtcgaccaccaagggcaccatctctgcttgggccaccccgttggccgcagccagctcgctgagagccgtgaacgacagggcgaacgccagcccgccgacggcgagggttccgaccgctgcaactcccggtgcaaccttgtcccggtctattctcttcactgcaccagctccaatctggtgtgaatgcccctcgtctgttcgcgcaggcggggggctctattcgtttgtcagcatcgaaagtagccagatcagggatgcgttgcaaccgcgtatgcccaggtcagaagagtcgcacaagagttgcagacccctggaaagaaaaatggccagagggcgaaaacaccctctgaccagcggagcgggcgacgggaatcgaacccgcgtagctagtttggaagaatgggtgtctgccgaccacatatgggccggtcaagataggtttttaccccctctcggctgcatcctctaagtggaaagaaattgcaggtcgtagaagcgcgttgaagcctgagagttgcacaggagttgcaacccggtagccttgttcacgacgagaggagacctagttggcacgtcgcggatggggatcgctgaagactcagcgcagcgggaggatccaagcctcatacgtcaacccgcaggacggtgtgaggtactacgcgctgcagacctacgacaacaagatggacgccgaagcctggctcgcgggcgagaagcggctcatcgagatggagacctggacccctccacaggaccgggcgaagaaggcagccgccagcgccatcacgctggaggagtacacccggaagtggctcgtggagcgcgacctcgcagacggcaccagggatctgtacagcgggcacgcggagcgccgcatctacccggtgctaggtgaagtggcggtcacagagatgacgccagctctggtgcgtgcgtggtgggccgggatgggtaggaagcacccgactgcccgccggcatgcctacaacgtcctccgggcggtgatgaacacagcggtcgaggacaagctgatcgcagagaacccgtgccggatcgagcagaaggcagccgatgagcgcgacgtagaggcgctgacgcctgaggagctggacatcgtcgccgctgagatcttcgagcactaccggatcgcggcatacatcctggcgtggacgagcctccggttcggagagctgatcgagcttcgccgcaaggacatcgtggacgacggcatgacgatgaagctccgggtgcgccgtggcgcttcccgcgtggggaacaagatcgtcgttggcaacgccaagaccgtccggtcgaagcgtcctgtgacggttccgcctcacgtcgcggagatgatccgagcgcacatgaaggaccgtacgaagatgaacaagggccccgaggcattcctggtgaccacgacgcagggcaaccggctgtcgaagtccgcgttcaccaagtcgctgaagcgtggctacgccaagatcggtcggccggaactccgcatccacgacctccgcgctgtcggcgctacgttcgccgctcaggcaggtgcgacgaccaaggagctgatggcccgtctcggtcacacgactcctaggatggcgatgaagtaccagatggcgtctgaggcccgcgacgaggctatcgctgaggcgatgtccaagctggccaagacctcctgaaacgcaaaaagcccccctcccaaggacactgagtcctaaagaggggggtttcttgtcagtacgcgaagaaccacgcctggccgcgagcgccagcaccgccgctctgtgcggagacctgggcaccagccccgccgccgccaggagcattgccgttcccgccagctgagttctgttgtgcgccgcctatgtagagctggtcgttgtaggtccgatctccaggcgactttccggcgacgctgaggatgtcgatcacagagcctccgggaccgccggttgcggtcaaacctgaccatccgacagcggacgccgtggtgtttcctccagggcctccggccttgcctgagaatacagagccagctcccgctgcgcctccagctccgacgagcccggtgatcgtcttggtcgacctgcaggcatgcaaaagctgatccttgccgagctgggatggaagcccggccgacccaccctggaggagatgatcgaggatgccagggcctttcacgcccgccgctgctgagcgtccgccgccgggcccgcaccgccgtcggccggcccgctccgggctcgcagcagcgggcttcggcgcgggcccggggctcccgggccgccgggcggggctccgcccggcggccgccgggggccgggggcggcgccgggcggcccggggcgtcaggcgccgggggcggtgtccggcggcccccagaggaactgcgccagttcctccggatcggtgaagccggagagatccagcggggtctcctcgaacacctcgaagtcgtgcaggaaggtgaaggcgagcagttcgcgggcgaagtcctcggtccgcttccactgcgccccgtcgagcagcgcggccaggatctcgcggtcgccccggaaggcgttgagatgcagttgcaccaggctgtagcgggagtctcccgcatagacgtcggtgaagtcgacgatcccggtgacctcggtcgcggccaggtccacgaagatgttggtcccgtgcaggtcgccgtggacgaaccggggttcgcggccggccagcagcgtgtccacgtccggcagccagtcctccaggcggtccagcagccggggcgagaggtagccccacccgcggtggtcctcgacggtcgccgcgcggcgttcccgcagcagttccgggaagacctcggaatggggggtgagcacggtgttcccggtcagcggcaccctgtgcagccggccgagcacccggccgagttcgcgggccagggcgagcagcgcgttccggtcggtcgtgccgtccatcgcggaccgccaggtggtgccggtcatccggctcatcaccaggtagggccacggccaggctccggtgccgggccgcagctcgccgcggccgaggaggcggggcaccggcaccggggcgtccgccaggaccgcgtacgcctccgactccgacgcgaggctctccggaccgcaccagtgctcgccgaacagcttgatcaccgggccgggctcgccgaccagtacggggttggtgctctcgccgggcacccgcagcaccggcggcaccggcagcccgagctcctccagggctcggcgggccagcggctcccagaattcctggtcgttccgcaggctcgcgtaggaatcatccgaatcaatacggtcgagaagtaacagggattcttgtgtcacagcggacctctattcacagggtacgggccggcttaattccgcacggccggtcgcgacacggcctgtccgcaccgcggatcaggcgttgacgatgacgggctggtcggccacgtcggggacgttctcggtggtgctgcggtcgggatcgccaatctctacgggccgaccgaggcgacggtgtacgccaccgcctggttctgcgacggcgaggcgccgtcccaggccccgccgatccccgtcccccgcgtcgtcgagcgcggtgccgacgacaccgccgcgtggctcgtcacggaggccgtccccggcgtcgcggcggccgaggagtggcccgagcaccagcggttcgccgtggtcgaggcgatggcggagctggcccgcgccctccacgagctgcccgtggaggactgccccttcgaccggcgcctcgacgcggcggtcgccgaggcccggcggaacgtcgccgagggcctgtggacctcgacgacctgcaggcatgcaagctagcttttgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - protein, 316 aa, Mycobacterium bovis
MDAVGAAALLSAAARVGVVCHVHPDADTIGAGLALALVLDGCGKRVEVSFAAPATLPESLRSLPGCHLLVRPEVMRRDVDLVVTVDIPSVDRLGALGDLTDSGRELLVIDHHASNDLFGTANFIDPSADSTTTMVAEILDAWGKPIDPRVAHCIYAGLATDTGSFRWASVRGYRLAARLVEIGVDNATVSRTLMDSHPFTWLPLLSRVLGSAQLVSEAVGGRGLVYVVVDNREWVAARSEEVESIVDIVRTTQQAEVAAVFKEVEPHRWSVSMRAKTVNLAAVASGFGGGGHRLAAGYTTTGSIDDAVASLRAALG

SEQ ID NO: 15 - DNA, 951 bp, Mycobacterium bovis
gtggacgccgtcggtgccgctgcgctgttgtcggccgctgccagggtcggggtagtctgccacgtccaccccgatgccgacaccatcggcgccggattggcattggcattggtgttggacgggtgcggcaagcgggtagaggtcagctttgccgcgccggcgacactgcccgagtcgctgcgttcgctgccgggctgccatctgctggtccgccctgaggtgatgcgccgcgatgtcgatttggttgtgactgttgacattccgagtgttgatcggctcggtgctctgggcgatctaactgattccgggcgggagctcctggtaatcgaccatcacgcctccaacgacctgttcggcaccgcgaatttcattgacccgtcggcggattccaccacgacgatggttgccgagatcctcgacgcgtgggggaaaccgatagacccgcgcgtcgcgcactgcatctacgccgggttggcgaccgacacggggtcgtttcgctgggccagtgtgcgggggtatcggctggcggcgcggctggtagagatcggtgtggacaacgccaccgtcagcaggaccttgatggacagccatcccttcacctggttgccgttgctatcgcgggtgttgggttcggcgcagctggtgtccgaggcggtcggtggccgcgggctggtttacgtcgtcgtcgacaaccgggagtgggtcgctgcgcgctcggaggaagtggaaagcatcgtcgacatcgtccgcaccacgcaacaagccgaggtcgcggcggtgttcaaggaggtcgaaccgcatcggtggtcggtgtcgatgcgggctaagaccgtgaatttggccgcggttgcctctgggttcggtggcggtggtcaccggctggccgcggggtatacgaccaccggctcgatcgacgacgctgtggcgtcgttgcgcgcggcgcttggttag

SEQ ID NO: 16 - protein, 336 aa, Mycobacterium tuberculosis
MTTIDPRSELVDGRRRAGARVDAVGAAALLSAAARVGVVCHVHPDADTIGAGLALALVLDGCGKRVEVSFAAPATLPESLRSLPGCHLLVRPEVMRRDVDLVVTVDIPSVDRLGALGDLTDSGRELLVIDHHASNDLFGTANFIDPSADSTTTMVAEILDAWGKPIDPRVAHCIYAGLATDTGSFRWASVRGYRLAARLVEIGVDNATVSRTLMDSHPFTWLPLLSRVLGSAQLVSEAVGGRGLVYVVVDNREWVAARSEEVESIVDIVRTTQQAEVAAVFKEVEPHRWSVSMRAKTVNLAAVASGFGGGGHRLAAGYTTTGSIDDAVASLRAALG

SEQ ID NO: 17 - protein, 288 aa, Unknown
  Description of Unknown Diguanylate phosphodiesterase sequence
MIDYEEMFRGAMQARAMVANPDQWADSDRDQVNTRHYLSTSMRVALDRGEFFLVYQPIIRLADNRIIGAEALLRWEHPTLGTLLPGRFIDRAENNGLMVPLTAFVLEQACRHVRSWRDHSTDPQPFVSVNVSASTICDPGFLVLVEGVLGETGLPAHALQLELAEDARLSRDEKAVTRLQELSALGVGIAIDDFGIGFSSLAYLPRLPVDVVKLGGKFIECLDGDIQARLANEQITRAMIDLGDKLGITVTAKLVESPSQAARLRAFGCKAAQGWHFAKALPVDFFRE

SEQ ID NO: 18 - DNA, 867 bp, Unknown
  Description of Unknown Diguanylate phosphodiesterase sequence
ttgatcgactacgaagagatgtttaggggcgcgatgcaagcgcgagcgatggtagccaatcctgaccaatgggcggactccgaccgcgaccaggtcaacactcgccattatctgtccacttcgatgcgcgtggcactggatcgcggtgaattcttcctcgtctaccagccaatcatccggcttgccgacaaccgcatcatcggcgccgaggccctgctgcgctgggaacacccgacgttgggcacgctactcccgggccggttcatcgaccgtgccgagaacaacggactgatggtgccgctcacggccttcgtgctcgagcaggcctgccgccacgtccgcagttggcgtgaccacagcaccgacccgcaaccgtttgtcagcgtcaacgtctccgccagcaccatctgcgatcccggcttcctggtgctggtcgaaggtgtgctcggcgaaaccggcctgcccgcccatgccctgcagctcgaactggccgaggacgcgcgccttagcagagacgagaaggcggtgaccaggctacaagaattgtccgctctcggcgtcggcatcgccatcgacgacttcggcattggattctccagcctcgcctaccttccccgcctccccgtcgacgtggtcaaactcgggggaaagttcatcgagtgcctcgatggcgacattcaagctcggctggccaacgaacagatcacccgggcaatgatcgaccttggcgacaagctcggtatcaccgtcactgcaaagctagtcgaaagccccagccaagccgcccggttgcgcgccttcggctgtaaagccgcacaaggctggcactttgccaaggcactgccggtcgactttttcagagagtag

SEQ ID NO: 19 - protein, 307 aa, Mycobacterium tuberculosis
MDRCCQRATAFACALRPTKLIDYEEMFRGAMQARAMVANPDQWADSDRDQVNTRHYLSTSMRVALDRGEFFLVYQPIIRLADNRIIGAEALLRWEHPTLGTLLPGRFIDRAENNGLMVPLTAFVLEQACRHVRSWRDHSTDPQPFVSVNVSASTICDPGFLVLVEGVLGETGLPAHALQLELAEDARLSRDEKAVTRLQELSALGVGIAIDDFGIGFSSLAYLPRLPVDVVKLGGKFIECLDGDIQARLANEQITRAMIDLGDKLGITVTAKLVETPSQAARLRAFGCKAAQGWHFAKALPVDFFRE

SEQ ID NO: 20 - DNA, 1350 bp, Mycobacterium bovis
atgacgattcctgcgttccatcccggtgaactcaatgtgtactcggcaccgggggatgtcgccgatgtcagtcgcgcactgcgactcaccggccggcgagtgatgttggtgcctactatgggtgcgctgcacgaaggccacctcgcgttggtgcgtgcggccaagcgggtgcccggatcggtcgtcgtcgtgtcgatcttcgtcaacccgatgcaattcggtgccggggaagatctcgacgcctatccccgcaccccggacgacgacctggcgcaactgcgggccgaaggcgtggaaatcgctttcacgccaactaccgcggcgatgtatcccgacggcctgcgcaccaccgtgcaacccggtccgttggccgccgaactcgagggcggcccgcggccaacccatttcgccggcgtgctgacggtcgtgctaaagctgctgcagatcgtgcgcccggatcgggtgttcttcggtgagaaggactaccagcagctggtgctgatccggcagctggtcgcggacttcaacctcgatgtcgcggtggtcggcgtgccgaccgtgcgcgaagccgacgggctggcgatgtcgtcgcgcaaccgctacctggacccggcccagcgtgcggcggccgtcgcgctctcggcggcgctaacggccgcagcgcatgcggcaacggctggcgcgcaggccgcgctggatgccgcccgtgcggtgctcgacgctgcacccggcgtggcggtcgactacctggagctgcgcgatatcgggcttggcccgatgccgctcaacggttccggtcggctgctggttgctgcccggcttggcaccaccaggctgctggacaacattgcgattgaaatcggaactttcgccggcaccgaccgcccggacggataccgggcaatcctcgaatcacattggagaaactgatgttacggacgatgctgaagtcgaagatccaccgcgccacggtgacctgcgccgacctgcactacgtcggctcggtgaccatcgatgccgacttgatggacgccgccgacctgctggaaggcgaacaggtaaccatcgtcgatatcgacaacggtgctcgactggtcacctacgcgatcaccggcgaacgcggcagtggtgtgattggcatcaacggtgccgccgcgcacttggtgcatccgggggatctggtgattctgattgcgtacgcgacgatggacgacgcccgggcccgcacataccagccgcggatcgtgtttgtcgacgcttacaacaaaccgatcgacatgggccacgatccggcatttgtgcccgaaaacgcgggcgagctgctagacccccggctcggtgtgggatagc

SEQ ID NO: 21 - protein, 309 aa, Mycobacterium bovis
MTIPAFHPGELNVYSAPGDVADVSRALRLTGRRVMLVPTMGALHEGHLALVRAAKRVPGSVVVVSIFVNPMQFGAGEDLDAYPRTPDDDLAQLRAEGVEIAFTPTTAAMYPDGLRTTVQPGPLAAELEGGPRPTHFAGVLTVVLKLLQIVRPDRVFFGEKDYQQLVLIRQLVADFNLDVAVVGVPTVREADGLAMSSRNRYLDPAQRAAAVALSAALTAAAHAATAGAQAALDAARAVLDAAPGVAVDYLELRDIGLGPMPLNGSGRLLVAARLGTTRLLDNIAIEIGTFAGTDRPDGYRAILESHWRN

SEQ ID NO: 22 - protein, 139 aa, Mycobacterium bovis
MLRTMLKSKIHRATVTCADLHYVGSVTIDADLMDAADLLEGEQVTIVDIDNGARLVTYAITGERGSGVIGINGAAAHLVHPGDLVILIAYATMDDARARTYQPRIVFVDAYNKPIDMGHDPAFVPENAGELLDPRLGVG

SEQ ID NO: 23 - DNA, 2501 bp, Mycobacterium bovis
atgacgattcctgcgttccatcccggtgaactcaatgtgtactcggcaccgggggatgtcgccgatgtcagtcgcgcactgcgactcaccggccggcgagtgatgttggtgcctactatgggtgcgctgcacgaaggccacctcgcgttggtgcgtgcggccaagcgggtgcccggatcggtcgtcgtcgtgtcgatcttcgtcaacccgatgcaattcggtgccggggaagatctcgacgcctatccccgcaccccggacgacgacctggcgcaactgcgggccgaaggcgtggaaatcgctttcacgccaactaccgcggcgatgtatcccgacggcctgcgcaccaccgtgcaacccggtccgttggccgccgaactcgagggcggcccgcggccaacccatttcgccggcgtgctgacggtcgtgctaaagctgctgcagatcgtgcgcccggatcgggtgttcttcggtgagaaggactaccagcagctggtgctgatccggcagctggtcgcggacttcaacctcgatgtcgcggtggtcggcgtgccgaccgtgcgcgaagccgacgggctggcgatgtcgtcgcgcaaccgctacctggacccggcccagcgtgcggcggccgtcgcgctctcggcggcgctaacggccgcagcgcatgcggcaacggctggcgcgcaggccgcgctggatgccgcccgtgcggtgctcgacgctgcacccggcgtggcgtggcggtcgactacctggagctgcgcgatatcgggcttggcccgatgccgctcaacggttccggtcggctgctggttgctgcccggcttggcaccaccaggctgctggacaacattgcgattgaaatcggaactttcgccggcaccgaccgcccggacggataccgggcaatcctcgaatcacattggagaaactgatgttacggacgatgctgaagtcgaagatccaccgcgccacggtgacctgcgccgacctgcactacgtcggctcggtgaccatcgatgccgacttgatggacgccgccgacctgctggaaggcgaacaggtaaccatcgtcgatatcgacaacggtgctcgactggtcacctacgcgatcaccggcgaacgcggcagtggtgtgattggcatcaacggtgccgccgcgcacttggtgcatccgggggatctggtgattctgattgcgtacgcgacgatggacgacgcccgggcccgcacataccagccgcggatcgtgtttgtcgacgcttacaacaaaccgatcgacatgggccacgatccggcatttgtgcccgaaaacgcgggcgagctgctagacccccggctcggtgtgggatagccgtgctgctggcgattgacgtccgcaacacccacaccgttgtgggcctgctgtccggaatgaaagagcacgcaaaggtcgtgcagcagtggcggatacgcaccgaatccgaagtcaccgccgacgaactggcactgacgatcgacgggctgatcggcgaggattccgagcggctcaccggtaccgccgccttgtccacggtcccgtccgtgctgcacgaggtgcggataatgctcgaccagtactggccgtcggtgccgcacgtgctgatcgagcccggagtacgcaccgggatccctttgctcgtcgacaacccgaaggaagtgggcgcagaccgcatcgtgaactgtttggccgcctatgaccggttccggaaggccgccatcgtcgttgactttggatcctcgatctgtgttgatgttgtatcggccaagggtgaatttcttggcggcgccatcgcgcccggggtgcaggtgtcttccgatgccgcggcggcccgctcggcggcattgcgccgcgttgaacttgcccgcccacgttcggtggttggcaagaacaccgtcgaatgcatgcaagccggtgcggtgttcggcttcgccgggctggtagacgggttggtaggccgcatccgcgaggacgtgtccggtttctccgtcgaccacgatgtcgcgatcgtggctaccgggcataccgcgcccctgctgctgccggaattgcacaccgtcgaccattacgaccagcacctgaccttgcagggtctgcggctggtgttcgagcgtaacctcgaagtccagcgcggccggctcaagacggcgcgctgacgtcgatgccggcatcgagtctgggtaccgggtcgcccgccgccgacaggctcgacgccacccacgagcgtcggcgtgaggtcatttaagctggcacgtcgtgagtgccgctgacacagcagaagaccttcctgagcagttccggattcgccgggacaagcgcgctcgcttgctggcccaggggcgcgatccctatcccgtcgcggtgccgcgcactcacacgttggccgaggttcgcgccgcccaccctgacttgccgatcgataccgcgaccgaagacatcgtcggcgtcgcgggccgagtgatctttgcgcgcaactcgggaa

SEQ ID NO: 24 - protein, 724 aa, Mycobacterium bovis
MTIPAFHPGELNVYSAPGDVADVSRALRLTGRRVMLVPTMGALHEGHLALVRAAKRVPGSVVVVSIFVNPMQFGAGEDLDAYPRTPDDDLAQLRAEGVEIAFTPTTAAMYPDGLRTTVQPGPLAAELEGGPRPTHFAGVLTVVLKLLQIVRPDRVFFGEKDYQQLVLIRQLVADFNLDVAVVGVPTVREADGLAMSSRNRYLDPAQRAAAVALSAALTAAAHAATAGAQAALDAARAVLDAAPGVAWRSTTWSCAISGLARCRSTVPVGCWLLPGLAPPGCWTTLRLKSELSPAPTARTDTGQSSNHIGETDVTDDAEVEDPPRHGDLRRPALRRLGDHRCRLDGRRRPAGRRTGNHRRYRQRCSTGHLRDHRRTRQWCDWHQRCRRALGASGGSGDSDCVRDDGRRPGPHIPAADRVCRRLQQTDRHGPRSGICARKRGRAARPPARCGIAVLLAIDVRNTHTVVGLLSGMKEHAKVVQQWRIRTESEVTADELALTIDGLIGEDSERLTGTAALSTVPSVLHEVRIMLDQYWPSVPHVLIEPGVRTGIPLLVDNPKEVGADRIVNCLAAYDRFRKAAIVVDFGSSICVDVVSAKGEFLGGAIAPGVQVSSDAAAARSAALRRVELARPRSVVGKNTVECMQAGAVFGFAGLVDGLVGRIREDVSGFSVDHDVAIVATGHTAPLLLPELHTVDHYDQHLTLQGLRLVFERNLEVQRGRLKTAR

SEQ ID NO: 25 - protein, 139 aa, Mycobacterium bovis; identical to SEQ ID NO: 22

SEQ ID NO: 26 - DNA, 930 bp, Mycobacterium bovis
atgacgattcctgcgttccatcccggtgaactcaatgtgtactcggcaccgggggatgtcgccgatgtcagtcgcgcactgcgactcaccggccggcgagtgatgttggtgcctactatgggtgcgctgcacgaaggccacctcgcgttggtgcgtgcggccaagcgggtgcccggatcggtcgtcgtcgtgtcgatcttcgtcaacccgatgcaattcggtgccggggaagatctcgacgcctatccccgcaccccggacgacgacctggcgcaactgcgggccgaaggcgtggaaatcgctttcacgccaactaccgcggcgatgtatcccgacggcctgcgcaccaccgtgcaacccggtccgttggccgccgaactcgagggcggcccgcggccaacccatttcgccggcgtgctgacggtcgtgctaaagctgctgcagatcgtgcgcccggatcgggtgttcttcggtgagaaggactaccagcagctggtgctgatccggcagctggtcgcggacttcaacctcgatgtcgcggtggtcggcgtgccgaccgtgcgcgaagccgacgggctggcgatgtcgtcgcgcaaccgctacctggacccggcccagcgtgcggcggccgtcgcgctctcggcggcgctaacggccgcagcgcatgcggcaacggctggcgcgcaggccgcgctggatgccgcccgtgcggtgctcgacgctgcacccggcgtggcggtcgactacctggagctgcgcgatatcgggcttggcccgatgccgctcaacggttccggtcggctgctggttgctgcccggcttggcaccaccaggctgctggacaacattgcgattgaaatcggaactttcgccggcaccgaccgcccggacggataccgggcaatcctcgaatcacattggagaaactga

SEQ ID NO: 27 - DNA, 420 bp, Mycobacterium bovis
atgttacggacgatgctgaagtcgaagatccaccgcgccacggtgacctgcgccgacctgcactacgtcggctcggtgaccatcgatgccgacttgatggacgccgccgacctgctggaaggcgaacaggtaaccatcgtcgatatcgacaacggtgctcgactggtcacctacgcgatcaccggcgaacgcggcagtggtgtgattggcatcaacggtgccgccgcgcacttggtgcatccgggggatctggtgattctgattgcgtacgcgacgatggacgacgcccgggcccgcacataccagccgcggatcgtgtttgtcgacgcttacaacaaaccgatcgacatgggccacgatccggcatttgtgcccgaaaacgcgggcgagctgctagacccccggctcggtgtgggatag

SEQ ID NO: 28 - DNA, 19 bp, Artificial Sequence
  Description of Artificial Sequence Synthetic primer
ggggatgacgattcctgcg

SEQ ID NO: 29 - DNA, 19 bp, Artificial Sequence
  Description of Artificial Sequence Synthetic primer
gggctatcccacaccgagc

SEQ ID NO: 30 - DNA, 7738 bp, Artificial Sequence
  Description of Artificial Sequence Synthetic polynucleotide
ggatccttctagaattccggaattgcactcgccttaggggagtgctaaaaatgatcctggcactcgcgatcagcgagtgccaggtcgggacggtgagacccagccagcaagctgtggtcgtccgtcgcgggcactgcacccggccagcgtaagtaatgggggttgtcggcacccggtgacctagacacatgcatgcatgcttaattaattaagcgatatccggaggaatcacttccatatgcacgctgtgactcgtccgaccctgcgtgaggctgtcgcccgcctagccccgggcactgggctgcgggacggcctggagcgtatcctgcgcggccgcactggtgccctgatcgtgctgggccatgacgagaatgtcgaggccatctgcgatggtggcttctccctcgatgtccgctatgcagcaacccggctacgcgagctgtgcaagatggacggcgccgtggtgctgtccaccgacggcagccgcatcgtgcgggccaacgtgcaactggtaccggatccgtcgatccccaccgacgaatcggggacccggcaccgctcggccgagcgggccgcgatccagaccggttacccggtgatctcagtgagccactcgatgaacatcgtgaccgtctacgtccgcggggaacgtcacgtattgaccgactcggcaaccatcctgtcgcgggccaaccaggccatcgcaaccctggagcggtacaaaaccaggctcgacgaggtcagccggcaactgtccagggcagaaatcgaggacttcgtcacgctgcgcgatgtgatgacggtggtgcaacgcctcgagctggtccggcgaatcgggctggtgatcgactacgacgtggtcgaactcggcactgatggtcgtcagctgcggctgcagctcgacgagttgctcggcggcaacgacaccgcccgggaattgatcgtgcgcgattaccacgccaacccggaaccaccgtccacggggcaaatcaatgccaccctggacgaactggacgccctgtcggacggcgacctcctcgatttcaccgcgctggcaaaggttttcggatatccgacgaccacggaagcgcaggattcgacgctgagcccgcgtggctaccgcgcgatggccggtatcccccggctccagttcgcccatgccgacctgctggtccgggcgttcggaacgttgcagggtctgctggcggccagcgccggcgatctgcaatcagtggacggcatcggcgccatgtgggcccgtcatgtgcgcgaggggttgtcacagctggcggaatcgaccatcagcgatcaataacgcgttctggcgtaatagcgaagaggcccgcaccgatcgcccttcccaacagttgcgcagcctgaatggcgaatggcgctttgcctggtttccggtcgaagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcggggatccgctcggaggcgcggtcgcggcgcggctgtggcatgtcggggcgtgccgctcccccggcgccgcccatcggcccgcccattggcattccgcccatgccgcccatcattcctgtggagccagaactgatccagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccagggaattcccatcgagccgagaacgttatcgaagttggtcatgtgtaatcccctcgtttgaactttggattaagcgtagatacacccttggacaagccagttggattcggagacaagcaaattcagccttaaaaagggcgaggccctgcggtggtggaacaccgcagggcctctaaccgctcgacgcgctgcaccaaccagcccgcgaacggctggcagccagcgtaaggcgcggctcatcgggcggcgttcgccacgatgtcctgcacttcgagccaagcctcgaacacctgctggtgtgcacgactcacccggttgttgacaccgcgcgcggccgtgcgggctcggtggggcggctctgtcgcccttgccagcgtgagtagcgcgtacctcacctcgcccaacaggtcgcacacagccgattcgtacgccataaagccaggtgagcccaccagctccgtaagttcgggcgctgtgtggctcgtacccgcgcattcaggcggcagggggtctaacgggtctaaggcggcgtgtacgcggccacagcggctctcagcggcccggaaacgtcctcgaaacgacgcatgtgttcctcctggttggtacaggtggttgggggtgctcggctgtcgcggttgttccaccaccagggctcgacgggagagcgggggagtgtgcagttgtggggtggcccctcagcgaaatatctgacttggagctcgtgtcggaccatacaccggtgattaatcgtggtctactaccaagcgtgagccacgtcgccgacgaatttgagcagctctggctgccgtactggccgctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcggccgtcgacggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcgtcccgccctcatgcggatctacctgccgacccggaacgtggacggactcggccgcgcgatctatgccgagtgccacgcgcgaaacgccgaattcccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccagtcggccatctcgcggaagggcgcagcagcgcgcacggcggcgagcacagttgcgcggcgcgcaaagtccgcgtcagccatggaggcattgctatgagcgacggctacagcgacggctacagcgacggctacaaccggcagccgactgtccgcaaaaagccgtgacgcgccgaaggcgctcgaatcaccggactatccgaacgccacgtcgtccggctcgtggcgcaggaacgcagcgagtggctcgccgagcaggctgcacgcgcgcgaagcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggacgggtgtcgcgggggttccgtggggggttccgttgcaacgggtcggacaggtaaaagtcctggtagacgctagttttctggtttgggccatgcctgtctcgttgcgtgtttcgttgcgccgttttgaataccagccagacgagacggggttctacgaatcttggtcgataccaagccatttccgctgaatatcggggagctcaccgccagaatcggtggttgtggtgatgtacgtggcgaactccgttgtagtgcctgtggtggcatccgtggccactctcgttgcacggttcgttgtgccgttacaggccccgttgacagctcaccgaacgtagttaaaacatgctggtcaaactaggtttaccaacgatacgagtcagctcatctagggccagttctaggcgttgttcgttgcgcggttcgttgcgcatgtttcgtgtggttgctagatggctccgcaaccacacgcttcgaggttgagtgcttccagcacgggcgcgatccagaagaacttcgtcgtgcgactgtcctcgttgggatctagcccgcctaatgagcgggctttttttt

SEQ ID NO: 31 - DNA, 8534 bp, Artificial Sequence
  Description of Artificial Sequence Synthetic polynucleotide
cttctagaattccggaattgcactcgccttaggggagtgctaaaaatgatcctggcactcgcgatcagcgagtgccaggtcgggacggtgagacccagccagcaagctgtggtcgtccgtcgcgggcactgcacccggccagcgtaagtaatgggggttgtcggcacccggtgacctagacacatgcatgcatgcttaattaattaagcgatatccggaggaatcacttccatatgcacgctgtgactcgtccgaccctgcgtgaggctgtcgcccgcctagccccgggcactgggctgcgggacggcctggagcgtatcctgcgcggccgcactggtgccctgatcgtgctgggccatgacgagaatgtcgaggccatctgcgatggtggcttctccctcgatgtccgctatgcagcaacccggctacgcgagctgtgcaagatggacggcgccgtggtgctgtccaccgacggcagccgcatcgtgcgggccaacgtgcaactggtaccggatccgtcgatccccaccgacgaatcggggacccggcaccgctcggccgagcgggccgcgatccagaccggttacccggtgatctcagtgagccactcgatgaacatcgtgaccgtctacgtccgcggggaacgtcacgtattgaccgactcggcaaccatcctgtcgcgggccaaccaggccatcgcaaccctggagcggtacaaaaccaggctcgacgaggtcagccggcaactgtccagggcagaaatcgaggacttcgtcacgctgcgcgatgtgatgacggtggtgcaacgcctcgagctggtccggcgaatcgggctggtgatcgactacgacgtggtcgaactcggcactgatggtcgtcagctgcggctgcagctcgacgagttgctcggcggcaacgacaccgcccgggaattgatcgtgcgcgattaccacgccaacccggaaccaccgtccacggggcaaatcaatgccaccctggacgaactggacgccctgtcggacggcgacctcctcgatttcaccgcgctggcaaaggttttcggatatccgacgaccacggaagcgcaggattcgacgctgagcccgcgtggctaccgcgcgatggccggtatcccccggctccagttcgcccatgccgacctgctggtccgggcgttcggaacgttgcagggtctgctggcggccagcgccggcgatctgcaatcagtggacggcatcggcgccatgtgggcccgtcatgtgcgcgaggggttgtcacagctggcggaatcgaccatcagcgatcaataacgcgttctggcgtaatagcgaagaggcccgcaccgatcgcccttcccaacagttgcgcagcctgaatggcgaatggcgctttgcctggtttccggtcgaagcttggccggatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcggggatccgctcggaggcgcggtcgcggcgcggctgtggcatgtcggggcgtgccgctcccccggcgccgcccatcggcccgcccattggcattccgcccatgccgcccatcattcctgtggagccagaactgatccagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgatgatcagctatcccacaccgagccgggggtctagcagctcgcccgcgttttcgggcacaaatgccggatcgtggcccatgtcgatcggtttgttgtaagcgtcgacaaacacgatccgcggctggtatgtgcgggcccgggcgtcgtccatcgtcgcgtacgcaatcagaatcaccagatcccccggatgcaccaagtgcgcggcggcaccgttgatgccaatcacaccactgccgcgttcgccggtgatcgcgtaggtgaccagtcgagcaccgttgtcgatatcgacgatggttacctgttcgccttccagcaggtcggcggcgtccatcaagtcggcatcgatggtcaccgagccgacgtagtgcaggtcggcgcaggtcaccgtggcgcggtggatcttcgacttcagcatcgtccgtaacatcagtttctccaatgtgattcgaggattgcccggtatccgtccgggcggtcggtgccggcgaaagttccgatttcaatcgcaatgttgtccagcagcctggtggtgccaagccgggcagcaaccagcagccgaccggaaccgttgagcggcatcgggccaagcccgatatcgcgcagctccaggtagtcgaccgccacgccgggtgcagcgtcgagcaccgcacgggcggcatccagcgcggcctgcgcgccagccgttgccgcatgcgctgcggccgttagcgccgccgagagcgcgacggccgccgcacgctgggccgggtccaggtagcggttgcgcgacgacatcgccagcccgtcggcttcgcgcacggtcggcacgccgaccaccgcgacatcgaggttgaagtccgcgaccagctgccggatcagcaccagctgctggtagtccttctcaccgaagaacacccgatccgggcgcacgatctgcagcagctttagcacgaccgtcagcacgccggcgaaatgggttggccgcgggccgccctcgagttcggcggccaacggaccgggttgcacggtggtgcgcaggccgtcgggatacatcgccgcggtagttggcgtgaaagcgatttccacgccttcggcccgcagttgcgccaggtcgtcgtccggggtgcggggataggcgtcgagatcttccccggcaccgaattgcatcgggttgacgaagatcgacacgacgacgaccgatccgggcacccgcttggccgcacgcaccaacgcgaggtggccttcgtgcagcgcacccatagtaggcaccaacatcactcgccggccggtgagtcgcagtgcgcgactgacatcggcgacatcccccggtgccgagtacacattgagttcaccgggatggaacgcaggaatcgtcatgccgtcaaaacctcgacgacatccgcgggggcgtgtgcgcgctgcgcggtccgcagcgcgtttatccggtatgcctgggccagcgctgcgtcgacgtccgcgagggccgccagatgatccgcgaccgctgccgcatcgccgcgggcgaccggtccggtgagcgcggcctgtccccgctgcagcgtgttctccagcgccgctctggccagcggcccgacgatgcgctccacgatcccgcccggctggtcgtactagtaacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccagggaattcccatcgagccgagaacgttatcgaagttggtcatgtgtaatcccctcgtttgaactttggattaagcgtagatacacccttggacaagccagttggattcggagacaagcaaattcagccttaaaaagggcgaggccctgcggtggtggaacaccgcagggcctctaaccgctcgacgcgctgcaccaaccagcccgcgaacggctggcagccagcgtaaggcgcggctcatcgggcggcgttcgccacgatgtcctgcacttcgagccaagcctcgaacacctgctggtgtgcacgactcacccggttgttgacaccgcgcgcggccgtgcgggctcggtggggcggctctgtcgcccttgccagcgtgagtagcgcgtacctcacctcgcccaacaggtcgcacacagccgattcgtacgccataaagccaggtgagcccaccagctccgtaagttcgggcgctgtgtggctcgtacccgcgcattcaggcggcagggggtctaacgggtctaaggcggcgtgtacgcggccacagcggctctcagcggcccggaaacgtcctcgaaacgacgcatgtgttcctcctggttggtacaggtggttgggggtgctcggctgtcgcggttgttccaccaccagggctcgacgggagagcgggggagtgtgcagttgtggggtggcccctcagcgaaatatctgacttggagctcgtgtcggaccatacaccggtgattaatcgtggtctactaccaagcgtgagccacgtcgccgacgaatttgagcagctctggctgccgtactggccgctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcggccgtcgacggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcgtcccgccctcatgcggatctacctgccgacccggaacgtggacggactcggccgcgcgatctatgccgagtgccacgcgcgaaacgccgaattcccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccagtcggccatctcgcggaagggcgcagcagcgcgcacggcggcgagcacagttgcgcggcgcgcaaagtccgcgtcagccatggaggcattgctatgagcgacggctacagcgacggctacagcgacggctacaaccggcagccgactgtccgcaaaaagccgtgacgcgccgaaggcgctcgaatcaccggactatccgaacgccacgtcgtccggctcgtggcgcaggaacgcagcgagtggctcgccgagcaggctgcacgcgcgcgaagcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggacgggtgtcgcgggggttccgtggggggttccgttgcaacgggtcggacaggtaaaagtcctggtagacgctagttttctggtttgggccatgcctgtctcgttgcgtgtttcgttgcgccgttttgaataccagccagacgagacggggttctacgaatcttggtcgataccaagccatttccgctgaatatcggggagctcaccgccagaatcggtggttgtggtgatgtacgtggcgaactccgttgtagtgcctgtggtggcatccgtggccactctcgttgcacggttcgttgtgccgttacaggccccgttgacagctcaccgaacgtagttaaaacatgctggtcaaactaggtttaccaacgatacgagtcagctcatctagggccagttctaggcgttgttcgttgcgcggttcgttgcgcatgtttcgtgtggttgctagatggctccgcaaccacacgcttcgaggttgagtgcttccagcacgggcgcgatccagaagaacttcgtcgtgcgactgtcctcgttgggatctagcccgcctaatgagcgggcttttttttggatc

SEQ ID NO: 32 - DNA, 8801 bp, Artificial Sequence
  Description of Artificial Sequence Synthetic polynucleotide
  modified_base (5756)..(5756): a, c, t, g, unknown or other
cggaccgctaccggacagcgaggtccgcgccatcgccaacagcatttggcgttggatcacaaccaagtcgcgcatttgggcggacgggatcgtggtctacgaggccacactcagtgcgcgccagtcggccatctcgcggaagggcgcagcagcgcgcacggcggcgagcacagttgcgcggcgcgcaaagtccgcgtcagccatggaggcattgctatgagcgacggctacagcgacggctacaaccggcagccgactgtccgcaaaaagcggcgcgtgaccgccgccgaaggcgctcgaatcaccggactatccgaacgccacgtcgtccggctcgtggcgcaggaacgcagcgagtggctcgccgagcaggctgcacgccgcgaacgcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggagcgggtgtcgcgggggttccgtggggggttccgttgcaacgggtcggacaggtaaaagtcctggtagacgctagttttctggtttgggccatgcctgtctcgttgcgtgtttcgttgcgcccgttttgaataccagccagacgagacggggttctacgaatcttggtcgataccaagccatttccgctgaatatcggggagctcaccgccagaatcggtggttgtggtgatgtacgtggcgaactccgttgtagtgcctgtggtggcatccgtggcgcggccgcggtaccagatctttaaatctagataaagaagtgacgcggtctcaagcgtcgagcgtcgccagcgtgtcgaggatgtcgaagtcgtagccgtcggcgctggcgatgtagacctgctggtcgaattgactgtcgcgcatacacatcgggccccggggcccgtcgaacccgacatcgtgcgcggatgccatcaggtccggtatctcgggggagtgggcccgctggaagatggcctcgagcgcaagcagaccctcgtaacaggattcggccatcgcgttgagcggtggcgcgtcggcgccgtagcgggcgacgtagctgcccatcaggtccatggcacccgcggtggccagtgaactgaagtacgccgcggcgacatagaggttttcggtggagccggcgccgctggccagcagcatgttctcctccatcagcgggctgaaccgcgccatgcggtcgtgcccgccggcgcgcgcgaactcgcggttgaacaacacggcgtcctggccgacgagcagcatcaacacggcctgcgcccccgacgcgatggccttgcggacaggtgcgcggaaatcgtcggtgccgtacgggacgtagatctcccgtctgagctcgaggtccagatctcggcagtacgcgcgggcggccgcggcggaacggcgcggccagatgtagtcatcgccgaccaggcaccaggaccggatgccgaagtggtcgcgcagccaggcgagcgcgggcgcgatctggatctgcggtgtctcgcctgtgcagaacacgcccggtgtgcgttcaccgccctcgtacaacgaggtgtagacgtacgggatgcggtcgcggaccaccggggagatgcggttgcgcacggccgagatgtgccagccggtcacggcgtcgagaccgtgacctcgcaaccggtcggcgacggtccgggcgacgtcgtcgccgggcgctccgccgtcgagcacctcgatggtgaccttgcggccctgcaggccgcctcggtcgttgacctccttggccgcgagctcggccacggcctcgcacgaaggcgcgaagattcccgctggcccttgaagcggaatcaccagcccgacgcggaactcaacctcgccgtcctgcactccagatcaccgtcgatcccgtgtagtctgcgcttcaaagctttctagcagaaataattcattctgaacagaccccgccgtcgacacgaggagacacccaccatggccgccggacagcagcgccgccccaacctcctgctgccgttggtgcgtctgacccacctcgcggagtcggcgatcgaacgcgtgctcgcggactcgtcgctcaagatcgaggactggcgcgtgctcgacgagttggccggacggcgcaccgtgcccatgagcgatctcgcgcaggccacgctgatcacgggtccgactctcaccagaaccgtcgatcgccttgtgtcgcaagggatcatctaccggactgccgatctgcatgaccgccggcgggtgctcgtggcgttgaccccgcgggggcggacgctgcgcaaccgcctggtggacgcggtagccgaggccgagtgtgcggcttttgaatcgtgcgggctggacgtcgaccagttgcgcgaactcgtcgacaccacctcgaatttgacttcgtaaccacccgcgcccggcgcgggcgttcacccttgacttttattttcatctggatatatttcgggtgaatggaaaggggtgaccatgccgacctacacattccgttgttcccactgcggtcccttcgatctcacctgcgcgatctccgagcgcgatgcggcggcgacctgtccggagtgccggacgccggcgcgccgggtcttcggttcggtagggctgacgacattcaccgcgggacatcaccgcgcattcgacgcggcgtccgcgagcgccgaaagtcccacggtggtgaagtcgattcccgcaggcgcggaccgcccgcgggccccgcgccgcaatcccggtctaccgagtctgccgaggtactagcgacatgggtggcgtcgggctcttctacgtgggtgcggtgctcatcatcgacgggctgatgctgctgggccgcatcagcccacgaggcgcaacaccgctgaacttcttcgtcggcggactgcaggtggtgacgcctacggtgctgatcctgcagtccggcggagacgcggccgtgatcttcgcggcctccgggctctacctgttcggcttcacctacctgtgggtggccatcaacaacgtgaccgactgggacggagaaggtctcggatggttctcgctgttcgtcgcgatcgccgcactcggctactcgtggcacgcgttcaccgccgaggccgacccggcgttcggggtgatctggctgctgtgggcagtgctgtggttcatgctgttcctgctgctcggcctggggcacgacgcactggggcccgccgtcgggttcgtcgcggtggccgaaggcgtgatcaccgccgccgtgccggccttcctgatcgtgtcgggcaactgggaaaccggcccgctccccgccgcggtcatcgccgtgatcggttttgccgcagttgttctcgcataccccatcgggcgccgtctcgcagcgccgtcagtcaccaaccctccaccggccgcgctcgcggccaccacccgataagagaaagggagtccacatatgtaacggatccagctgcagaattcgaagcttatcgatgtcgacgtagttacgagatcggcggccgcatatgagtgtgcccacacaggacggaatgcaccggttcgtcgacgaggacgtctaccacgctgaccggggctcgctgtcggtatccggcgcgaagctgctgttgccgccgtcgtgtcccgcgaaattccgctgggagatggacaacacccggaagccgaaaaaggtctgggacttcggacatgtcgcgcacaaactggtgctcggcaagggtgccgagttcgagatcctcgaccccgaggtgcacgggctgaaggcggacggtacgccgtcggagaagccgaccgcgacgggcatgtggcgcaaggccgaggctgaggctcgcaaacagggcaaggtgccgattcacgtcgacctgttcacgaaggcgtacgacatggccgaaaaggtgcgtcagcacccgacagccggcccgatcttcgccaatcctgacggcgaggccgaggtcgcgctgtactacaccgaccccgagaccggcgtgcggctgcgtggccggatcgactggctcactgacgatatcgatgattacaagacgtcgatgaccgcgaacccggccgagctgaaaaccaagttctacaagctcggctatttcatgcaggcggcctggtacatcgatctactggtcgccctcgggctcgccgagaacccgcgattccggttcatcacgcaggagaaagaaccgccctacgtcgtgactccgatccagtacgacgacgaggcgatcgaagaggggcggcgccgcaaccgccaggcgatccggctctacgccgactgcatggaatcgggcaagtggcctgactacagcgacgacgtggtcacgatcagcctgccctcgtgggggctgccgcgaccgcagaccgtcggcgacgtcgtcaccgacagctatatctacgacaccgacccgctcgaagaggccgacccgattgaaggggattacatctatggctgaaaatgctgtcaccaagcaggattcgcccaaggcacccgagacgatctcgcaagtgctgcaggtgctcgtgccgcagctggcgcgtgcagtgcccaagggtatggaccccgaccgcatcgcgcggatcgtgcaaaccgagatccgcaagtcgcgcaacgcgaaagctgctggcatcgctaagcagtccctcgacgactgcacgcaagagtcatttgccggtgcgctgctgacctcggccgcgctcggtctcgagcccggtgtcaacggcgagtgctacctcgtgccctaccgcgacacccggcgcggcgtggtcgagtgccagctgatcatcggctaccagggcatcgtcaaactgttctggcagcacccgcgcgcctcccggatcgacgcgcagtgggtcggcgcgaacgacgaattccattacaccatgggcctcaatccgacgctgaaacacgtgaaggccaagggtgatcggggtaatccggtctacttctacgcgatcgtcgaggtgaccggcgctgagccgctgtgggacgtgttcaccgccgacgagatcagggaattgcgtcgcggcaaggtcggatcctcgggcgacatcaaggacccgcagcgctggatggagcggaagaccgcgctcaaacaggtgctgaagctggcaccgaagacgacgcggctcgacgcggcgatccgcgccgacgatcgcccgggcaccgacctgtcacagtcgcaggcgctcgcgctgccgtcgaccgtcaagccgacggccgactacatcgacggcgagatcgccgagccgcacgaggtcgatacgccaccgaagtcgtcgcgcgcacaacgcgcgcagcgcgccaccgcgccggcgcccgacgtgcagatggccaatcccgatcagctgaagcgcctcggcgagatccagaaggccgaaaagtacaacgacgccgattggttcaagttcctcgccgattcggccggcgtcaaagccacgcgcgccgccgacctcacgttcgacgaggcgaaggctgtcatcgacatgttcgacgggcccaacgcatgagcgccccggcgaattccgacgcggtggttgatctgcaactagcgtacgatcgactgccaggcatcaaataaaacgaaaggctcagtcgaaagactgggcctttcgttttatgccatcatggccgcggtgatcagctagccacctgacgtcnggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgcttgtatgggaagccccatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataatcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcaccaaccgtggctccctcactttctggctggatgatggggcgattcaggcctggtatgagtcagcaacaccttcttcacgaggcagacctcactagttccatgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaggggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcaacgcgtgagcccaccagctccgtaagttcgggcgctgtgtggctcgtacccgcgcattcaggcggcagggggtctaacgggtctaaggcggcgtgtacggccgccacagcggctctcagcggcccggaaacgtcctcgaaacgacgcatgtgttcctcctggttggtacaggtggttgggggtgctcggctgtcgctggtgttccaccaccagggctcgacgggagagcgggggagtgtgcagttgtggggtggcccctcagcgaaatatctgacttggagctcgtgtcggaccatacaccggtgattaatcgtggtctactaccaagcgtgagccacgtcgccgacgaatttgagcagctctggctgccgtactggccgctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcgcgccgtcgatggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcgtcccgccctcatgcggatctacctgccgacccggaacgtggacggactcggccgcgcgatctatgccgagtgccacgcgcgaaacgccgaatttccgtgcaacgacgtgtgtcc

SEQ ID NO: 33 - DNA, 2145 bp, Artificial Sequence
  Description of Artificial Sequence Synthetic polynucleotide
ggggaattcgaggacatcagccggctgccggatacctgcttcgggatcaccgcggccgacgacgtcgggtatgcgatcggacagtcactggttcttgagatgggcggggagccgttttgtgtgcgcgaagacgcccgcatcctctaccacgcggcgctggcccatgcgagcaaccacatcgtcaccgtgctggccgatgcgctcgaggcgttgcgggccgccctgagcgggggggaactgctcggccaacaaaccgtcgacgaccagccgggcgggatcgtggagcgcatcgtcgggccgctggccagagcggcgctggagaacacgctgcagcggggacaggccgcgctcaccggaccggtcgcccgcggcgatgcggcagcggtcgcggatcatctggcggccctcgcggacgtcgacgcagcgctggcccaggcataccggataaacgcgctgcggaccgcgcagcgcgcacacgcccccgcggatgtcgtcgaggttttgacggccttgacataatgtcgcttatcggcttaatcgatctagaccggccgtgcggaattaagccggcccgtaccctgtgaatagaggtccgctgtgacacaagaatccctgttacttctcgaccgtattgattcggatgattcctacgcgagcctgcggaacgaccaggagttctgggagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggttccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggacggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgatctagacccgggacttgacataatgtcgcttatcggcttaccgtgctgctggcgattgacgtccgcaacacccacaccgttgtgggcctgctgtccggaatgaaagagcacgcaaaggtcgtgcagcagtggcggatacgcaccgaatccgaagtcaccgccgacgaactggcactgacgatcgacgggctgatcggcgaggattccgagcggctcaccggtaccgccgccttgtccacggtcccgtccgtgctgcacgaggtgcggataatgctcgaccagtactggccgtcggtgccgcacgtgctgatcgagcccggagtacgcaccgggatccctttgctcgtcgacaacccgaaggaagtgggcgcagaccgcatcgtgaactgtttggccgcctatgaccggttccggaaggccgccatcgtcgttgactttggatcctcgatctgtgttgatgttgtatcggccaagggtgaatttcttggcggcgccatcgcgcccggggtgcaggtgtcttccgatgccgcggcggcccgcaagcttggg

SEQ ID NO: 34 - DNA, 1128 bp, Artificial Sequence
  Description of Artificial Sequence Synthetic polynucleotide
acttgacataatgtcgcttatcggcttaatcgatctagaccggccgtgcggaattaagccggcccgtaccctgtgaatagaggtccgctgtgacacaagaatccctgttacttctcgaccgtattgattcggatgattcctacgcgagcctgcggaacgaccaggagttctgggagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggttccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggacggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgatctagacccgggacttgacataatgtcgcttatcggctta

SEQ ID NO: 35 - DNA, 3879 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggatgcatcagatctctcgagacttgacataatgtcgcttatcggcttaatcgatctagaccggccgtgcggaattaagccggcccgtaccctgtgaatagaggtccgctgtgacacaagaatccctgttacttctcgaccgtattgattcggatgattcctacgcgagcctgcggaacgaccaggagttctgggagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggttccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggacggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgatctagacccgggacttgacataatgtcgcttatcggcttactcgagattatccatggcggccgcactagtctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 36 - DNA, 4891 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggatgcatcagatctctcgaggaattcgaggacatcagccggctgccggatacctgcttcgggatcaccgcggccgacgacgtcgggtatgcgatcggacagtcactggttcttgagatgggcggggagccgttttgtgtgcgcgaagacgcccgcatcctctaccacgcggcgctggcccatgcgagcaaccacatcgtcaccgtgctggccgatgcgctcgaggcgttgcgggccgccctgagcgggggggaactgctcggccaacaaaccgtcgacgaccagccgggcgggatcgtggagcgcatcgtcgggccgctggccagagcggcgctggagaacacgctgcagcggggacaggccgcgctcaccggaccggtcgcccgcggcgatgcggcagcggtcgcggatcatctggcggccctcgcggacgtcgacgcagcgctggcccaggcataccggataaacgcgctgcggaccgcgcagcgcgcacacgcccccgcggatgtcgtcgaggttttgacggcacttgacataatgtcgcttatcggcttaatcgatctagaccggccgtgcggaattaagccggcccgtaccctgtgaatagaggtccgctgtgacacaagaatccctgttacttctcgaccgtattgattcggatgattcctacgcgagcctgcggaacgaccaggagttctgggagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggttccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggacggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgatctagacccgggacttgacataatgtcgcttatcggcttaccgtgctgctggcgattgacgtccgcaacacccacaccgttgtgggcctgctgtccggaatgaaagagcacgcaaaggtcgtgcagcagtggcggatacgcaccgaatccgaagtcaccgccgacgaactggcactgacgatcgacgggctgatcggcgaggattccgagcggctcaccggtaccgccgccttgtccacggtcccgtccgtgctgcacgaggtgcggataatgctcgaccagtactggccgtcggtgccgcacgtgctgatcgagcccggagtacgcaccgggatccctttgctcgtcgacaacccgaaggaagtgggcgcagaccgcatcgtgaactgtttggccgcctatgaccggttccggaaggccgccatcgtcgttgactttggatcctcgatctgtgttgatgttgtatcggccaagggtgaatttcttggcggcgccatcgcgcccggggtgcaggtgtcttccgatgccgcggcggcccgcaagcttctcgagattatccatggcggccgcactagtctgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 37 - DNA, 29 bp, Artificial Sequence
  Description of Artificial Sequence Synthetic primer
ggggactagtaacaccccttgtattactg

SEQ ID NO: 38 - DNA, 31 bp, Artificial Sequence
  Description of Artificial Sequence Synthetic primer
ggggtgatcatcagaattggttaattggttg

SEQ ID NO: 39 - DNA, 21 bp, Artificial Sequence
  Description of Artificial Sequence Synthetic primer
ggggactagtacgaccagccg

SEQ ID NO: 40 - DNA, 24 bp, Artificial Sequence
  Description of Artificial Sequence Synthetic primer
ggggtgatcagctatcccacaccg

SEQ ID NO: 41 - DNA, 935 bp, Mycobacterium bovis
atgacgattcctgcgttccatcccggtgaactcaatgtgtactcggcaccgggggatgtcgccgatgtcagtcgcgcactgcgactcaccggccggcgagtgatgttggtgcctactatgggtgcgctgcacgaaggccacctcgcgttggtgcgtgcggccaagcgggtgcccggatcggtcgtcgtcgtgtcgatcttcgtcaacccgatgcaattcggtgccggggaagatctcgacgcctatccccgcaccccggacgacgacctggcgcaactgcgggccgaaggcgtggaaatcgctttcacgccaactaccgcggcgatgtatcccgacggcctgcgcaccaccgtgcaacccggtccgttggccgccgaactcgagggcggcccgcggccaacccatttcgccggcgtgctgacggtcgtgctaaagctgctgcagatcgtgcgcccggatcgggtgttcttcggtgagaaggactaccagcagctggtgctgatccggcagctggtcgcggacttcaacctcgatgtcgcggtggtcggcgtgccgaccgtgcgcgaagccgacgggctggcgatgtcgtcgcgcaaccgctacctggacccggcccagcgtgcggcggccgtcgcgctctcggcggcgctaacggccgcagcgcatgcggcaacggctggcgcgcaggccgcgctggatgccgcccgtgcggtgctcgacgctgcacccggcgtggcgtggcggtcgactacctggagctgcgcgatatcgggcttggcccgatgccgctcaacggttccggtcggctgctggttgctgcccggcttggcaccaccaggctgctggacaacattgcgattgaaatcggaactttcgccggcaccgaccgcccggacggataccgggcaatcctcgaatcacattggagaaactga

(19) United States
(12) Patent Application Publication
(10) Pub. No.: US 2021/0024940 A1
(43) Pub. Date: Jan. 28, 2021

(54) RECOMBINANT THERAPEUTIC INTERVENTIONS FOR CANCER

(71) Applicant: THE JOHNS HOPKINS UNIVERSITY, Baltimore, MD (US)

(72) Inventors: William R. Bishai, Baltimore, MD (US); Trinity J. Bivalacqua, Baltimore, MD (US); Alok Kumar Singh, Baltimore, MD (US); Monali Praharaj, Baltimore, MD (US); Takahiro Yoshida, Osaka (JP)

(21) Appl. No.: 16/638,943

(22) PCT Filed: Mar. 14, 2019
(86) PCT No.: PCT/US2019/022341

§ 371 Date: Feb. 13, 2020

Related U.S. Application Data
(60) Provisional application No. 62/658,661, filed on Apr. 17, 2018.

(51) Int. Cl. C12N 15/74; A61K 9/00; C07K 16/28; A61K 45/06

(52) U.S. Cl. CPC C12N 15/74; A61K 9/0019; A61K 45/06; C07K 16/2827; C07K 16/2818

(57) ABSTRACT

Described are compositions and methods for treating or preventing cancer in a subject by administering a pharmaceutical composition comprising a strain of Mycobacteria including an expression vector of the present invention into the bladder of a subject. The pharmaceutical composition may be administered by any suitable means including by a catheter.

### STATEMENT OF GOVERNMENTAL INTEREST

This invention was made with government support under grant nos. AI036973, AI037856, awarded by the National Institutes of Health. The government has certain rights in the invention.

### BACKGROUND OF THE INVENTION

Urothelial cancer of the bladder is the most common type of bladder cancer (BC) in North America, South America, Europe and Asia. Non-Muscle Invasive Bladder Cancer (NMIBC) is associated with a high recurrence rate, frequent intravesical treatments, risk of progression to advanced stages and the highest lifetime treatment among all cancers. Intravesical BCG (bacillus Calmette Guerin) instillation has been the standard of care treatment for NMIBC for 30 years. It is effective in 60-70% patients. BCG has shown to be a very effective vehicle for delivery of antigens. Many studies corroborating an underlying immune response skewed towards a Type I interferon and Th1 induced mediated immune response show promise. Efforts to generate recombinant BCG (rBCG) strains for NMIBC have focused on developing strains that augment these anti-tumor immune responses. To date such efforts have not yielded demonstrable improvement over traditional BCG.

### SUMMARY OF THE INVENTION

One embodiment of the present invention is a vector comprising a nucleic acid sequence expressing a protein or functional part thereof that makes a STING agonist including c-di-AMP (also known as 3′-5′ c-di-AMP); c-di-GMP (also known as 3′-5′ c-di-GMP); 3′-3′cGAMP (also known as 3′-5′,3′-5′cGAMP, the product of the Vibrio cholerae DncV protein); 2′-3′cGAMP (also known as 2′-5′,3′-5′ cGAMP, the product of the human cGAS protein) and a combination thereof, as examples. Some vectors of the present invention comprise the nucleic acid sequence selected from the group consisting of a first nucleic acid sequence encoding a Rv1354c protein, or a functional part thereof; a second nucleic acid sequence encoding a 3′-3′cyclic GMP-AMP synthase (DncV) protein, or a functional part thereof; a third nucleic acid sequence encoding a 2′-3′cyclic GMP-AMP synthase (cGAS) protein, or a functional part thereof; a fourth nucleic acid sequence encoding a DNA integrity scanning (disA) protein, or a functional part thereof and a combination thereof. Each of these nucleic acid sequences express proteins that make one or more of the STING agonist as described in the definition section of the specification. Some vectors of the present invention include in addition to one or more of the sequences listed above a fifth nucleic acid sequence encoding a PanC protein and a PanD protein or functional part thereof. Vectors comprising a nucleic acid sequence encoding a PanC protein and a PanD protein or functional part thereof are typically free of an antibiotic resistance genes. Suitable vectors used in the present invention may include vectors that replicate episomally in multiple copies, or vectors that integrate into a bacterial chromosome in single copy or are otherwise present in the bacterial cell. A vector of the present invention may stably integrate into a bacterial genome or it may stably replicate as an episomal plasmid. Suitable third nucleic acid sequences include those that overexpress the cyclase domains of the cyclic GMP-AMP synthase (cGAS) protein. Other suitable third nucleic acid sequence may expresses a cyclic GMP-AMP synthase (cGAS) protein having a regulatory DNA recognition capability that is non-functional. Vectors of the present invention may also include nucleic acid sequences that encode sequences or proteins that knock out the expression of PDE genes of a strain of Mycobacteria used in the present invention.

Another embodiment of the present invention is a strain of Mycobacteria comprising any one of the vectors of the present invention including a vector comprising a protein or functional part thereof that makes a STING agonist. As mentioned above, examples of STING agonist include c-di-AMP (also known as 3′-5′ c-di-AMP); c-di-GMP (also known as 3′-5′ c-di-GMP); 3′-3′cGAMP (also known as 3′-5′,3′-5′cGAMP, the product of the Vibrio cholerae DncV protein); 2′-3′cGAMP (also known as 2′-5′,3′-5′ cGAMP, the product of the human cGAS protein) and a combination thereof, as examples. Examples of suitable nucleic acid sequence includes a nucleic acid sequence selected from the group consisting of a first nucleic acid sequence encoding a Rv1354c protein, or a functional part thereof; a second nucleic acid sequence encoding a 3′-3′cyclic GMP-AMP synthase (DncV) protein, or a functional part thereof, a third nucleic acid sequence encoding a 2′-3′cyclic GMP-AMP synthase (cGAS) protein, or a functional part thereof; a fourth nucleic acid sequence encoding a DNA integrity scanning (disA) protein, or a functional part thereof and a combination thereof. Examples of suitable strains of Mycobacterium used in the present invention includes Mycobacterium tuberculosis, Mycobacterium bovis, or a combination thereof, for example. Another strain used in the present invention is Mycobacterium bacillus Calmette Guerin (BCG). A strain of Mycobacteria used in the present invention maybe a panthothenate auxotroph of BCG lacking its panCD genetic operon. panCD auxotoph strains lack genomic sequences able to encode functional PanC and/or PanD protein. In some embodiments, strains of Mycobacteria that are pantothenate auxotrophs comprise vectors of the present invention including a panCD nucleic acid encoding the PanC and PanD proteins or functional parts thereof. Vectors of the present invention that include panCD nucleic acid sequences are preferably free of antibiotic resistant genes or nucleic acid sequences that encode functional proteins providing antibiotic resistance. Mycobacteria that are pantothenate auxotrophs of the present invention are preferably free of a genomic antibiotic resistant gene or unable to encode functional proteins that provide antibiotic resistance.

Another embodiment of the present invention is a pharmaceutical composition, comprising any one of the strains of Mycobacteria of the present invention, and (ii) a pharmaceutically acceptable carrier.

Another embodiment of the present invention is a method of eliciting a Type 1 interferon response, enhancing the expression of pro-inflammatory cytokine, and/or eliciting trained immunity in subject comprising the steps of: administering a pharmaceutical composition comprising anyone of the strains of the present invention into a subject; and eliciting a Type 1 interferon response, enhancing the expression of pro-inflammatory cytokine, and/or eliciting trained immunity in the subject. In one embodiment, the pharmaceutical composition is administered into the bladder of the subject by a catheter.

Another embodiment is a method of using a strain of Mycobacteria of the present invention to treat or prevent cancer in a subject. The method comprises the steps of: administering a pharmaceutical composition comprising a strain of Mycobacteria comprising a vector expressing a protein that makes a STING agonist or a functional part thereof to a subject having cancer; and treating or preventing cancer in the subject. The present invention may be used to treat or prevent cancers including epithelial cancers, breast cancer, non-muscle invasive bladder cancer, as examples. In some embodiments, the cancer is a BCG-unresponsive non-muscle invasive bladder cancer (BCG-unresponsive NMIBC) and the pharmaceutical composition is administered by intravesical instillation. In some examples, the cancer is a BCG-naïve non-muscle invasive bladder cander (BCG-naïve NMIBC) and the pharmaceutical composition is administered by intravesical instillation. In other examples, the cancer is selected from the group consisting of colon cancer, uterine cancer, cervical cancer, vaginal cancer, esophageal cancer, nasopharyngeal cancer, endobronchial cancer, and a combination thereof and the pharmaceutical composition is administered to a luminal surface of the epithelial cancer. In some embodiments, the cancer is selected from a solid tumor, liquid tumor and the pharmaceutical composition is administered by intratumoral injection and/or by systemic infusion. The methods of the present invention may include the step of administering a checkpoint inhibitor, such as anti-PD1, anti-PDL1, a combination thereof, as example. In another embodiment, the cancer is bladder cancer and a catheter administers the pharmaceutical composition.

One embodiment of the present invention is an expression vector comprising a first nucleic acid sequence encoding a Rv1354c protein, or a functional part thereof; a second nucleic acid sequence encoding a cyclic GMP-AMP synthase (DncV) protein, or a functional part thereof; a third nucleic acid sequence encoding a cyclic GMP-AMP synthase (cGAS) protein, or a functional part thereof, a fourth nucleic acid sequence encoding a DNA integrity scanning (disA) protein which functions as a diadenylate cyclase, or a functional part thereof, or a combination thereof. Some expression vectors of the present invention have a first nucleic acid sequence that overexpresses the cyclase domains of the Rv1354c protein when compared to the expression of a native Rv1354c protein as a reference. Some expression vectors of the present invention have a second nucleic acid sequence that overexpresses the cyclic GMP-AMP synthase (DncV) protein, when compared to the expression of a native DncV protein. Some expression vectors of the present invention have the third nucleic acid sequence that overexpresses the cyclase domains of the cyclic GMP-AMP synthase (cGAS) protein when compared to the expression of a native cGAS protein. Suitable Rv1354 proteins used in the present invention include a Mycobacterium tuberculosis Rv1354 protein. Suitable DncV proteins used in the present invention include a Vibrio cholera DncV protein. Suitable cGAS proteins used in the present invention include a Homo sapiens cGAS protein. Suitable DisA proteins used in the present invention include aMycobacterium tuberculosis disA protein.

Another embodiment of the present invention includes a strain of BCG comprising a cdnP gene, an Rv1354c gene, an Rv1357c gene, or a combination thereof, wherein the cdnP gene is unable to express a functional cyclic di-nucleotide phosphodiesterase (CdnP) protein, the Rv1354c gene is unable to express a functional Rv1345c protein, and/or the Rv1357c gene is unable to express a functional Rv1357 protein. Some BCG strains of the present invention may have an Rv1354c gene that comprises a non-functional EAL domain. The BCG strains of the present invention may comprise any of the expression vectors of the present invention.

Another embodiment of the present invention is a method of treating or preventing bladder cancer comprising the steps of: administering a pharmaceutical composition comprising a strain of BCG including an expression vector of the present invention into the bladder of a subject; and treating or preventing bladder cancer in the subject when compared to a reference subject who was not administered the pharmaceutical composition. The pharmaceutical composition may be administered by any suitable means including by a catheter.

Another embodiment of the present invention is a method of eliciting a Type 1 interferon response in a subject comprising the steps of: administering a pharmaceutical composition comprising a strain of BCG including an expression vector of the present invention into the subject such as the subject's bladder; and enhancing a Type 1 interferon response in the subject compared to a reference subject not administered the pharmaceutical composition.

Another embodiment of the present invention is a method of treating or preventing cancer in a subject comprising the steps of: administering a pharmaceutical composition comprising a strain of BCG including an expression vector of the present invention into a tumor of a subject having cancer; and treating or preventing cancer in the subject when compared to a reference subject not administered the pharmaceutical composition. The pharmaceutical composition may be administered by any suitable means including injection into the tumor. Cancers that may be treated or prevented by this method include, but are not limited to, breast cancer, and/or non-muscle invasive bladder cancer.

Examples of Mycobacteria used in the present invention includes Mycobacterium tuberculosis, Mycobacterium bovis, Mycobacterium bovis Bacillus Calmette Guerin (referred to a BCG), Mycobacterium smegmatis, Mycobacterium avium complex, and other non-tuberculous mycobacteria (NTM). Examples of BCG strains used in the present invention including those that overexpress STING agonists, include BCG Pasteur, BCG-Pasteur-Aeras, BCG Tice (also known as BCG Chicago), BCG-Connaught (also known as BCG Toronto), BCG Danish, BCG-Prague (also known as BCG Czechoslovakian), BCG Russia (also known as BCG Moscow), BCG Moreau (also known as BCG Brazil), BCG Japan (also known as BCG Tokyo), BCG Sweden (also known as BCG Gothenburg), BCG Birkhaug, BCG Glaxo, BCG Frappier (also known as BCG Montreal), BCG Phipps, or other available BCG strains.

Another embodiment of the present invention is a method of treating diabetes comprising the steps of: administering a pharmaceutical composition comprising a strain of Mycobacteria comprising a vector expressing a protein or a functional part thereof that makes a STING agonist to a subject having diabetes; and treating or preventing diabetes in the subject by providing trained immunity. Trained immunity refers to the ability of one antigenic stimulus to elicit more potent immune responses to a second, different antigenic stimulus introduced at a later time. Trained immunity is antigen independent, based on heterologous CD4 and CD8 memory activation, cytokine mediated, and is associated with epigenetic and metabolic changes. The method results in the up-regulation of glycolysis that mediated by the trained immunity. The aforementioned up-regulation of glycolysis is beneficial in preventing and treating Type 1 and Type 2 diabetes mellitus.

Another embodiment of the present invention is a method of stimulating trained immunity in a subject comprising the steps of: administering a pharmaceutical composition comprising a strain of Mycobacteria comprising a vector expressing a protein or a functional part thereof that makes a STING agonist to a subject; and stimulating trained immunity in the subject. Wherein the method up-regulates glycolysis in the subject and/or stimulates episomal changes in histone methylation in the subject that mediates trained immunity in the subject.

Another embodiment of the present invention is a method of treating or preventing a viral infection in a subject comprising the steps of: administering a pharmaceutical composition comprising a strain of Mycobacteria comprising a vector expressing a protein or a functional part thereof that makes a STING agonist to a subject; and treating or preventing the viral infection in the subject. The method stimulates trained immunity in the subject that treats or prevents the viral infection in the subject. Wherein the method up regulates glycolysis in the subject and/or stimulates episomal changes in histone methylation in the subject that mediates trained immunity in the subject.

Another embodiment of the present invention is a method of treating or preventing a bacterial infection, or a drug-resistant bacterial infection in a subject comprising the steps of:

administering a pharmaceutical composition comprising a strain of Mycobacteria comprising a vector expressing a protein or a functional part thereof that makes a STING agonist to a subject; and treating or preventing the bacterial infection or the drug-resistant bacterial infection in the subject. The method stimulates trained immunity in the subject that treats or prevents the bacterial infection in the subject. Wherein the method up regulates glycolysis in the subject and/or stimulates episomal changes in histone methylation in the subject that mediates trained immunity in the subject. The methods of the present invention may use one or more of the vectors of the present invention or one or more strain of bacteria comprising a vector of the present invention.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could, for example, increase the analog's protease resistance, membrane permeability, or half-life, without altering, for example, ligand binding. An analog may include an unnatural amino acid, in another example.

By “cdnP” is meant either 1) a cdnP gene or nucleic acid sequence that encodes a cyclic di-nucleotide phosphodiesterase (cdnP) protein or 2) the cyclic di-nucleotide phosphodiesterase protein. Examples include the M tuberculosis cdnP gene in H37Rv, Rv2837c, having NCBI Gene ID 888920, and a cdnP protein of UniProtKB/Swiss-Prot P71615.2.

By “cGas” is meant either 1) a cGas gene or nucleic acid sequence that encodes a cyclic GMP-AMP synthase (cGAS) protein, or 2) the cyclic GMP-AMP synthase protein. Examples of cGas include the H sapiens cGAS gene (NCBI Gene ID: 115004) and the protein encoded by this gene (UniProtKB/Swiss-Prot: Q8N884.2). The cGas protein is a cyclic GMP-AMP synthase from humans that makes 2′3′cGMP. 2′3′cGMP is a STING agonist in humans.

By “cyclase domains” is meant, of cGAS, for example, is part of the 522 amino acid human cGAS protein described in Kranzusch et al. (Cell Reports 2013; 3:1362-1368 PMID 23707061). A cyclase domain may be described as having an NTase core situated from amino acid 160-330, and a regulatory-sensor domain that is the C-domain situated from amino acids 330-522. Mutants of the NTase core sequence as well as mutants of the regulatory-sensor domain can be used to generate constitutively active variants of cGAMP designed to produce high levels of cGAMP without the normal requirement for activation by DNA binding. Another example of a cyclase domain includes the 623 amino acid M tuberculosis Rv1354c of NCBI Gene ID: 887485, and the protein encoded by this gene (UniProtKB/Swiss-Prot: P9WM13) that encodes a protein capable of both c-di-GMP (cyclic diguanylate or cyclic di-GMP) synthesis (via its GGDEF domain, amino acids 201-400) and degradation (via its EAL domain, amino acids 401-623). The GAF domain (amino acids 1-200) is a regulatory domain. The GGDEF domain as well as mutants of the regulatory-sensor GAF domain and polypeptides truncated to remove the EAL domain (phosphodiesterase activity) can be used to generate constitutively active variants of Rv1354c designed to produce high levels of c-di-GMP.

By “DisA” or “disA” is meant either 1) a Dis A gene or nucleic acid sequence that encodes a DNA integrity scanning (DisA) protein or 2) the DNA integrity scanning protein. Examples include the 358 amino acid M. tuberculosis disA gene Rv3586 of NCBI Gene ID: 887485, and the protein encoded by this gene is UniProtKB/Swiss-Prot: P9WNW5.1. The protein is a diadenylate cyclase as described by Dey & Bishai et al. Nature Medicine 2015;21:401-6. PMID: 25730264. A DisA protein is a diadenylate cyclase that makes c-di-AMP. c-di-AMP is a STING agonist.

By “disease” is meant any condition or disorder that damages or interferes with the normal function of a cell, tissue, or organ. Examples of diseases include, but are not limited to, bladder cancer.

By “dncV” is meant a gene that encodes a Cyclic GMP-AMP synthase that catalyzes the synthesis of 3′3′-cyclic GMP-AMP (3′3′-cGAMP) from GTP and ATP, a second messenger in cell signal transduction. Is also able to produce c-di-AMP and c-di-GMP from ATP and GTP, respectively; however, 3′3′-cGAMP is the dominant molecule produced by DncV in vivo, contrary to the 2′3′-cGAMP produced by eukaryotes. Is required for efficient V. cholerae intestinal colonization, and down-regulates the colonization-influencing process of chemotaxis. Is not active with dATP, TTP, UTP, and CTP. The DncV protein is a cyclic GMP-AMP synthase from V. cholerae that makes 3′3′cGAMP. 3′3′cGAMP is a STING agonist.

By “EAL domain” means a conserved protein domain that is found in diverse bacterial signaling proteins. The EAL domain may function as a diguanylate phosphodiesterase and has been shown to stimulate degradation of a second messenger, cyclic di-GMP. A non-functional EAL domain will not have one or more of these functions. An example of an EAL domain includes the 307 amino acid M tuberculosis Rv1357c gene of NCBI Gene ID: 886815, and the protein encoded by this gene is UniProtKB/Swiss-Prot: P9WM07 that encodes a c-di-GMP phosphodiesterase (PDE) and is comprised of a sole EAL domain. This enzyme's activity is to serve as a c-di-GMP phosphodiesterase, cleaving the cyclic dinucleotide (which has signaling activity) into 2 GMP molecules (which lack signaling activity), as described in the article titled, “A full-length bifunctional protein involved in c-di-GMP turnover is required for long-term survival under nutrient starvation in Mycobacterium smegmatis,” Bharati B K, Sharma I M, Kasetty S, Kumar M, Mukherjee R, Chatterji D. Microbiology. 2012 June; 158(Pt 6):1415-27. doi: 10.1099/mic.0.053892-0. Epub 2012 Feb. 16.PMID: 22343354. Another example of an EAL domain includes the 336 amino acid M. tuberculosis cdnP gene in H37Rv (Rv2837c), a c-di-AMP phosphodiesterase comprising an EAL domain with the capability of hydrolyzing human 2′-3′cGAMP (the product of the human cGAS enzyme) as shown by Jain-Dey Bishai et al. Nat Chem Biol. 2017;13:210-217 PMID 28106876.The structural characteristics of the EAL domains (cyclic dinucleotide phosphodiesterase activity) and GGDEF domains (cyclic dinucleotide cyclization-biosynthetic activity) are known and well described (for example, in Schirmer T, Jenal U. Structural and mechanistic determinants of c-di-GMP signaling. Nat Rev Microbiol. 2009; 7:724-35. PMID: 19756011).

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “dncV” is meant either 1) a dncV gene or nucleic acid sequence that encodes a cyclic GMP-AMP synthase (DncV) protein, or 2) the Cyclic GMP-AMP synthase protein. Examples include, but are not limited to, the Vibrio cholerae dncV gene of NCBI Gene ID: 2614190 and the protein encoded by this gene is UniProtKB/Swiss-Prot: Q9KVG7.1

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain, for example, 10, 20, 30, 40, 50, 60, 70, 80, 90, 100, 200, 300, 400, 500, 600, 700, 800, 900, or 1000 nucleotides or amino acids.

By “Gene deletion” is meant using allelic exchange methodologies well-known to one skilled in the art to delete the full gene coding region of the gene of interest from the chromosome of BCG. Gene replacement with selectable markers such as antibiotic resistance cassettes is a form of allelic exchange and may be performed. Technologies are also available to generate unmarked deletions (no selectable marker) in which the gene is entirely deleted and no selectable marker is introduced in its place.

By “Gene domain deletion” is meant using the above allelic exchange methodologies to remove the portion of a gene encoding a particular domain (in the case of the present invention the EAL domain of Rv1354c which encodes the CDN phosphodiesterase domain of a multifunctional polypeptide) leaving the other portions of the polypeptide intact and in frame.

By “H sapiens” is meant Homo sapiens.

By “obtaining” or as in “obtaining an agent” is meant synthesizing, purchasing, or otherwise acquiring the agent.

By “overexpression” is meant, in a general sense, a gene expressing its corresponding protein in a greater quantity than a wild type or reference gene. An example of creating a gene overexpressing a protein in the present invention includes fusing the DNA encoding the gene of interest to a strong promoter in BCG such as Phsp60 or to a strong conditionally active promoter such as PtetOFF. In PtetOFF, gene expression is turned off in the presence of tetracycline, anhydrotetracycline, or doxycycline; however, when the recombinant BCG is administered as an immunotherapy in a human or an animal model, the gene of interest will be turned on. This conditionally active strategy has the advantage of preventing any deleterious effects on viability or growth rate that strong overexpression of cyclic dinucleotide producing enzyme might have on the BCG organisms while the BCG is being grown, and it allows for strong expression (“overexpression”) only when the BCG immunotherapy is given as a therapeutic to a mammalian host.

By “M.tb” is meant Mycobacterium tuberculosis.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” or “decreases” is meant a negative alteration of at least about 10%, 25%, 50%, 75%, or 100%, for example, or any percentage in between.

By “increases” is meant a positive alteration of at least about 10%, 25%, 50%, 75%, or 100%, for example, or any percentage in between.

By “reference” is meant a standard or control condition.

A “reference sequence” is meant a defined sequence used as a basis for sequence comparison. A reference sequence may be a subset of or the entirety of a specified sequence;

for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “reference BCG strain” is meant, for example, a conventional BCG strain that does not contain the expression vectors of the present invention and/or the endogenous genes unable to express a cdnP functional protein, a Rv1354c functional protein, a Rv1357c functional protein, or a combination thereof.

By “Regulatory DNA Recognition Capability” is meant the ability of a protein to detect or bind DNA. For example a cGAS protein is known to bind DNA, such as cytosolic DNA, and triggers the reaction of GTP and ATP to form cyclic GMP-AMP (cGAMP). cGAMP binds to the Stimulator Interferon Genes (STING) which triggers phosphorylation of IRF3 via TBK1.

By “Rv1354c” is meant either 1) a Rv1354c gene or nucleic acid sequence that encodes a Rv1354c protein or 2) the Rv1354c protein (e.g., Gupta, Kumar, and Chatterji;

PLoS ONE (November, 2010); Vol. 5; Issue 11; and Bhariati, Sharma, Kasetty, Kumar, Mukherjee, and Chatterji; Microbiology (2012), 158, 1415-1427). The Rv1354c protein is a diguanylate cyclase that makes c-di-GMP. C-di-GMP is a STING agonist.

By “Rv1357c” is meant either 1) a Rv1357 gene or nucleic acid sequence that encodes a cyclic di-GMP phosphodiesterase protein (Rv1357) protein or 2) the cyclic di-GMP phosphodiesterase protein (e.g., Gupta, Kumar, and Chatterji; PLoS ONE (November, 2010); Vol. 5; Issue 11; and Bhariati, Sharma, Kasetty, Kumar, Mukherjee, and Chatterji; Microbiology(2012), 158, 1415-1427). The Rv1357c protein is a diguanylate cyclase that mkes c-di-GMP. C-di-GMP is a STING agonist.

By “STING agonist” is meant a molecule which binds to STING (stimulator of interferon genes, or TMEM173), activates it, and triggers activation of the IRF3-TBK1 pathway leading to increased transcription of type 1 interferon and other genes.

By “CDN” is meant cyclic dinuculeotide such as 3′-5′ c-di-AMP, 3′-5′ c-di-GMP, 3′-3′ cGAMP (also known as 3′-5′,3′-5′cGAMP, the product of the Vibrio cholerae DncV protein), or 2′-3′ cGAMP (also known as 2′-5′,3′-5′ cGAMP, the product of the human cGAS protein).

By “PAMP” is meant pathogen associated molecular pattern. PAMPs are microbial products including small molecules which are recognized by innate immune sensors. Examples of PAMPs are 3′-5′ c-di-AMP, 3′-5′ c-di-GMP, 3′-3′ cGAMP,

By “DAMP” is meant danger associated molecular pattern. DAMPs are host-derived (that is human, mouse, or other mammalian model of disease) molecules that are produced to signal danger such as infection or other derangement of normal physiology. An example of a DAMP is 2′-3′ cGAMP which is produced by the host sensor enzyme cGAS upon detection of double-stranded DNA in the cytosol as occurs during viral or certain intracellular bacterial infections.

By “panCD” is meant the genetic operon from bacteria or other species the encodes the biosynthetic gene panC (encoding the PanC protein which has pantoate-beta-alanine ligase enzymatic activity) and the biosynthetic gene panD (encoding the PanD protein which has aspartate 1-decarboxylase enzymatic activity). The PanC and PanD proteins are required for the biosynthesis of pantothenic acid or pantothenate also called vitamin B₅ (a B vitamin). Pantothenic acid, a water-soluble vitamin, is an essential nutrient for bacteria and for all mycobacteria including BCG. Pantothenic acid is required in order to synthesize coenzyme-A (CoA), as well as to synthesize and metabolize proteins, carbohydrates, and fats.

By “specifically binds” is meant a compound, nucleic acid, peptide, protein, or antibody, for example, that recognizes and binds a polypeptide or nucleic acid sequence, but which does not substantially recognize and bind other molecules in a sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

By “sensitivity” is meant the percentage of subjects with a particular disease.

By “specificity” is meant the percentage of subjects correctly identified as having a particular disease, i.e., normal or healthy subjects. For example, the specificity is calculated as the number of subjects with a particular disease as compared to non-cancer subjects (e.g., normal healthy subjects).

By “trained immunity” is meant the ability of one antigenic stimulus to elicit more potent immune responses to a second, different antigen administered at a later time. Trained immunity is antigen-independent, based on heterologous CD4 and CD8 memory activation, cytokine mediated, and is associated with epigenetic and metabolic changes.

By “Phsp60” or “Phsp65” is meant a strong mycobacterial promoter derived from the Mycobacterium leprae Hsp65 5′UTR.

By “5′UTR” is meant the 5′ untranslated region of a gene.

By “3′UTR” is meant the 3′ untranslated region of a gene.

By “WT” is meant wild type.

By “BCG-WT” is meant a wild type strain of Mycobacterium bovis bacillus Calmette Guerin.

As used herein, ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for bladder cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like). In particular embodiments, determination of subjects susceptible to or having a pancreatic cancer is determined by measuring levels of at least one of the markers.

### BRIEF DESCRIPTION OF THE DRAWINGS

FIG. 1A-1B Mycobacteria overexpressing disA from the pSD5B Pₕₛₚ₆₀::disA plasmid construct release large amounts of c-di-AMP into the macrophage cytosol and transcribe high levels of disA mRNA. A. J774 macrophages infected with M.tb harboring the pSD5B Pₕₛₚ₆₀::disA plasmid or wild type M.tb (CDC1551) at an MOI of 1:20. Intramacrophage levels of c-di-AMP were determined by LC-MS/MS after 24 hours of infection. As can be seen, the M.tb-disA-OE strain produces ˜15-fold more c-di-AMP than wild type M.tb (CDC1551). The BCG-disA-OE would be expected to show similarly high levels of c-di-AMP. (Data are from Dey B, Dey R J, Cheung L S, Pokkali S, Guo H, Lee J H, Bishai W R. A bacterial cyclic dinucleotide activates the cytosolic surveillance pathway and mediates innate resistance to tuberculosis. Nat Med. 2015; 21:401-6. PMID: 25730264.) B. BCG-Pasteur harboring the pSD5B Pₕₛₚ₆₀::disA plasmid or BCG-Pasteur-WT were grown to mid-exponential phase. Bacteria were lysed and mRNA was prepared. The levels of disA mRNA were determined by quantitative RT-PCR. The BCG-disA-OE strain produces ˜50-fold more disA mRNA than BCG-Pasteur-WT.

FIG. 2. BCG overexpressing disA augments pro-inflammatory cytokines. Gene expression profiling (qPCR) of pro-inflammatory cytokines and IFN-β in mouse BMDMs challenged with wild-type and disA overexpression strains of BCG-Pasteur.

FIG. 3. BCG overexpressing disA augments IRF3 signaling. Effect of disA overexpression on activation of IRF pathway measured by IRF-SEAP QUANTI Blue reporter assay. The culture supernatants of infected RAW-Blue ISG cells were assayed for IRF activation. The image below the IRF-activation graph represents QUANTI Blue assay plate and sample wells; treatment parameters for column of wells correspond to those defined for the bars above aligned with the wells. BCG-disA-OE in this figure is derived from BCG Pasteur.

FIG. 4A-4C. Increased pro-inflammatory cytokines in response to disA overexpression. Differential expression of TNF-α (A), IL-6 (B) and IL-1β (C) in mouse BMDMs challenged with wild-type and disA overexpression strains of BCG-Pasteur. Culture supernatants were assayed by ELISA for different cytokines.

FIG. 5. BCG overexpressing disA induces differential immune response in human bladder cancer cells (RT4). Differential gene expression in human RT4 bladder cancer cells challenged with wild-type BCG-Pasteur, wild-type BCG-Tice strain, and BCG-Pasteur-disA-OE Expression levels of mRNA was measured using a SYBR green-based quantitative real-time PCR.

FIG. 6. Schematic workflow of testing relative therapeutic efficacy of wild-type and BCG-disA-OE strains.

FIG. 7. Tumor involvement index of tumor-bearing rats untreated or treated with WT BCG or rBCG overexpressing disA (rBCG=BCG-Pasteur-disA-OE; wtBCG=BCG-Pasteur).

FIG. 8. Immune profiling of MNU-induced Fisher rat urinary bladder tumors in response to intravesical therapy using different strains of BCG. Differential gene expression in Rat bladder tumor cells after therapy with wild-type and disA overexpression strains of Mycobacterium bovis BCG-Pasteur. Expression levels of mRNA were measured using a TaqMan-based quantitative real-time PCR. BCG-WT is BCG Pasteur and BCG-disA-OE was derived from BCG Pasteur in this figure.

FIG. 9. Gene expression profiling of bladders from MNU tumor bearing rats untreated or treated with WT or rBCG overexpressing disA.

FIG. 10. Summary of relative gene expression by BCG-disA-OE versus BCG-WT in different cells or tissues. Mouse bone marrow-derived macrophages (BMDM), human immortalized bladder cancer cell lines RT4 and 5637, and rat immortalized bladder cancer cell lines were infected with BCG-disA-OE and BCG-WT for 24 hours and mRNA was prepared from the cells. Rats were exposed to MNU by intravesical instillation over 8 weeks and then treated with either BCG-disA-OE or BCG-WT by intravesical instillation for 8 weeks. Bladders were removed upon necropsy at week 16, and mRNA was prepared. Quantitative RT-PCR for the cytokine or chemokine genes indicated was performed. The changes shown are the fold-induction or reduction observed with BCG-disA-OE normalized to that seen with BCG-WT. BCG-WT is BCG Pasteur and BCG-disA-OE was derived from BCG Pasteur in this figure.

FIG. 11. Proposed mechanisms of action of BCG overexpressing disA.

FIG. 12. Molecular genetic modifications to BCG which will increase the levels of CDN PAMP and DAMP molecules which are STING agonists. PAMP: pathogen associated molecular pattern (made by bacteria): c-di-AMP (disA), c-di-GMP (Rv1354c), 3′3′-cGAMP (dncV). DAMP: danger-associated molecular pattern (made by host): 2′-3′-cGAMP. OE: overexpressor. KO: knockout (gene replacement).

FIG. 13. Diagram of two cyclic dinucleotide cyclase and phosphodiesterase proteins present in BCG: BCG_RS07340 and BCG AHM07112. BCG_RS07340 is a bifunctional protein with both CDN cyclase and CDN PDE activities. BCG AHM07112 is a CDN PDE. The domains are: GAF (regulatory), GGDEF (diguanylate cyclase), and EAL diguanylate phosphodiesterase.

FIG. 14. M tuberculosis harboring the pSD5B Phsₚ6o::disA plasmid (M.tb-disA-OE or M.tb-OE) is significantly attenuated for virulence in mice compared to wild type M.tb (M.tb-CDC1551). 6-7-week-old female BALB/c mice (n=10 per group) were infected as described above with ˜3.5 log₁₀ CFU by aerosol infection. Day 1 CFU counts were performed on 3 mice in each group and confirmed the implantation of 3.5 log10 CFU units. Mice were held until death. As can be seen, the median time to death for wild-type M. tuberculosis infection was 150.5 days. In contrast, mice infected with the same inoculum of M.tb-disA-OE (M.tb-OE) had a median time to death of 321.5 days (p<0.001). The BCG-disA-OE is expected to show similar loss of virulence in mice compared with BCG-WT. (Data are from Dey B, Dey R J, Cheung L S, Pokkali S, Guo H, Lee J H, and Bishai W R. A bacterial cyclic dinucleotide activates the cytosolic surveillance pathway and mediates innate resistance to tuberculosis. Nat Med. 2015; 21: 401-6. PMID: 25730264.)

FIG. 15A-15B. Other BCG strains are also active: BCG Tice strain overexpressing disA also shows induction of proinflammatory cytokines similar to BCG Pasteur overexpressing disA. Bone marrow derived macrophages were challenged with wild-type and disA overexpressing strains of both BCG Pasteur and BCG Tice strains at an M.O.I of 1:20 for 15 h. Culture supernatants were harvested and probed for cytokines using ELISA. Differential expression pattern of TNF-α (A) and IL-6 (B) in mouse BMDM challenged with the two different strains of BCG. The BCG-Tice strain was from the commercially available Onco-Tice product.

FIG. 16. The Type I interferon responses in macrophages in response to BCG-disA-OE are STING-dependent. Bone marrow-derived macrophages from STING-ablated (KO) and control mouse were challenged with wild-type and disA OE strains of BCG Pasteur for 24 h. Culture supernatants were probed for IFN-β levels using ELISA.

FIG. 17 Shows that intravesical instillation of BCG-disA-OE displays greatest antitumor efficacy (statistically significant improvement in pathology) in the MNU carcinogen model of non-muscle invasive bladder cancer (NIMBC). Groups of rats received 4 intravesical treatments with MNU over the first 8 weeks (one treatment every 2 weeks) to elicit NIMBC. Over the next 8 weeks they received 4 intravesical treatments with either PBS (untreated), BCG-WT, or BCG-disA-OE (one treatment every 2 weeks). At the end of the 16-week experiment, rats were sacrificed, and their bladders were removed. A portion of the bladder was fixed and subjected to H&E staining and then interpreted in a blinded fashion by a Board-certified urologic pathologist. The tumor involvement score and cancer stage (T2-3, T1, CIS+papillary lesions, CIS alone, or normal-dysplastic) were determined and are shown. As may be seen BCG-disA-OE instillation resulted into statistically significantly and lower tumor involvement index than PBS (untreated) while BCG-WT was not statistically significantly superior to PBS. This 16 -week experiment was performed twice. The data in FIG. 7 represent the results of Experiment 1. The data in this figure (FIG. 17) represent the combined results of Expt 1 plus Expt 2. The qPCR data shown in FIG. 8 and FIG. 9 were obtained using bladder tissue at necropsy from the end of Expt 1.

FIG. 18 shows that BCG-disA-OE reduces Tregs (CD4⁺CD25⁺Foxp3⁺) in murine syngeneic bladder cancer tumors. Mice were implanted on the flank with 5×10⁶ BBN975 murine bladder cancer tumor cells. When the tumors were 1.5 cm in diameter, mice received 3 intratumoral injections of either PBS (control), BCG-WT, or BCG-disA-OE (one treatment every 2 days). Two days after the last intratumoral treatment, mice were sacrificed and their spleens and tumors were removed. After tumor cell dispersal, the cell preparations were stained and subjected to flow cytometry. As may be seen BCG-disA-OE led to reduced tumor CD4⁺ Tregs, reduced tumor CD8⁺ Tregs, and reduced spleen CD4⁺ Tregs.

FIG. 19 shows that BCG-disA-OE is safer than BCG-WT in two mouse models. In Panel A, groups of BALB/c mice (immunocompetent) were exposed to 1×10³ CFU (confirmed by sacrificing a group of mice and determining day 1 lung CFU counts) of either BCG-WT or BCG-disA-OE using a Glas-Col aerosolization chamber. After 4 weeks, the mice were sacrificed from each group, their lungs were removed, homogenized, and plated on 7H11 agar plates. The figure shows the mean CFU counts for the BCG-WT and BCG-disA-OE-infected mouse lungs. As may be seen a statistically significantly lower lung CFU burden was observed with BCG-disA-OE compared with BCG-WT. In Panel B groups of SCID mice (immunosuppressed) were exposed to 1×10² CFU (confirmed by sacrificing a group of mice and determining day 1 lung CFU counts) of either BCG-WT or BCG-disA-OE using a Glas-Col aerosolization chamber. A third group was uninfected. The figure shows a Kaplan-Meier survival curve for the groups of mice. As may be seen BCG-disA-OE-infected mice had a statistically significantly longer survival time than BCG-WT-infected mice.

FIG. 20 shows that BCG-disA-OE elicits statistically significantly higher levels of “Trained Immunity immunological and epigenetic marks” in CD14⁺ human monocytes than does BCG-WT. “Trained Immunity” refers to the ability of a first immunologic stimulus to induce increased immune responses to a second antigenically different stimulus give subsequently. In this experiment, CD14⁺human monocytes were prepared from LeukoPaks collected by apheresis. On day 0 they were infected with either BCG-WT or BCG-disA-OE at a MOI of 5:1 for 3 hours. A third group of cells were not infected. After infection, cells were washed multiple times (every two days). After a 6-day rest period, the monocytes were re-stimulated with the TLR1/2 agonist PAM3CSK4 for 2 hours. Cells were washed repeatedly and were subsequently incubated for 24 h. Th levels of secreted IL-1β were measured in the culture supernatants by ELISA. As may be seen, while BCG-WT itself elicited statistically significantly higher levels of immune response to the second stimulus compared to uninfected cells, BCG-disA-OE elicit statistically significantly more of a response than either BCG-WT or uninfected cells.

FIG. 21 shows that BCG-disA-OE elicits a greater histone activation mark (H3K4-trimethylation) in the IL6 and TNF gene promoter regions than BCG-WT. “Trained Immunity” refers to the ability of a first immunologic stimulus to induce increased immune responses to a second antigenically different stimulus give subsequently. Trained immunity has been associated with epigenetic modifications, such as histone methylation, in the promoter regions of cytokines and other immune mediators. The experiment shown in FIG. 21 was performed in the same set of cells and exactly the same way as that described in FIG. 20 except that after the second stimulus with the TLR1/2 agonist PAM3CSK4 (abbreviated PAM3), cells were harvested fixed, chromatins were cross-linked and DNA was collected for chromatin immunoprecipitation analysis (ChIP) using an antibody specific for the H3K4-me3 histone methylation mark. H3K4-me3 is known to be a gene activating mark. The graph shows the relative fold change in abundance of immunoprecipitated DNA as measured by quantitative PCR using primers for the IL6 and TNF gene promoter region. As may be seen both BCG-Pasteur-disA-OE and BCG-Tice-disA-OE led to significantly greater levels of H3K4 histone trimethylation in the IL6 and TNF promoter regions than did their corresponding BCG-WT strains following challenge with the second stimulus, PAM3CSK4.

FIG. 22 shows the successful construction of BCG-Tice-disA-OE. The inventors' previous work had utilized BCG-Pasteur to construct BCG-Pasteur-disA-OE. This strain was provided to one of the inventors by Dr. Frank Collins in 1995. It is the same strain known as BCG-Pasteur-Aeras. BCG-Tice is manufactured and sold by Merck, and is the sole FDA-approved BCG available in the United States. The inventors purchase BCG-Tice, prepared electrocompetent BCG-Tice, and electroporated the pSD5-hsp60-MT3692 plasmid into BCG-Tice. The drawing shows the results of colony PCRs for 5 kanamycin-resistant candidate clones of transformed BCG-Tice and confirms the successful preparation of BCG-Tice-disA-OE by electroporation of the pSD5-hsp65-MT3692 plasmid into BCG-Tice. Note on nomenclature, the inventors had previously referred to this same plasmid pSD5-hsp60-MT3692. However, the actual promoter in this strain is the promoter for the hsp65 gene of M. leprae. Thus, the inventors now more correctly refer to the plasmid as pSD5-hsp65-MT3692.

FIG. 23 shows that clone 2 of BCG-Tice-disA-OE from the transformation experiment shown in FIG. 22 strongly expresses the disA gene. Real time PCR was used to show differential disA expression in four different BCG-Tice-disA-OE clones. Gene expression was measured in total RNA isolated from the late log phase cultures using log phase cultures using SYBR green based quantitative real-time PCR. The graphical data points represent the mean of 3 independent experiments ±standard error mean (SEM). M tuberculosis sigA (Rv2703) was used as an internal control. Data analysis was performed using 2^{-ΔΔCT} method. Student's t test followed by Welch correction (***P<0.001; **P<0.01). The inventors created seedlots of BCG-Tice-disA-OE clone 2, and refer to this clone as simply “BCG-Tice-disA-OE” in all subsequent work.

FIG. 24 shows potent, statistically significantly enhanced IRF3 induction in mouse bone marrow-derived macrophages infected with BCG-Pasteur-disA-OE compared with BCG-Pasteur-WT. Mouse (C57BL/6) bone marrow-derived macrophages were infected with wild-type and disA overexpressing strains of BCG Pasteur (20 MOI) for 3 h. Cells were washed with warm DPBS to remove non-internalized bacilli and were subsequently incubated for another 3 hours. IRF3 expression was measured in total RNA isolated from the cell lysate using SYBR green based quantitative real-time PCR. The graphical data points represent the mean of 3 independent experiments ±standard error mean (SEM). Mouse beta-actin was used as an internal control. Data analysis was performed using 2^{-ΔΔCT} method. Student's t test followed by Welch correction (***P<0.001; **P<0.01).

FIG. 25 shows that STING is required for enhanced Type I IFN (IFN-β) induction in response to BCG-WT and BCG-disA-OE. Mouse (C57BL/6) bone marrow-derived macrophages from STING ablated (STING-KO) wild-type animals were infected with different strains of BCG (MOI=1:20) for 3 h. Cell were washed using warm DPBS to removed non-internalized bacilli and were subsequently incubated in for another 24 h before culture supernatants were harvested. ELISA for IFN-β was performed in culture supernatants as per the manufacturer's instruction. Data points represent the mean of three independent biological experiments±standard error mean (S.E.M.). Student's t test followed by Welch correction (**P<0.01).

FIG. 26 shows that interferon-β is induced murine BMDMs, BMDCs and J774.1 macrophages in upon exposure to disA overexpressing BCG strains and that the IFN-β response is statistically significantly greater for BCG-Pasteur-disA-OE and BCG-Tice-disA-OE than for the corresponding BCG-WT strains. Mouse (C57BL/6) bone marrow-derived macrophages (BMDMs), and J774.1 macrophages were infected for 3 h using different strains of BCG (MOI: 20). Non-internalized bacilli were washed using warm DPBS and cell were incubated for another 24 hours. IFN-β levels were quantified in culture supernatants using ELISA as per manufacturer's instruction. Data points represent three independent biological experiments±standard error mean (S.E.M.). Data analysis was performed using unpaired t-test (***P<0.001; **P<0.01; *P<0.05).

FIG. 27 shows that IL-6 is induced in mouse BMDMs, BMDCs and J774.1 macrophages in response to exposure to disA overexpressing BCG strains and that the IL-6 response is statistically significantly greater for BCG-Pasteur-disA-OE and BCG-Tice-disA-OE than for the corresponding BCG-WT strains. Mouse (C57BL/6) bone marrow-derived macrophages (BMDMs), and J774.1 macrophages were infected for 3 h using different strains of BCG (MOI: 20). Non-internalized bacilli were washed using warm DPBS and cell were incubated for another 24 hours. IL-6 levels were quantified in culture supernatants using ELISA as per manufacturer's instruction. Data points represent three independent biological experiments±standard error mean (S.E.M.). Data analysis was performed using unpaired t-test (***P<0.001; **P<0.01; *P<0.05).

FIG. 28 shows that TNF is induced in mouse BMDMs, BMDCs and J774.1 macrophages in response to exposure to disA overexpressing BCG strains and that the responses are statistically significantly greater for BCG-Pasteur-disA-OE and BCG-Tice-disA-OE than for the corresponding BCG-WT strains. Mouse (C57BL/6) bone marrow-derived macrophages (BMDMs), and J774.1 macrophages were infected for 3 h using different strains of BCG (MOI: 20). Non-internalized bacilli were washed using warm DPBS and cell were incubated for another 24 hours. TNF levels were quantified in culture supernatants using ELISA as per manufacturer's instruction. Data points represent three independent biological experiments±standard error mean (S.E.M.). Data analysis was performed using unpaired t-test (***P<0.001; **P<0.01; *P<0.05).

FIG. 29 shows that TNF and IFN-γ are induced the in the rat bladder carcinoma NBT-II cell line in response to exposure to disA overexpressing BCG strains and that the two responses are statistically significantly greater for BCG-Pasteur-disA-OE and BCG-Tice-disA-OE than for the corresponding BCG-WT strains. NBT-II cells were infected with wild-type and recombinant strains of BCG for 3 h. Non-internalized bacilli were repeatedly washed using warm DPBS and cells were incubated for another 24 h. Culture supernatants were used for quantification of TNF and IFN-γ. Data points represent three independent biological experiments±standard error mean (S.E.M.). Data analysis was performed using unpaired t-test (***P<0.0001; **P<0.001; *P<0.05).

FIG. 30 shows that of IFN-62 , IFN-γ, TNF and IL-1β in are induced the in the human transitional cell papilloma RT4 bladder cancer cell line in response to exposure to disA overexpressing BCG strains and that the two responses are greater for BCG-Pasteur-disA-OE and BCG-Tice-disA-OE than for the corresponding BCG-WT strains. RT4 cells were infected with wild-type and recombinant strains of BCG for 3 h. Non-internalized bacilli were repeatedly washed using warm DPBS and cells were incubated for another 24 h. Culture supernatants were used for quantification of cytokines as per manufacturer's instruction. Data points represent two independent biological experiments±standard error mean (S.E.M.). Data analysis was performed using unpaired t-test (***P<0.001; **P<0.01; *P<0.05).

FIG. 31 shows that BCG-disA-OE stimulates increased IFN-β levels in multiple bladder cancer cell lines to a greater degree than BCG-WT. The drawing shows the levels of IFN-β mRNA (relative expression by the 2^{-ΔΔCT} method) following exposure to BCG-WT, BCG-disA-OE, and LPS. 5637 cells are human muscle-invasive bladder cancer cells, RT4 cells are human transitional cell papilloma bladder cancer cells, and NBT-II cells are rat bladder carcinoma cells induced by N-butyl-N-(-4-hydroxybutyl) nitrosamine.

FIG. 32 shows the cytokine responses for IFN-β, IFN-γ, IL-6, and TNF in BCG-WT and BCG-disA-OE-infected mouse lungs at different time points following aerosol infection. The drawing reveals that at most time points for most cytokines, the responses are greater for BCG-Pasteur-disA-OE and BCG-Tice-disA-OE than for the corresponding BCG-WT strains. BALB/c mice were infected by the aerosol route as described in FIG. 19. Groups of mice were sacrificed at 2, 4, and 6 weeks after infection. Lung homogenates were prepared, and cytokine levels were quantified using ELISA as per manufacturer's protocol (n=4 animals/treatment group±S.E.M.). Data analysis was performed using paired t-test (***P<0.001; **P<0.01; *P<0.05).

FIG. 33 shows the cytokine responses for IFN-β, IFN-γ, IL-6, and TNF in BCG-WT and BCG-disA-OE-infected mouse spleens at 4 weeks following aerosol infection. The drawing reveals that for most cytokines, the responses are greater for BCG-Pasteur-disA-OE and BCG-Tice-disA-OE than for the corresponding BCG-WT strains. BALB/c mice were infected by the aerosol route as described in FIG. 19. Groups of mice were sacrificed at 4 weeks after infection. Spleen homogenates were prepared, and cytokine levels were quantified using ELISA as per manufacturer's protocol (n=4 animals/treatment group±S.E.M.). Data analysis was performed using paired t-test (***P<0.001; **P<0.01; *P<0.05).

FIG. 34 shows a method to generate antibiotic-resistance gene-free recombinant BCG which overexpresses a STING-agonist. In the example given, a genetically-modified, antibiotic resistance gene-free BCG is created to overexpress the BCG disA gene and release excess c-di-AMP (a known STING agonist). However, the same strategy may be used to overexpress the Rv1354c (diguanylate cyclase that makes c-di-GMP another known STING agonist), DncV (cyclic GMP-AMP synthase from V. cholerae that makes 3′-3′-cGAMP another known STING agonist), cGAS (cyclic GMP-AMP synthase from humans that makes 2′-3′cGAMP another known STING agonist), or another similar enzyme gene that generates a STING agonist.

In Step 1 a BCG-WT strain is transformed by electroporation of the plasmid pJV53 (SEQ ID NO: 32) and selection on kanamycin-containing 7H11 agar plates. pJV53 harbors the gp60 and gp61 genes from the mycobacteriophages Che9c which encodes homologs of RecE and RecT, respectively. Che9c gp60 and gp61 encode exonuclease and DNA-binding activities, respectively, and expression of these proteins substantially elevates mycobacterial homologous recombination proficiency. Step 1 yields a “recombination proficient BCG” as shown in the drawing. After confirmation of transformants, the positive clones will be expanded in presence of kanamycin and will be used to make electrocompetent cells. To prepare these electrocompetent cells, bacteria are grown to mid-log phase, induced with 0.2% acetamide (to upregulate the expression of the Che9c gp60 and Che9c gp61 recombineering genes) for 24 hours prior to being made electrocompetent.

In Step 2, a linearized allelic exchange substrate (AES) construct corresponding to SEQ ID NO: 33 is generated. The AES (SEQ ID NO: 33) is comprised of the dif-Hyg-dif cassette (SEQ ID NO: 34) sequence flanked by 500 bp of the 5′UTR of the panCD operon on one side and 500 bp of the 3′UTR of the panCD operon on the other. The AES (SEQ ID NO: 33) is constructed by cloning 500 bp of the 5′UTR of the panCD operon and 500 bp of the 3′UTR of the panCD operon into pUC-Hyg plasmid (SEQ ID NO: 35) yielding the plasmid pUC-Hyg-panCD-KO (SEQ ID NO: 36). pUC-Hyg-panCD-KO (SEQ ID NO: 36) is cleaved by digestion with NruI and NcoI to yield the linear AES corresponding to SEQ ID

NO: 33. Alternatively, the primers SEQ ID NO: 28 and SEQ ID NO: 29 may be used to amplify the linear AES (SEQ ID NO: 33). The linearized allelic exchange substrate (AES) construct corresponding to SEQ ID NO: 33 is then electroporated into the “recombination proficient BCG”, and clones are selected on hygromycin and kanamycin-containing 7H11 agar plates. This step yields “BCG harboring the panCD KO cassette” in which the AES (SEQ ID NO: 33) has integrated into the panCD operon of the chromosome by homologous recombination.

In Step 3 several hundred colonies of “BCG harboring the panCD KO cassette” are replica plated on both (i) kanamycin-containing 7H11 agar plates and (ii) kanamycin-containing 7H11 agar supplemented with pantothenate (24 μg/ml). Kan-resistant, pantothenate auxotrophic clones are selected that only grow on kanamycin-containing 7H11 agar supplemented with pantothenate (24 μg/ml) and fail to grow on kanamycin-containing 7H11 agar plates lacking pantothenate. During this step the natural action of the mycobacterial Xer recombinase which recognizes and recombines at dif sites leads to excision and loss of the hygromycin cassette yielding clones which are “Pantothenate auxotrophs harboring pJV53” as shown.

In Step 4, “pantothenate auxotrophs harboring pJV53” are plated on 7H11 agar plates containing sucrose to select for loss of pJV53 which harbors the sacB gene (conferring lethality in the presence of sucrose). Sucrose-resistant clones are selected, and these are confirmed to be kanamycin-susceptible. This yield clones which are “Pantothenate auxotrophs free of pJV53”.

In Step 5, electrocompetent “Pantothenate auxotrophs free of pJV53” are prepared. The plasmid “pSD5.phsp65-disA.panCD-No Kan” (SEQ ID NO: 31) is generated as described in FIG. 36. SEQ ID NO: 31 is electroporated into “Pantothenate auxotrophs free of pJV53”, and clones are plated on 7H11 agar free of pantothenate to yield the desired “Pantothenate auxotroph harboring a disA-OE plasmid”. Candidate clones are confirmed by PCR of relevant genes and by whole genome sequencing.

FIG. 35 shows the molecular structure of the DNA fragment containing the panCD allelic exchange substrate (AES) which is SEQ ID NO: 33.

FIG. 36 shows the strategy used to generate “pSD5.hsp65-disA.panCD-No Kan” (SEQ ID NO: 31). The scheme replaces Kan cassette “pSD5.hsp65-disA.Kan” (SEQ IN NO: 30) with the panCD operon to generate “pSD5.hsp65-disA.panCD-No Kan” (SEQ ID NO: 31).

FIG. 37 shows the molecular structure of the pJV53, the recombineering plasmid which is SEQ ID NO: 32

FIG. 38 shows the molecular structure of the pUC-Hyg, a plasmid with dif sites flanking a Hyg cassette which is SEQ ID NO: 35. pUC-Hyg is used to generate the plasmid “pUC-Hyg-panCD-KO” (SEQ ID NO: 36).

FIG. 39 shows the molecular structure of the plasmid “pUC-Hyg-panCD-KO” which is SEQ ID NO: 36. “pUC-Hyg-panCD-KO” is generated by cloning 500 bp of the panCD 5′UTR on one flank of the Hyg cassette, and cloning 500 bp of the panCD 3′UTR the other flank.

FIG. 40 shows the molecular structure of the plasmid “pSD5.hsp65-disA.Kan” which is SEQ ID NO: 30.

FIG. 41 shows the molecular structure of the plasmid “pSD5.hsp65-disA.panCD-No Kan” which is SEQ ID NO: 31. This plasmid is generated using the scheme illustrated in FIG. 36.

FIG. 42 shows some of the nucleic acid and protein sequences used in the present invention.

FIG. 43 shows a description of the nucleic acid and protein sequences used in the present invention.

FIG. 44 shows the number of positive specimens.

### DETAILED DESCRIPTION OF THE INVENTION

In some embodiments, the present invention relates to genetic alterations of Mycobacterium bovis BCG (hereafter, “BCG”) which generate recombinant BCG (hereafter “rBCG”) strains. These strains have greater potency as (i) tuberculosis vaccines and/or (ii) immunotherapies for non-muscle invasive bladder cancer (NMIBC). Some embodiments of the present invention relate to BCG strains that synthesize and secrete high levels of cyclic dinucleotides (CDNs) which are known to elicit valuable immunomodulatory responses from human phagocytic cells such as macrophages, dendritic cells, and others. Another embodiment of this invention is to combine genetic modifications of BCG to generate multivalent CDN-overexpression modifications that include addition of novel CDN-synthesizing genetic material and/or mutations of endogenous BCG phosphodiesterase genes or genetic domains that will enhance the accumulation and release of CDNs.

BCG

BCG (bacillus Calmette Guerin) is a mutant version of Mycobacterium bovis generated by the French microbiologists Calmette and Guerin in 1921 by 13 years of serial passage of virulent M bovis. Between 1921 and 1960 BCG was carried by serial passage in numerous world laboratories, until defined seedlots were established and banked in reference laboratories. As such, many dozen variants of BCG exist worldwide such as BCG Pasteur, BCG Tice, BCG Tokyo, BCG Danish, BCG Montreal, etc. The majority of existing BCG strains have now been defined by whole genome sequencing. Major differences between virulent M. bovis and the various BCG strains include the deletion of at least 15 regions of difference that comprise genomic deletions in BCG compared with virulent M tuberculosis. Key regions of difference in the development of BCG were RD1 (9.5 kb deletion leading to loss of the Esx-1 secretion system and inability to release antigens ESAT-6 and CFP-10) and RD3 (9.2 kb deletion). Regions of difference RD4-RD11 are absent in all BCG strains compared with virulent M tuberculosis.

Since the 1920s, BCG has been used as a vaccine for prevention of tuberculosis (TB). In 2004 it was estimated that BCG was given to about 100 million children, hence since its introduction BCG has been given to approximately 5 billion humans and as such is the most widely utilized vaccine in history. It is most commonly given intradermally at birth, and to date it is still given in most countries except the United States, Canada, and parts of Europe. BCG has been shown to reduce the incidence of childhood disseminated TB, but BCG-vaccinated individuals are not fully protected from the risk of TB.

Since 1977, BCG has also achieved wide use as a cancer immunotherapy for non-muscle invasive bladder cancer (NMIBC). It is given intravesically weekly for six weeks and in some instances such as high-risk disease it is given as maintenance therapy weekly for three weeks at 3, 6, 12, 18, 24, 30, and 36 months after initial therapy. Intravesical BCG has been shown to (i) induce a mononuclear cell infiltrate comprised predominantly of CD4 T cells and macrophages, (ii) increase the expression of interferon gamma (IFNγ) in the bladder, and (iii) increase urinary cytokine levels of IL-1, IL-2, IL-6, IL-8, IL-12, IFNγ, and TNFα.

Despite the wide global use of BCG as (i) a vaccine for TB and (ii) an immunotherapy for NMIBC, there is considerable room for improvement in its efficacy. For TB, BCG gives only partial protection predominantly against childhood disseminated tuberculosis. For NMIBC, approximately 30% of patients have BCG-resistant disease. These individuals require riskier treatments with systemic chemotherapy and have higher rates of progression to more invasive forms of bladder cancer.

Urothelial Cancer

Urothelial cancer of the bladder is the most common malignancy of the urinary tract. It is the fourth most common cancer in males and 11th most common in females. It is estimated that approximately 79,000 new cases of bladder cancer will be diagnosed in the USA in 2017, associated with 19,870 deaths. Although the estimated five-year survival for bladder cancer patients is 78%, the rates decline dramatically for patients with locally advanced or metastatic disease. Approximately 75% of patients with bladder cancer present with a disease that is confined to the mucosa (stage Ta, carcinoma in situ) or submucosa (stage T1), know) as non-muscle invasive bladder cancer (NMIBC). Transurethral resection is the initial treatment of choice for NMIBC. For patients with muscle invasive bladder cancer (MIBC; T2 or greater), the first-line treatment option is platinum-containing chemotherapy followed by bladder removal. For those patients with NMIBC who do not respond to intravesical treatments, there is high risk of progression to MIBC. Thus, the high rates of recurrence and significant risk of progression mandate that additional therapy be implemented. Improving clinical outcomes for patients with high risk-NMIBC therefore requires the development of novel treatments.

Intra-vesical administration of Bacillus-Calmette Guerin (BCG), developed in the 1970s for NMIBC, provided the first successful immunotherapy against an established solid cancer, and it remains the standard of care for patients with NMIBC. (Askeland E J, Newton M R, O'Donnell M A, Luo Y. Bladder Cancer Immunotherapy: BCG and Beyond. Adv Urol. 2012; 2012:181987. PMID: 22778725. Morales A. BCG: A throwback from the stone age of vaccines opened the path for bladder cancer immunotherapy. Can J Urol. 2017; 24:8788-8793. PMID: 28646932). The exact mechanism of the anti-tumor effects of BCG, which is an attenuated strain of Mycobacterium bovis, remain unclear, but it is believed to orchestrate a vigorous immune cellular and humoral immune response, predominantly Th1 response, after binding to the urothelium through fibronectin and integrin a5131 (Redelman-Sidi G, Glickman M S, Bochner B H. The mechanism of action of BCG therapy for bladder cancer—a current perspective. Nat Rev Urol. 2014; 11:153-62. PMID: 24492433). However, typical complete response rates for BCG treatment are 55-65% for papillary tumors and 70-75% for carcinoma in situ (CIS). (Askeland E J, Newton M R, O'Donnell M A, Luo Y. Bladder Cancer Immunotherapy: BCG and Beyond. Adv Urol. 2012;2012:181987. PMID: 22778725. Morales A. BCG: A throwback from the stone age of vaccines opened the path for bladder cancer immunotherapy. Can J Urol. 2017; 24:8788-8793. PMID: 28646932). The burden of patients with BCG unresponsive and relapsing disease and of those intolerant of treatment has therefore prompted the need for further improving the efficacy of BCG against NMIBC.

CDNs are Important PAMPs and DAMPs that Generate Valuable Immune Responses for TB and NMIBC.

Bacterial pathogen-associated molecular patterns (PAMPs). Human cells utilize an innate immune monitoring system known as the cytosolic surveillance program (CSP) to detect nucleic acid including cyclic dinucleotides in the cytosol. Originally characterized as a viral defense system, the CSP has now been shown to be important in anti-bacterial defenses particularly against intracellular bacteria such as Mycobacterium tuberculosis, Listeria monocytogenes, Salmonella species, and others. Cytosolic pattern recognition receptors (PRRs) including STING, cGAS, DDX41 and many others are capable of binding to cytosolic CDNs and nucleic acids leading to their activation. A key signaling event is STING activation which leads to activation of TBK1 and IRF3 and subsequent upregulation of type I interferon expression. STING activation by cyclic dinucleotides also leads to the induction of STAT6 which induces chemokines such as CCL2 and CCL20 independently of the TBK1-IRF3 pathway. STING activation is also believed to activate the transcription factor NFκB through the κB kinase (IKK) activation.

Human danger associated molecular patterns (DAMPs). Cyclic cGAMP (cGAS) synthase is a cytosolic PRR which recognized cytosolic DNA. Upon binding to DNA it undergoes a conformational change that activates its core enzymatic activity which is to catalyze the formation of 2′3′ cGAMP. 2′3′ cGAMP in turn is a potent DAMP which activates the STING-TBK1-IRF3 axis leading to increased type 1 interferon expression as well as the STAT6 activation and IKK activation.

STING-mediated mechanism of CDN-triggered immune responses. Type I IFNs, produced both by innate immune cells in the tumor microenvironment and by the tumor cells themselves, are known to mediate anti-tumor effects against several malignancies, due to their ability to intervene in all phases of cancer immune-editing. (Zitvogel L, Galluzzi L, Kepp O, Smyth M J, Kroemer G. Type I interferons in anticancer immunity. Nat Rev Immunol. 2015;15:405-14. PMID: 26027717). STING (stimulator of interferon genes), is a major regulator of Type I IFN innate immune responses to pathogens, following recognition of cytosolic DNA by the sensor cyclic GMP-AMP synthase (cGAS). cGAS catalyzes the synthesis of cyclic GMP-AMP (cGAMP), which in turn functions as a second messenger that binds and activates STING. (Zhao G N, Jiang D S, Li H. Interferon regulatory factors: at the crossroads of immunity, metabolism, and disease. Biochim Biophys Acta. 2015;1852:365-78. PMID: 24807060). Novel anticancer immunotherapies based on recombinant type I IFNs, type I IFN-encoding vectors, type I IFN-expressing cells, and STING agonists are therefore currently being developed as novel tumor immunotherapies.

Overexpression of the PAMP immunomodulator, 3′-5′ c-di-AMP. 3′-5′ c-di-AMP is a strong inducer of the STING-TBK1-IRF3 axis. It is produced by mycobacteria including BCG by the disA gene which encodes the DisA protein (BCG protein WP 010950916.1 in BCG, M tuberculosis protein Rv3586 or P9WNW5.1). Mycobacterium tuberculosis (M.tb) synthesizes and secretes c-di-AMP, which activates the interferon regulatory factor (IRF) pathway and Type I IFN responses through STING-signaling and cGAS. (Ahmed D, Cassol E. Role of cellular metabolism in regulating type I interferon responses: Implications for tumour immunology and treatment. Cancer Lett. 2017; 409:20-29. PMID: 28888999.). c-di-AMP overexpressing M. tb strains showed attenuation of TB in a mouse model. As a mucosal adjuvant, c-di-AMP exerts immune stimulatory effects causing maturation of dendritic cells, up-regulation of co-stimulatory molecules and production of pro-inflammatory cytokines, and strong Th1, Th17 and CD8 T cell responses against pathogens. A c-di-AMP-overexpressing BCG strain (rBCG-disA or BCG-disA-OE) has been constructed and surprisingly found that it produced a significantly higher IRF and IFN-β response than BCG itself, indicating that bacteria-derived c-di-AMP gains access to the host cell cytosol despite the absence of the ESX-1 protein secretion system. (Ahmed D, Cassol E. Role of cellular metabolism in regulating type I interferon responses: Implications for tumour immunology and treatment. Cancer Lett. 2017; 409:20-29. PMID: 28888999.). These findings suggest that rBCG strains modified to overexpress c-di-AMP could induce better protective immunity against bladder tumors than BCG itself.

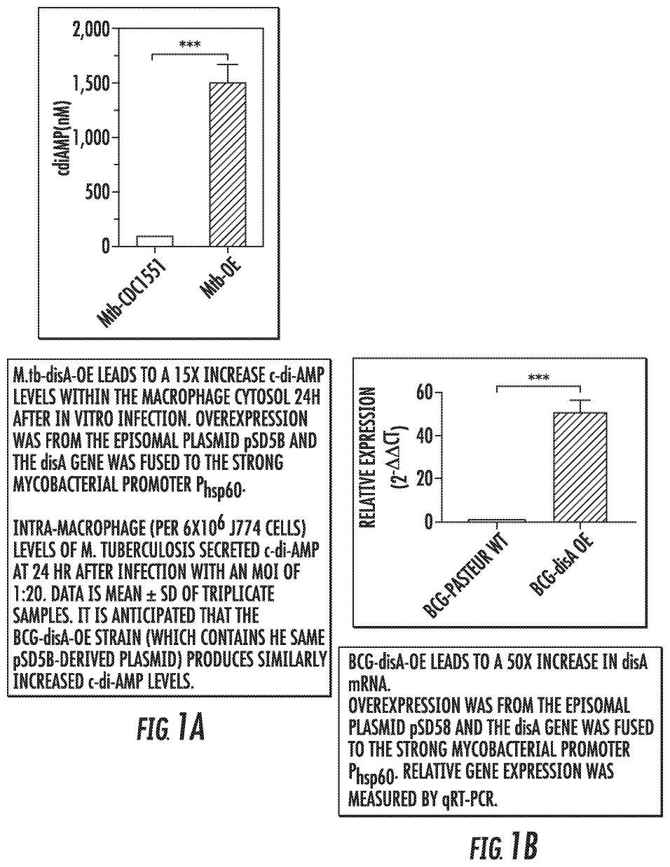

Induction of pro-inflammatory Th1 cytokines in mouse bone marrow-derived macrophages (BMDMs) in response to BCG overexpressing M.tb disA (MT3692): M.tb genome encodes a di-adenylate cyclase enzyme (DisA, also called DacA, P9WNW5.1 in the UniProtKB/Swiss-Prot databases) that synthesizes c-di-AMP from ATP or ADP. The BCG protein WP_010950916.1 (NCBI reference number) is 100% identical to M. tuberculosis DisA. M. tb strains overexpressing disA intoxicate macrophages by releasing excessive c-di-AMP, a unique bacterial PAMP that activates STING-dependent IFN-βproduction. (Ahmed D, Cassol E. Role of cellular metabolism in regulating type I interferon responses: Implications for tumour immunology and treatment. Cancer Lett. 2017; 409:20-29. PMID: 28888999.). To expand the antigenic repertoire of a non-pathogenic vaccine strain, BCG Pasteur was transformed with a kanamycin-resistance (Kan-R)-conferring plasmid that harbors the disA gene (M tuberculosis Rv3586 or MT3692) from M. tb (the M. tb and BCG disA genes are 100% identical) fused to the strong mycobacterial promoter, Phsₚ₆₀. Addition of this plasmid to BCG-Pasteur increased the level of disA mRNA by 50-fold (FIG. 1b). The closely related M.tb-disA-OE strain releases 15-fold more c-di-AMP into the macrophage cytosol than wild type M. tb. (FIG. 1a), and hence it is expected that BCG-disA-OE also releases significantly more c-di-AMP into the host cytosol. These disA overexpressor recombinants (rBCG or BCG-disA-OE) were better inducers of STING-dependent IFN-β as compared to the parental strain. Most importantly as reported in PCT/US2016/017248, filed Feb. 10, 2016, guinea pigs vaccinated with rBCG were significantly better protected against aerosol infection with virulent M.tb, suggesting improved protective efficacy over existing BCG strain.

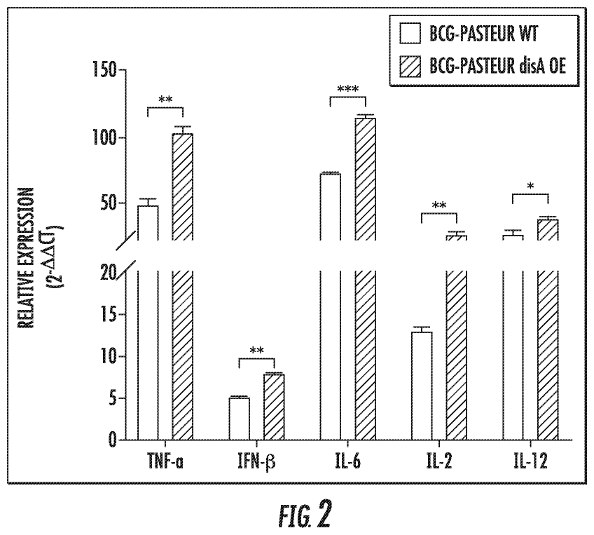

As shown in FIG. 2, immune responses elicited by BCG-Pasteur disA-OE were tested in an in vitro macrophage infection model. BMDMs from C57BL/6 mice infected with BCG-Pasteur disA-OE showed significant upregulation of IFN-β, TNF-α, IL-6 and IL-2 in comparison to uninfected or wild-type BCG infected macrophages.

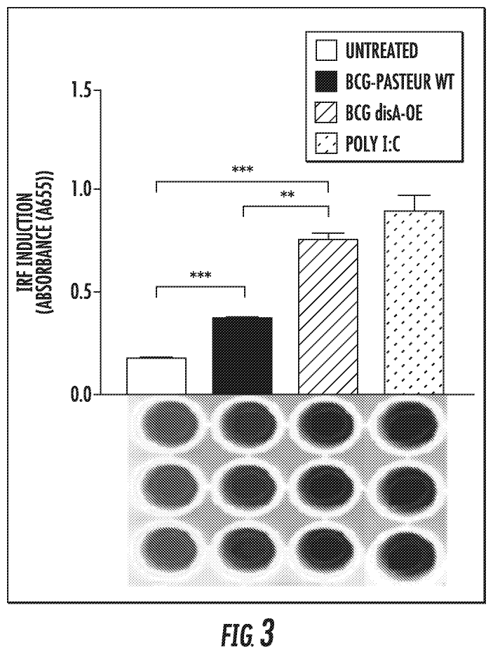

As shown in FIG. 3, augmented c-di-AMP-based STING activation was confirmed in RAWBlue ISG macrophages. RAWBlue macrophages showed increased IRF3 levels when infected with BCG-Pasteur disA-OE, as compared to parental control.

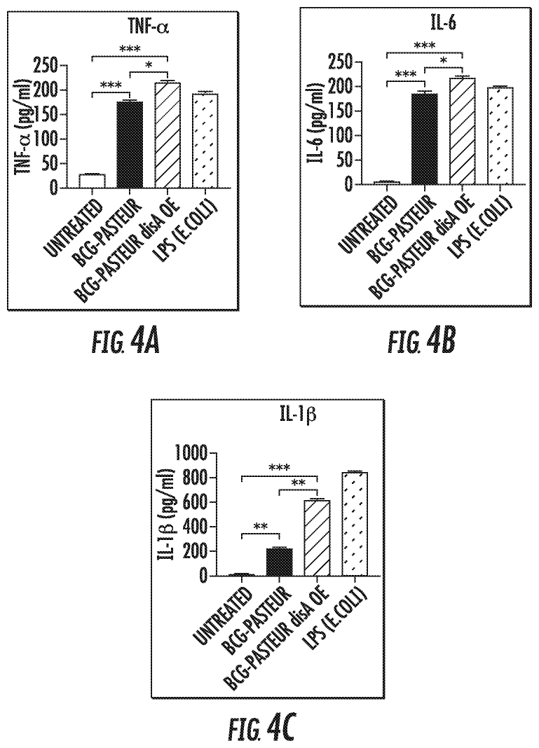

As shown in FIG. 4, a significant increase in secreted pro-inflammatory cytokines (TNF-α, IL-6 and IL-1β) was found in culture supernatants of BCG-Pasteur-disA-OE infected mouse BMDMs. These findings indicate that BCG-Pasteur-disA-OE with increased antigenic repertoire acts like a STING agonist, and hence a potent inducer of STING-dependent Type I IFNs. Furthermore, the immune responses in macrophages in response to BCG-Pasteur disA-OE were skewed towards Th1, a phenotype largely attributed for control of NMIBC by BCG immunotherapy.

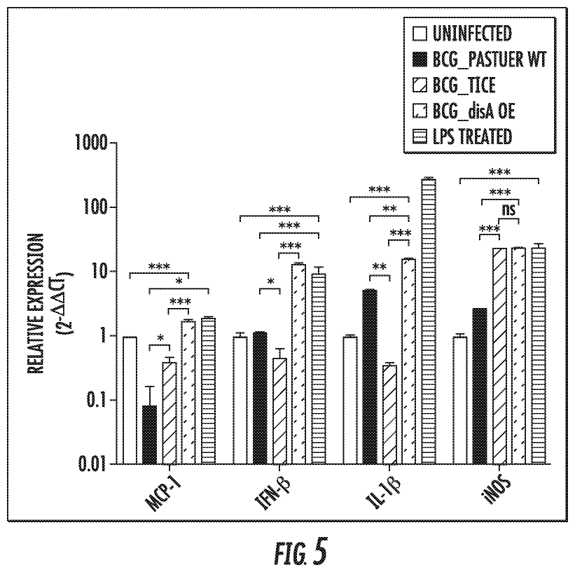

As shown in FIG. 5, BCG-disA-OE elicits anti-tumor immune responses in human bladder carcinoma (RT4) cells. BCG-Pasteur-disA-OE was tested to determine whether it elicits similar immune responses in bladder cancer (BC) cells, in comparison to WT strains BCG-Pasteur and OncoTICE (the current immunotherapeutic BCG strain). Human RT4 BC cells, derived from human NMIBC tumors, were challenged with the wild-type (both Pasteur and TICE) and recombinant BCG Pasteur disA-OE strain at 1:20 (RT4::BCG) for 3 h, and differential gene expression profile was determined in comparison to uninfected cells. Key immune mediators such as, monocyte chemoattractant protein 1 (MCP-1)/CCL2, IFN-β and IL-1β were found to be significantly increased in bladder cancer cells exposed to BCG-Pasteur-disA-OE compared to responses to wild type strains.

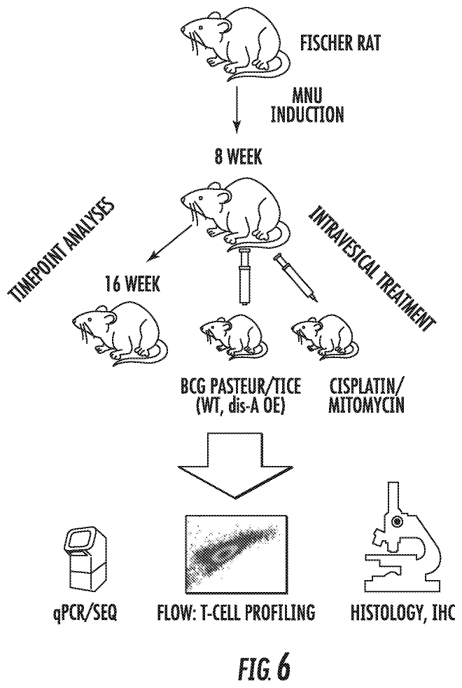

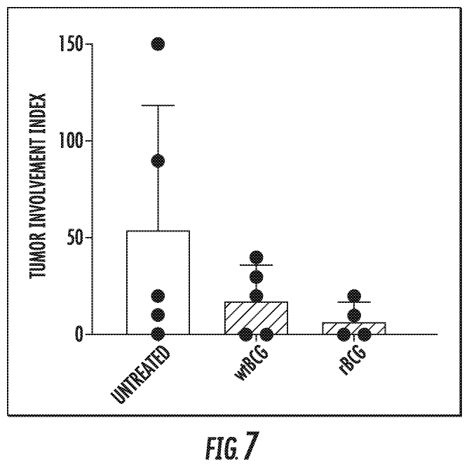

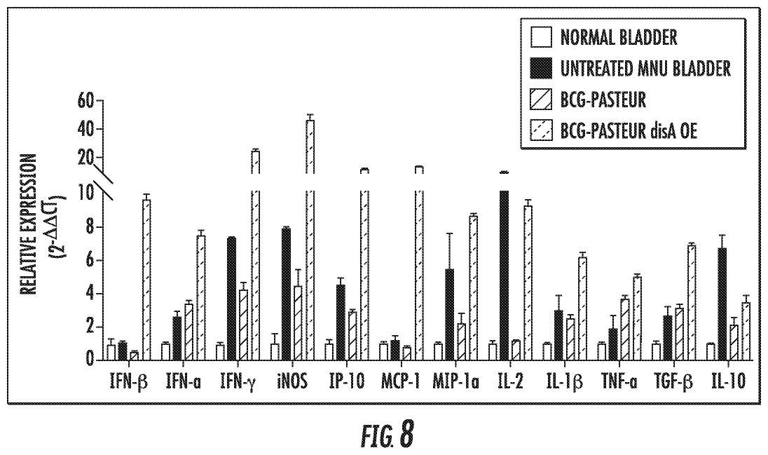

As shown in FIG. 6, an experimental system was set up to test whether intravesical BCG-disA-OE immunotherapy leads to heightened Th1 responses and anti-tumor efficacy in the MNU carcinogen model of NMIBC. Results from the aforementioned experiments with RT4 cells encouraged the inventors to test the relative therapeutic efficacy of BCG-Pasteur disA OE in an in vivo rat NMIBC model, pioneered in Bivalacqua lab. (Kates M, Nirschl T, Sopko N A, Matsui H, Kochel C M, Reis L O, Netto G J, Hoque M O, Hahn N M, McConkey D J, Baras A S, Drake C G, Bivalacqua T J. Intravesical BCG Induces CD4(+) T-Cell Expansion in an Immune Competent Model of Bladder Cancer. Cancer Immunol Res. 2017; 5:594-603. PMID: 28588015). In this model, N-methyl-N-nitrosourea (MNU), a carcinogenic alkylating agent, is used to induce urothelial cancer in female Fischer rats.

As can be seen in FIG. 7, BCG-disA-OE has significant immunotherapeutic effects in the rat bladder cancer model. Urothelial dysplasia develops within eight weeks of MNU instillation, and by the 16th week after the first instillation, all rats display carcinoma-in-situ, papillary Ta, or high-grade T1 urothelial carcinoma with histopathologic and immunophenotypic features similar to those observed in human urothelial cancer. Using this model, the Bivalacqua lab showed that intravesical BCG immunotherapy lead to a large, transient rise in the CD4⁺ T cell population in the urothelium. (Kates M, Nirschl T, Sopko N A, Matsui H, Kochel C M, Reis L O, Netto G J, Hoque M O, Hahn N M, McConkey D J, Baras A S, Drake C G, Bivalacqua T J. Intravesical BCG Induces CD4(+) T-Cell Expansion in an Immune Competent Model of Bladder Cancer. Cancer Immunol Res. 2017;5:594-603. PMID: 28588015). Intravesical instillation of BCG-disA-OE strain was performed in MNU-treated rats, administered sequentially every week for 6 weeks starting eight weeks after MNU induction when tumors are visible. Bladder tumors were staged by a GU pathologist according to WHO-ISUP classifications with percent tumor involvement (sum of Ta, T1 and CIS) calculated for each group according to criteria as described. (Kates M, Nirschl T, Sopko N A, Matsui H, Kochel C M, Reis L O, Netto G J, Hoque M O, Hahn N M, McConkey D J, Baras A S, Drake C G, Bivalacqua T J. Intravesical BCG Induces CD4(+) T-Cell Expansion in an Immune Competent Model of Bladder Cancer. Cancer Immunol Res. 2017;5:594-603. PMID: 28588015). A significant decrease in tumor involvement index in rats treated with BCG-Pasteur disA-OE was found in comparison to bladders from untreated or BCG-Pasteur treated rats.

As can be seen in FIG. 8, BCG-disA-OE induces a characteristic cytokine and chemokine signature in rat bladders undergoing immunotherapy. Rat urinary bladders from rats treated with BCG-disA-OE showed a significant induction of IFN-α/β, IFN-γ, IL-1β, TNF-α, TGF-β, iNOS, IP-10, MCP-1 and MIP-la in comparison to untreated or BCG-Pasteur treated rats.

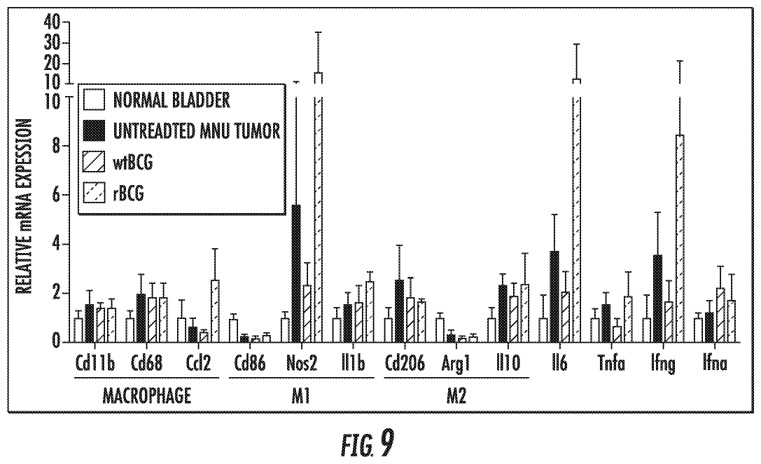

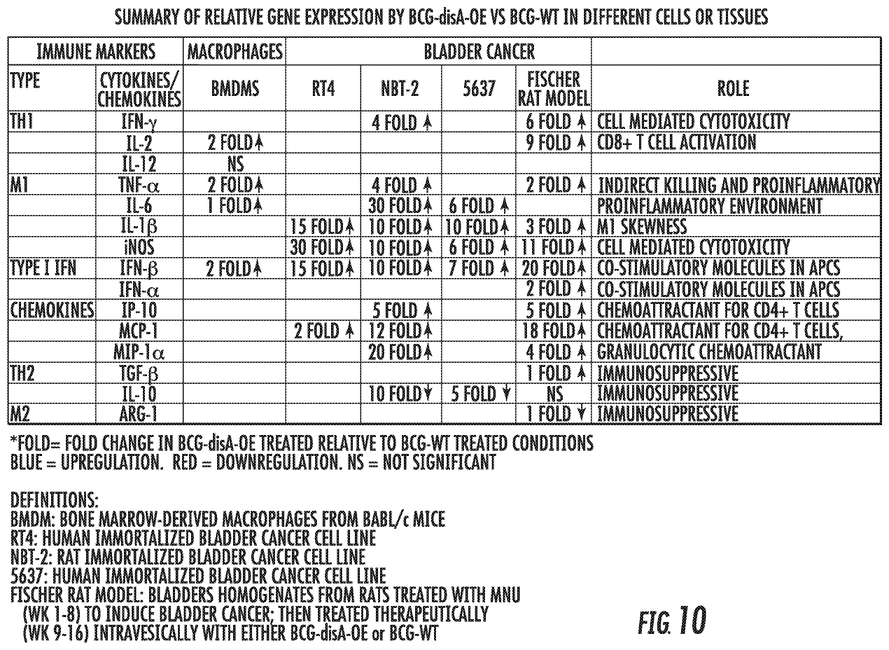

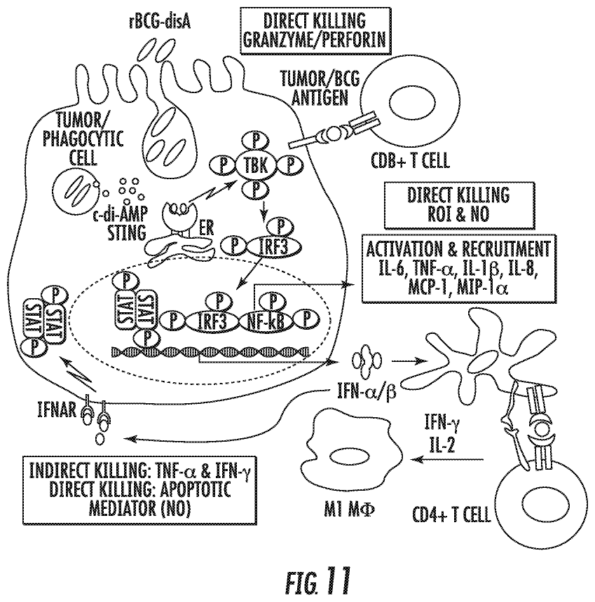

As shown in FIG. 9, evidence was found for increased infiltration of CCL2⁺ macrophages, Nos2⁺ and IL-1β⁺ M1 macrophages, accompanied by increased IL-6 and IFN-expression in bladders of rats treated with BCG-Pasteur-disA-OE. Interestingly, increased levels of IP-10 were found, which together with increased IFN-γ is known to promote a strong T cell recruitment at the site of infection and inflammation.

FIG. 10 shows a summary of the cytokine expression level changes observed with BCG-disA-OE versus BCG-WT in primary cells, cancer cell lines, and in rat bladder cancer tissues. As can be seen, cytokines associated with Th1 T cell and M1 macrophage expansion, two Type 1 interferons, and three pro-inflammatory chemokines were significantly unregulated by BCG-disA-OE compared to BCG-WT (2-fold to 30-fold) across these cells, cell lines and tissues. In contrast, cytokines associated with Th2 T cell and M2 macrophage expansion were generally down-regulated by BCG-disA-OE in comparison to BCG-WT (1-fold to 10-fold).

As shown in FIG. 11, BCG immunotherapy may be effective via three immune mechanisms: (i) increased generation of tumor-specific cytotoxic CD8 T cells, (ii) cytokine environment which promotes macrophage-mediated CD4 cell activation against tumor antigens, and (iii) macrophage M1 shift promoting enhanced tumoricidal activity. The findings reported herein strongly indicate that BCG overexpressing c-di-AMP is taken up by bladder tumor cells, and myeloid cells that are either resident or recruited to the tumor microenvironment, and induces host immune responses, including activation of STING and Type I IFN responses, and NF-KB signaling, that promotes secretion of cytokines and chemokines, macrophage recruitment and apoptotic mechanisms, all of which collectively reduce tumor progression.

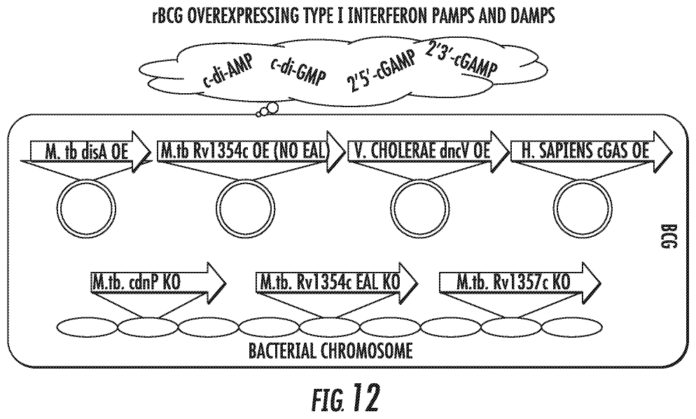

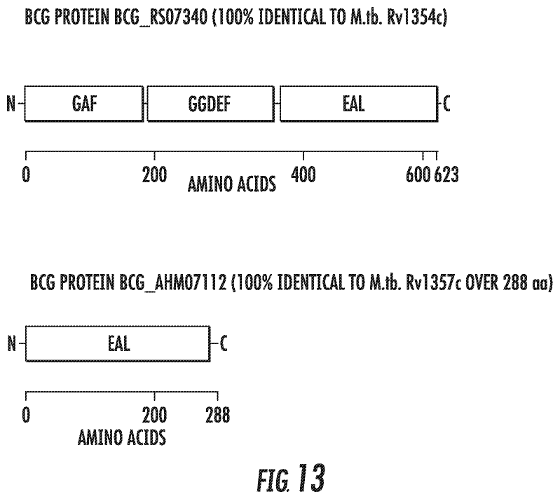

As shown in FIG. 12, in addition to overexpression of disA generating increased levels of the PAMP molecule c-di-AMP, there are additional recombinant DNA modification which may be made to BCG to enhance its production of other PAMP and DAMP molecules. As shown in the FIG. 12, genes for other CDN cyclases—(i) the GGDEF domain of the BCG_RS07340 protein orM tuberculosis Rv1354c protein (100% identical to each other), (ii) the Vibrio cholerae DncV protein, Q9KVG7 in Swiss-Prot, which is a 2′-5′c-GAMP synthase, and (iii) the human cGAS protein Q8N884 in Swiss-Prot which is a 2′-3′ cGAMP synthase—may be added to BCG. These added CDN cyclase genes may be added alone or in combination. Such combinations would represent multivalent CDN overexpressing BCG. Also, as shown in FIG. 13, BCG possess several CDN phosphodiesterase genes or genes which contain phosphodiesterase domains. Recombinant technology methods to remove these endogenous phosphodiesterase genes and intragenic phosphodiesterase domains: (i) the BCG WP_003414507 gene which encodes a CDN PDE in BCG that is 100% identical to the M. tuberculosis Rv2837c (also called CdnP or CnpB), (ii) the DNA encoding the EAL domain of protein BCG_RS07340 (previously BCG_1416c) which is 100% identical to the known CDN PDE M. tuberculosis Rv1354c protein, and (iii) the gene encoding BCG AHM07112 which is homologous the known CDN PDE M. tuberculosis Rv1357c. Removal of the genes encoding these PDEs will serve to further increase the levels of CDN PAMP and DAMP molecules produced by the rBCG strains disclosed herein.

SEQ ID NO: 1

Diadenylate cyclase DisA from BCG and other related mycobacteria, amino acid sequence (358 amino acids; BCG protein A0Q92 RS18745; NCBI Reference Sequence: NZ_CUWL01000001.1). The identical sequence is present in other strains of BCG, e.g., Mycobacterium tuberculosis as protein Rv3586 or MT3692, and in Mycobacterium bovis as protein Mb3617.

MHAVTRPTLREAVARLAPGTGLRDGLERILRGRTGALIVLGHDENVEAIC
DGGFSLDVRYAATRLRELCKMDGAVVLSTDGSRIVRANVQLVPDPSIPTD
ESGTRHRSAERAAIQTGYPVISVSHSMNIVTVYVRGERHVLTDSATILSR
ANQAIATLERYKTRLDEVSRQLSRAEIEDFVTLRDVMTVVQRLELVRRIG
LVIDYDVVELGTDGRQLRLQLDELLGGNDTARELIVRDYHANPEPPSTGQ
INATLDELDALSDGDLLDFTALAKVFGYPTTTEAQDSTLSPRGYRAMAGI
PRLQFAHADLLVRAFGTLQGLLAASAGDLQSVDGIGAMWARHVREGLSQL
AESTISDQ

SEQ ID NO: 2

Diadenylate cyclase disA from BCG and other related mycobacteria, DNA sequence (1077 nucleotides [358 codons, 1 stop codon]; encodes BCG gene A0Q92_RS18745; NCBI Reference Sequence: NZ_CUWL01000001.1) Identical sequence is present in other strains of BCG, e.g., Mycobacterium tuberculosis as gene Rv3586 or MT3692, Mycobacterium bovis as gene Mb3617.

| | |
|---|---|
| 1 | atgcacgctg tgactcgtcc gaccctgcgt gaggctgtcg cccgcctagc cccgggcact |
| 61 | gggctgcggg acggcctgga gcgtatcctg cgcggccgca ctggtgccct gatcgtgctg |
| 121 | ggccatgacg agaatgtcga ggccatctgc gatggtggct tctccctcga tgtccgctat |
| 181 | gcagcaaccc ggctacgcga gctgtgcaag atggacggcg ccgtggtgct gtccaccgac |
| 241 | ggcagccgca tcgtgcgggc caacgtgcaa ctggtaccgg atccgtcgat ccccaccgac |
| 301 | gaatcgggga cccggcaccg ctcggccgag cgggccgcga tccagaccgg ttacccggtg |
| 361 | atctcagtga gccactcgat gaacatcgtg accgtctacg tccgcgggga acgtcacgta |
| 421 | ttgaccgact cggcaaccat cctgtcgcgg gccaaccagg ccatcgcaac cctggagcgg |
| 481 | tacaaaacca ggctcgacga ggtcagccgg caactgtcca gggcagaaat cgaggacttc |
| 541 | gtcacgctgc gcgatgtgat gacggtggtg caacgcctcg agctggtccg gcgaatcggg |
| 601 | ctggtgatcg actacgacgt ggtcgaactc ggcactgatg gtcgtcagct gcggctgcag |
| 661 | ctcgacgagt tgctcggcgg caacgacacc gcccgggaat tgatcgtgcg cgattaccac |
| 721 | gccaacccgg aaccaccgtc cacggggcaa atcaatgcca ccctggacga actggacgcc |
| 781 | ctgtcggacg gcgacctcct cgatttcacc gcgctggcaa aggttttcgg atatccgacg |
| 841 | accacggaag cgcaggattc ggcgctgagc ccgcgtggct accgcgcgat ggccggtatc |
| 901 | ccccggctcc agttcgccca tgccgacctg ctggtccggg cgttcggaac gttgcagggt |
| 961 | ctgctggcgg ccagcgccgg cgatctgcaa tcagtggacg gcatcggcgc catgtgggcc |
| 1021 | cgtcatgtgc gcgatgggtt gtcacagctg gcggaatcga ccatcagcga tcaataa |

SEQ ID NO: 3.

Plasmid pSD5B-Pₕₛₚ₆₀::disA which is an episomally replicating E. coli-mycobacterial shuttle plasmid that overexpresses the BCG disA gene from the Pₕₛₚ₆₀ promoter, DNA sequence. (7742 nucleotides; promoter Pₕₛₚ₆₀ DNA comprised of a portion of the M. leprae hsp65 gene nucleotides 13 to 181) is underlined; disA coding sequence nucleotides 242 to 1318; ATG start codon and TAA stop codon shown in boldface, underline).

| | |
|---|---|
| GGATCCTTCTAGAATTCCGGAATTGCACTCGCCTTAGGGGAGTGCTAAAAATGATCCTGGCACTCGCGATCAGCGAG | 1-77 |
| TGCCAGGTCGGGACGGTGAGACCCAGCCAGCAAGCTGTGGTCGTCCGTCGCGGGCACTGCACCCGGCCAGCGTAAGT | 78-154 |
| AATGGGGGTTGTCGGCACCCGGTGACCTAGACACATGCATGCATGCTTAATTAATTAAGCGATATCCGGAGGAATCA | 155-231 |
| CTTCCATATGATGCACGCTGTGACTCGTCCGACCCTGCGTGAGGCTGTCGCCCGCCTAGCCCCGGGCACTGGGCTGC | 232-308 |
| GGGACGGCCTGGAGCGTATCCTGCGCGGCCGCACTGGTGCCCTGATCGTGCTGGGCCATGACGAGAATGTCGAGGCC | 309-385 |
| ATCTGCGATGGTGGCTTCTCCCTCGATGTCCGCTATGCAGCAACCCGGCTACGCGAGCTGTGCAAGATGGACGGCGC | 386-462 |
| CGTGGTGCTGTCCACCGACGGCAGCCGCATCGTGCGGGCCAACGTGCAACTGGTACCGGATCCGTCGATCCCCACCG | 463-539 |
| ACGAATCGGGGACCCGGCACCGCTCGGCCGAGCGGGCCGCGATCCAGACCGGTTACCCGGTGATCTCAGTGAGCCAC | 540-616 |
| TCGATGAACATCGTGACCGTCTACGTCCGCGGGGAACGTCACGTATTGACCGACTCGGCAACCATCCTGTCGCGGGC | 617-693 |
| CAACCAGGCCATCGCAACCCTGGAGCGGTACAAAACCAGGCTCGACGAGGTCAGCCGGCAACTGTCCAGGGCAGAAA | 694-770 |
| TCGAGGACTTCGTCACGCTGCGCGATGTGATGACGGTGGTGCAACGCCTCGAGCTGGTCCGGCGAATCGGGCTGGTG | 771-847 |
| ATCGACTACGACGTGGTCGAACTCGGCACTGATGGTCGTCAGCTGCGGCTGCAGCTCGACGAGTTGCTCGGCGGCAA | 848-924 |
| CGACACCGCCCGGGAATTGATCGTGCGCGATTACCACGCCAACCCGGAACCACCGTCCACGGGGCAAATCAATGCCA | 925-1001 |
| CCCTGGACGAACTGGACGCCCTGTCGGACGGCGACCTCCTCGATTTCACCGCGCTGGCAAAGGTTTTCGGATATCCG | 1002-1078 |
| ACGACCACGGAAGCGCAGGATTCGACGCTGAGCCCGCGTGGCTACCGCGCGATGGCCGGTATCCCCCGGCTCCAGTT | 1079-1155 |
| CGCCCATGCCGACCTGCTGGTCCGGGCGTTCGGAACGTTGCAGGGTCTGCTGGCGGCCAGCGCCGGCGATCTGCAAT | 1156-1232 |
| CAGTGGACGGCATCGGCGCCATGTGGGCCCGTCATGTGCGCGAGGGGTTGTCACAGCTGGCGGAATCGACCATCAGC | 1233-1309 |
| GATCAATAAACGCGTTCTGGCGTAATAGCGAAGAGGCCCGCACCGATCGCCCTTCCCAACAGTTGCGCAGCCTGAAT | 1310-1386 |
| GGCGAATGGCGCTTTGCCTGGTTTCCGGTCGAAGCTTGGCCGGATCTAAAGTTTTGTCGTCTTTCCAGACGTTAGTA | 1387-1463 |
| AATGAATTTTCTGTATGAGGTTTTGCTAAACAACTTTCAACAGTTTCAGCGGAGTGAGAATAGAAAGGAACAACTAA | 1464-1540 |
| AGGAATTGCGAATAATAATTTTTTCACGTTGAAAATCTCCAAAAAAAAAGGCTCCAAAAGGAGCCTTTAATTGTATC | 1541-1617 |
| GGTTTATCAGCTTGCTTTCGAGGTGAATTTCTTAAACAGCTTGATACCGATAGTTGCGCCGACAATGACAACAACCA | 1618-1694 |
| TCGCCCACGCATAACCGATATATTCGGTCGCTGAGGCTTGCAGGGAGTCAAAGGCCGCTTTTGCGGGGATCCGCTCG | 1695-1771 |
| GAGGCGCGGTCGCGGCGCGGCTGTGGCATGTCGGGGCGTGCCGCTCCCCCGGCGCCGCCCATCGGCCCGCCCATTGG | 1772-1848 |
| CATTCCGCCCATGCCGCCCATCATTCCTGTGGAGCCAGAACTGATCCAGCCTGTGCCACAGCCGACAGGATGGTGAC | 1849-1925 |
| CACCATTTGCCCCATATCACCGTCGGTACTGATCCCGTCGTCAATAAACCGAACCGCTACACCCTGAGCATCAAACT | 1926-2002 |
| CTTTTATCAGTTGGATCATGTCGGCGGTGTCGCGGCCAAGACGGTCGAGCTTCTTCACCAGAATGACATCACCTTCC | 2003-2079 |
| TCCACCTTCATCCTCAGCAAATCCAGCCCTTCCCGATCTGTTGAACTGCCGGATGCCTTGTCGGTAAAGATGCGGTT | 2080-2156 |
| AGCTTTTACCCCTGCATCTTTGAGCGCTGAGGTCTGCCTCGTGAAGAAGGTGTTGCTGACTCATACCAGGCCTGAAT | 2157-2233 |
| CGCCCCATCATCCAGCCAGAAAGTGAGGGAGCCACGGTTGATGAGAGCTTTGTTGTAGGTGGACCAGTTGGTGATTT | 2234-2310 |
| TGAACTTTTGCTTTGCCACGGAACGGTCTGCGTTGTCGGGAAGATGCGTGATCTGATCCTTCAACTCAGCAAAAGTT | 2311-2387 |
| CGATTTATTCAACAAAGCCGCCGTCCCGTCAAGTCAGCGTAATGCTCTGCCAGTGTTACAACCAATTAACCAATTCT | 2388-2464 |
| GATTAGAAAAACTCATCGAGCATCAAATGAAACTGCAATTTATTCATATCAGGATTATCAATACCATATTTTTGAAA | 2465-2541 |
| AAGCCGTTTCTGTAATGAAGGAGAAAACTCACCGAGGCAGTTCCATAGGATGGCAAGATCCTGGTATCGGTCTGCGA | 2542-2618 |
| TTCCGACTCGTCCAACATCAATACAACCTATTAATTTCCCCTCGTCAAAAATAAGGTTATCAAGTGAGAAATCACCA | 2619-2695 |
| TGAGTGACGACTGAATCCGGTGAGAATGGCAAAAGCTTATGCATTTCTTTCCAGACTTGTTCAACAGGCCAGCCATT | 2696-2772 |
| ACGCTCGTCATCAAAATCACTCGCATCAACCAAACCGTTATTCATTCGTGATTGCGCCTGAGCGAGACGAAATACGC | 2773-2849 |
| GATCGCTGTTAAAAGGACAATTACAAACAGGAATCGAATGCAACCGGCGCAGGAACACTGCCAGCGCATCAACAATA | 2850-2926 |
| TTTTCACCTGAATCAGGATATTCTTCTAATACCTGGAATGCTGTTTTCCCGGGGATCGCAGTGGTGAGTAACCATGC | 2927-3003 |
| ATCATCAGGAGTACGGATAAAATGCTTGATGGTCGGAAGAGGCATAAATTCCGTCAGCCAGTTTAGTCTGACCATCT | 3004-3080 |
| CATCTGTAACATCATTGGCAACGCTACCTTTGCCATGTTTCAGAAACAACTCTGGCGCATCGGGCTTCCCATACAAT | 3081-3157 |
| CGATAGATTGTCGCACCTGATTGCCCGACATTATCGCGAGCCCATTTATACCCATATAAATCAGCATCCATGTTGGA | 3158-3234 |
| ATTTAATCGCGGCCTCGAGCAAGACGTTTCCCGTTGAATATGGCTCATAACACCCCTTGTATTACTGTTTATGTAAG | 3235-3311 |
| CAGACAGTTTTATTGTTCATGATGATATATTTTTATCTTGTGCAATGTAACATCAGAGATTTTGAGACACAACGTGG | 3312-3388 |
| CTTTGTTGAATAAATCGAACTTTTGCTGAGTTGAAGGATCAGATCACGCATCTTCCCGACAACGCAGACCGTTCCGT | 3389-3465 |
| GGCAAAGCAAAAGTTCAAAATCACCAACTGGTCCACCTACAACAAAGCTCTCATCAACCGTGGCTCCCTCACTTTCT | 3466-3542 |
| GGCTGGATGATGGGGCGATTCAGGCCTGGTATGAGTCAGCAACACCTTCTTCACGAGGCAGACCTCAGCGCTAGCGG | 3543-3619 |
| AGTGTATACTGGCTTACTATGTTGGCACTGATGAGGGTGTCAGTGAAGTGCTTCATGTGGCAGGAGAAAAAAGGCTG | 3620-3696 |
| CACCGGTGCGTCAGCAGAATATGTGATACAGGATATATTCCGCTTCCTCGCTCACTGACTCGCTACGCTCGGTCGTT | 3697-3773 |
| CGACTGCGGCGAGCGGAAATGGCTTACGAACGGGGCGGAGATTTCCTGGAAGATGCCAGGAAGATACTTAACAGGGA | 3774-3850 |
| AGTGAGAGGGCCGCGGCAAAGCCGTTTTTCCATAGGCTCCGCCCCCCTGACAAGCATCACGAAATCTGACGCTCAAA | 3851-3927 |
| TCAGTGGTGGCGAAACCCGACAGGACTATAAAGATACCAGGCGTTTCCCCCTGGCGGCTCCCTCGTGCGCTCTCCTG | 2928-4004 |
| TTCCTGCCTTTCGGTTTACCGGTGTCATTCCGCTGTTATGGCCGCGTTTGTCTCATTCCACGCCTGACACTCAGTTC | 4005-4081 |
| CGGGTAGGCAGTTCGCTCCAAGCTGGACTGTATGCACGAACCCCCCGTTCAGTCCGACCGCTGCGCCTTATCCGGTA | 4082-4158 |
| ACTATCGTCTTGAGTCCAACCCGGAAAGACATGCAAAAGCACCACTGGCAGCAGCCACTGGTAATTGATTTAGAGGA | 4159-4235 |
| GTTAGTCTTGAAGTCATGCGCCGGTTAAGGCTAAACTGAAAGGACAAGTTTTGGTGACTGCGCTCCTCCAAGCCAGT | 4236-4312 |
| TACCTCGGTTCAAAGAGTTGGTAGCTCAGAGAACCTTCGAAAAACCGCCCTGCAAGGCGGTTTTTTCGTTTTCAGAG | 4313-4389 |
| CAAGAGATTACGCGCAGACCAAAACGATCTCAAGAAGATCATCTTATTAAGGGGTCTGACGCTCAGTGGAACGAAAA | 4390-4466 |
| CTCACGTTAAGGGATTTTGGTCATGAGATTATCAAAAAGGATCTTCACCTAGATCCTTTTAAAAGTGCTCATCATTG | 4467-4543 |
| GAAAACGTTCTTCGGGGCGAAAACTCTCAAGGATCTTACCGCTGTTGAGATCCAGTTCGATGTAACCCACTCGTGCA | 4544-4620 |
| CCCAACTGATCTTCAGCATCTTTTACTTTCACCAGCGTTTCTGGGTGAGCAAAAACAGGAAGGCAAAATGCCGCAAA | 4621-4697 |
| AAAGGGAATAAGGGCGACACGGAAATGTTGAATACTCATACTCTTCCTTTTTCAATATTATTGAAGCATTTATCAGG | 4698-4774 |
| GTTATTGTCTCATGAGCGGATACATATTTGAATGTATTTAGAAAAATAAACAAATAGGGGTTCCGCGCACATTTCCC | 4775-4851 |
| CGAAAAGTGCCACCTGACGTCTAAGAAACCATTATTATCATGACATTAACCTATAAAAATAGGCGTATCACGAGGCC | 4852-4928 |
| CTTTCGTCTTCAAGAATTCCCAGGCATCAAATAAAACGAAAGGCTCAGTCGAAAGACTGGGCCTTTCGTTTTATCTG | 4929-5005 |
| TTGTTTGTCGGTGAACGCTCTCCTGAGTAGGACAAATCCGCCGGGAGCGGATTTGAACGTTGCGAAGCAACGGCCCG | 5006-5082 |
| GAGGGTGGCGGGCAGGACGCCCGCCATAAACTGCCAGGGAATTCCCATCGAGCCGAGAACGTTATCGAAGTTGGTCA | 5083-5159 |
| TGTGTAATCCCCTCGTTTGAACTTTGGATTAAGCGTAGATACACCCTTGGACAAGCCAGTTGGATTCGGAGACAAGC | 5160-5236 |
| AAATTCAGCCTTAAAAAGGGCGAGGCCCTGCGGTGGTGGAACACCGCAGGGCCTCTAACCGCTCGACGCGCTGCACC | 5237-5313 |
| AACCAGCCCGCGAACGGCTGGCAGCCAGCGTAAGGCGCGGCTCATCGGGCGGCGTTCGCCACGATGTCCTGCACTTC | 5314-5390 |
| GAGCCAAGCCTCGAACACCTGCTGGTGTGCACGACTCACCCGGTTGTTGACACCGCGCGCGGCCGTGCGGGCTCGGT | 5391-5467 |
| GGGGCGGCTCTGTCGCCCTTGCCAGCGTGAGTAGCGCGTACCTCACCTCGCCCAACAGGTCGCACACAGCCGATTCG | 5468-5544 |
| TACGCCATAAAGCCAGGTGAGCCCACCAGCTCCGTAAGTTCGGGCGCTGTGTGGCTCGTACCCGCGCATTCAGGCGG | 5545-5621 |
| CAGGGGGTCTAACGGGTCTAAGGCGGCGTGTACGCGGCCACAGCGGCTCTCAGCGGCCCGGAAACGTCCTCGAAACG | 5622-5698 |
| ACGCATGTGTTCCTCCTGGTTGGTACAGGTGGTTGGGGGTGCTCGGCTGTCGCGGTTGTTCCACCACCAGGGCTCGA | 5699-5775 |
| CGGGAGAGCGGGGGAGTGTGCAGTTGTGGGGTGGCCCCTCAGCGAAATATCTGACTTGGAGCTCGTGTCGGACCATA | 5776-5852 |
| CACCGGTGATTAATCGTGGTCTACTACCAAGCGTGAGCCACGTCGCCGACGAATTTGAGCAGCTCTGGCTGCCGTAC | 5853-5929 |
| TGGCCGCTGGCAAGCGACGATCTGCTCGAGGGGATCTACCGCCAAAGCCGCGCGTCGGCCCTAGGCCGCCGGTACAT | 5930-6006 |
| CGAGGCGAACCCAACAGCGCTGGCAAACCTGCTGGTCGTGGACGTAGACCATCCAGACGCAGCGCTCCGAGCGCTCA | 6007-6083 |
| GCGCCCGGGGGTCCCATCCGCTGCCCAACGCGATCGTGGGCAATCGCGCCAACGGCCACGCACACGCAGTGTGGGCA | 6084-6160 |
| CTCAACGCCCCTGTTCCACGCACCGAATACGCGCGGCGTAAGCCGCTCGCATACATGGCGGCGTGCGCCGAAGGCCT | 6161-6237 |
| TCGGCGGCCGTCGACGGCGACCGCAGTTACTCAGGCCTCATGACCAAAAACCCCGGCCACATCGCCTGGGAAACGGA | 6238-6314 |
| ATGGCTCCACTCAGATCTCTACACACTCAGCCACATCGAGGCCGAGCTCGGCGCGAACATGCCACCGCCGCGCTGGC | 6315-6391 |
| GTCAGCAGACCACGTACAAAGCGGCTCCGACGCCGCTAGGGCGGAATTGCGCACTGTTCGATTCCGTCAGGTTGTGG | 6392-6468 |
| GCCTATCGTCCCGCCCTCATGCGGATCTACCTGCCGACCCGGAACGTGGACGGACTCGGCCGCGCGATCTATGCCGA | 6469-6545 |
| GTGCCACGCGCGAAACGCCGAATTCCCGTGCAACGACGTGTGTCCCGGACCGCTACCGGACAGCGAGGTCCGCGCCA | 6546-6622 |
| TCGCCAACAGCATTTGGCGTTGGATCACAACCAAGTCGCGCATTTGGGCGGACGGGATCGTGGTCTACGAGGCCACA | 6623-6699 |
| CTCAGTGCGCGCCAGTCGGCCATCTCGCGGAAGGGCGCAGCAGCGCGCACGGCGGCGAGCACAGTTGCGCGGCGCGC | 6700-6776 |
| AAAGTCCGCGTCAGCCATGGAGGCATTGCTATGAGCGACGGCTACAGCGACGGCTACAGCGACGGCTACAACCGGCA | 6777-6853 |
| GCCGACTGTCCGCAAAAAGCCGTGACGCGCCGAAGGCGCTCGAATCACCGGACTATCCGAACGCCACGTCGTCCGGC | 6854-6930 |
| TCGTGGCGCAGGAACGCAGCGAGTGGCTCGCCGAGCAGGCTGCACGCGCGCGAAGCATCCGCGCCTATCACGACGAC | 6931-7007 |
| GAGGGCCACTCTTGGCCGCAAACGGCCAAACATTTCGGGCTGCATCTGGACACCGTTAAGCGACTCGGCTATCGGGC | 7008-7084 |
| GAGGAAAGAGCGTGCGGCAGAACAGGAAGCGGCTCAAAAGGCCCACAACGAAGCCGACAATCCACCGCTGTTCTAAC | 7085-7161 |
| GCAATTGGGGACGGGTGTCGCGGGGGTTCCGTGGGGGGTTCCGTTGCAACGGGTCGGACAGGTAAAAGTCCTGGTAG | 7162-7238 |
| ACGCTAGTTTTCTGGTTTGGGCCATGCCTGTCTCGTTGCGTGTTTCGTTGCGCCGTTTTGAATACCAGCCAGACGAG | 7239-7315 |
| ACGGGGTTCTACGAATCTTGGTCGATACCAAGCCATTTCCGCTGAATATCGGGGAGCTCACCGCCAGAATCGGTGGT | 7316-7392 |
| TGTGGTGATGTACGTGGCGAACTCCGTTGTAGTGCCTGTGGTGGCATCCGTGGCCACTCTCGTTGCACGGTTCGTTG | 7393-7469 |
| TGCCGTTACAGGCCCCGTTGACAGCTCACCGAACGTAGTTAAAACATGCTGGTCAAACTAGGTTTACCAACGATACG | 7470-7546 |
| AGTCAGCTCATCTAGGGCCAGTTCTAGGCGTTGTTCGTTGCGCGGTTCGTTGCGCATGTTTCGTGTGGTTGCTAGAT | 7547-7623 |
| GGCTCCGCAACCACACGCTTCGAGGTTGAGTGCTTCCAGCACGGGCGCGATCCAGAAGAACTTCGTCGTGCGACTGT | 7624-7700 |
| CCTCGTTGGGATCTAGCCCGCCTAATGAGCGGGCTTTTTTTT | 7701-7742 |

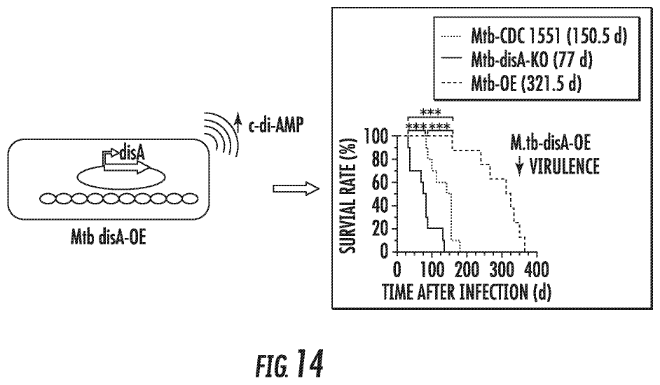

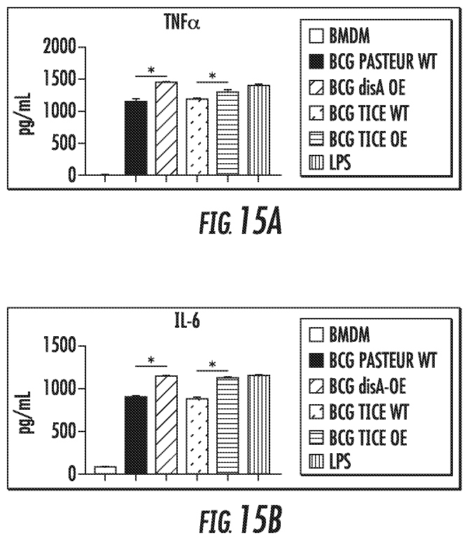

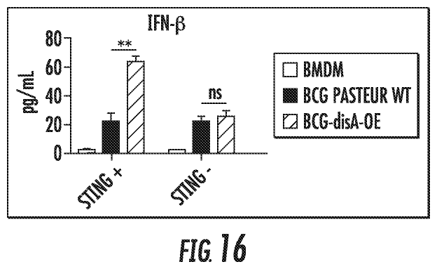

Mycobacteria overexpressing disA are attenuated for virulence. As shown in FIG. 14, when mice are infected with 3.5 log₁₀ units by the aerosol route of either M tuberculosis harboring the pSD5B Pₕₛₚ₆₀::disA plasmid (M.tb-disA-OE or Mtb-OE) or wild type M. tuberculosis (Mtb-CDC1551), there are profound differences in the median time to death (MTD) of the animals. As can be seen, wild type M. tuberculosis (Mtb-CDC1551) gave an MTD of 150.5 days, while M. tuberculosis harboring the pSD5B Pₕₛₚ₆₀::disA plasmid (M.tb-disA-OE or Mtb-OE) was a significantly weaker pathogen giving an MTD of 321.5 days. A similar reduction in the pathogenicity is to be expected with BCG-disA-OE compared with BCG-WT. Hence, it is likely that should BCG-disA-OE be used as a cancer immunotherapy, one would anticipate reduced rates of bloodstream dissemination, reduced dysuria, reduced urgency and reduced malaise compared with BCG-WT.

Addition of CDN Cyclase Genes to rBCG other than disA

Overexpression of the PAMP immunomodulator, 3′-5′ c-di-GMP by overexpressing the GGDEF domain of protein BCG_RS07340. 3′-5′ c-di-GMP is a strong inducer of the STING-TBK1-IRF3 axis. It is produced by mycobacteria including BCG by the GGDEF domain of protein BCG_RS07340 (previously BCG_1416c) and by the M. tuberculosis Rv1354c gene. The BCG_RS07340 protein (100% identical to the M. tuberculosis Rv1354c protein) encodes a bifunctional diguanylate cyclase/diguanylate phosphodiesterase. Hence the portion that functions as a diguanylate cyclase is an endogenous CDN-producing enzyme in BCG. The full-length BCG_RS07340 polypeptide is 623 amino acids in length, and its domain structure is: N-terminus-GAF-GGDEF-EAL-C-terminus as shown in FIG. 11. The GAF domain (approximately amino acids 1-190) is a regulatory domain which influences the activity of the other domains. The GGDEF domain (approximately amino acids 190-350) is a diguanylate cyclase catalyzing the reaction 2 GTP→c-di-GMP+2 pyrophosphates. The EAL domain (approximately amino acids 350-623) is a diguanylate phosphodiesterase catalyzing the reaction c-di-GMP→2 GMP. By genetically removing the DNA sequences that encode the C-terminal EAL domain, it is possible to use the DNA encoding the GGDEF domain to generate a recombinant BCG that will overexpress diguanulate cyclase activity. This may be accomplished by also deleting the DNA encoding the regulatory-sensor GAF domain and/or the use of mutations in the DNA encoding the GAF domain to relieve any cyclase inhibitory activity it may possess. Such techniques to generate constitutively active recombinant forms of the BCG_RS07340 protein will produce high levels of c-di-GMP in recombinant BCG.

SEQ ID NO: 4

Bifunctional diguanylate cyclase/phosphodiesterase BCG_RS07340 from BCG and other related mycobacteria, amino acid sequence (623 amino acids; BCG protein BCG_RS07340; NCBI Reference Sequence: NC_008769.1; Protein ID WP 003898837.1; old locus tag BCG_1416c). The identical sequence is present in other strains of BCG, e.g., Mycobacterium tuberculosis as protein Rv1354c or MT1397, and in Mycobacterium bovis as protein Mb1389c. The EAL domain is from amino acid 354 to 623 and is underlined.

MCNDTATPQLEELVTTVANQLMTVDAATSAEVSQRVLAYLVEQLGVDVSF
LRHNDRDRRATRLVAEWPPRLNIPDPDPLRLIYFADADPVFALCEHAKEP
LVFRPEPATEDYQRLIEEARGVPVTSAAAVPLVSGEITTGLLGFIKFGDR
KWHEAELNALMTIATLFAQVQARVAAEARLRYLADHDDLTGLHNRRALLQ
HLDQRLAPGQPGPVAALFLDLDRLKAINDYLGHAAGDQFIHVFAQRIGDA
LVGESLIARLGGDEFVLIPASPMSADAAQPLAERLRDQLKDHVAIGGEVL
TRTVSIGVASGTPGQHTPSDLLRRADQAALAAKHAGGDSVAIFTADMSVS
GELRNDIELHLRRGIESDALRLVYLPEVDLRTGDIVGTEALVRWQHPTRG
LLAPGCFIPVAESINLAGELDRWVLRRACNEFSEWQSAGLGHDALLRINV
SAGQLVTGGFVDFVADTIGQHGLDASSVCLEITENVVVQDLHTARATLAR
LKEVGVHIAIDDFGTGYSAISLLQTLPIDTLKIDKTFVRQLGTNTSDLVI
VRGIMTLAEGFQLDVVAEGVETEAAARILLDQRCYRAQGFLFSRPVPGEA
MRHMLSARRLPPTCIPATDPALS

SEQ ID NO: 5

Bifunctional diguanylate cyclase/phosphodiesterase BCG_RS07340 from BCG and other related mycobacteria, DNA sequence (1872 nucleotides [623 codons+1 stop codons]; encodes BCG protein BCG_RS07340; NCBI Reference Sequence: NC_008769.1; Protein ID WP 003898837.1; old locus tag BCG_1416c; DNA from NC_008769.1:c1548390-1546519 Mycobacterium bovis BCG Pasteur 1173P2). The identical sequence is present in other strains of BCG, e.g., Mycobacterium tuberculosis as protein Rv1354c or MT1397, and in Mycobacterium bovis as protein Mb1389c. EAL domain is encoded from nucleotide 1060 to 1872 and is underlined.

ATGTGCAACGACACCGCGACGCCGCAGCTTGAGGAGCTCGTCACCACCGT
AGCCAACCAGCTCATGACAGTCGACGCTGCCACGTCAGCCGAAGTCAGTC
AGCGCGTTTTGGCCTATCTAGTGGAACAGCTGGGCGTAGATGTCAGCTTT
TTGCGTCATAACGATCGCGACAGGCGCGCGACGAGGCTGGTGGCCGAATG
GCCACCTCGCCTCAACATACCGGACCCCGATCCGCTCAGGCTGATCTACT
TCGCTGATGCCGACCCGGTGTTTGCGCTATGCGAACACGCCAAAGAGCCT
CTCGTGTTCCGGCCCGAGCCGGCCACCGAGGACTATCAACGCCTCATCGA
AGAAGCCCGCGGGGTTCCGGTAACGTCGGCTGCCGCCGTGCCGCTGGTAT
CTGGCGAGATCACCACTGGACTGCTGGGGTTCATCAAGTTCGGTGATCGG
AAATGGCACGAGGCCGAGCTTAACGCCCTCATGACCATCGCTACACTCTT
CGCCCAGGTGCAGGCTCGCGTCGCCGCCGAGGCGCGGCTTCGCTATCTGG
CCGACCATGACGATCTGACCGGACTGCATAACCGTCGCGCGTTGCTGCAG
CACCTGGACCAAAGACTGGCCCCCGGACAACCTGGCCCGGTCGCGGCGCT
ATTTCTCGACTTGGACCGCCTCAAGGCCATCAACGACTACCTGGGCCACG
CCGCCGGTGACCAGTTCATCCATGTGTTCGCCCAACGGATCGGTGACGCA
CTCGTTGGCGAGAGCCTGATCGCCCGACTCGGCGGCGACGAATTCGTCCT
CATACCCGCATCTCCAATGAGTGCCGATGCCGCTCAACCGCTCGCCGAAC
GTCTTCGCGACCAGCTCAAGGACCACGTCGCTATCGGCGGTGAGGTGCTC
ACCCGCACCGTCAGTATCGGTGTCGCCTCAGGGACTCCCGGACAGCACAC
ACCGTCGGACCTCCTGCGCCGAGCCGACCAAGCCGCTCTGGCAGCCAAAC
ACGCCGGCGGAGATAGCGTCGCGATTTTCACCGCGGACATGTCGGTCAGC
GGCGAACTGCGCAACGATATTGAACTACACCTTCGACGTGGTATCGAATC
CGACGCCCTTCGCCTGGTCTACCTACCCGAGGTCGACCTACGGACCGGCG
ACATTGTCGGGACCGAGGCATTGGTCCGGTGGCAGCACCCCACCCGTGGG
CTGCTGGCACCGGGCTGCTTCATCCCTGTGGCCGAATCCATCAACCTTGC
AGGCGAATTGGATAGATGGGTGCTGCGGAGGGCCTGCAATGAATTCTCCG
AGTGGCAGTCAGCCGGTTTGGGCCACGACGCGCTGCTGCGTATCAACGTC
TCAGCTGGACAGCTGGTGACGGGCGGGTTTGTTGACTTCGTCGCAGACAC
GATCGGCCAGCACGGTCTGGACGCCTCGTCCGTGTGTTTGGAAATCACCG
AAAACGTTGTGGTGCAAGACCTACATACCGCCAGAGCCACCCTGGCTCGA
CTCAAAGAAGTCGGCGTTCACATCGCTATCGACGATTTCGGCACCGGCTA
TAGCGCCATATCACTGTTGCAGACGCTACCGATCGACACGCTCAAGATCG
ACAAAACATTCGTGCGGCAACTCGGAACCAACACTAGCGATCTGGTCATT
GTGCGCGGCATCATGACACTCGCCGAAGGCTTCCAACTCGATGTAGTAGC
CGAAGGCGTCGAGACCGAGGCTGCCGCCAGAATTCTATTGGATCAGCGCT
GTTACCGTGCGCAAGGCTTCTTGTTCTCCCGGCCTGTCCCCGGGGAGGCC
ATGCGGCACATGTTGTCCGCACGACGACTACCGCCGACCTGCATACCTGC
AACTGACCCGGCGTTATCTTGA

SEQ ID NO: 6

Modified bifunctional diguanylate cyclase/phosphodiesterase from BCG and other related mycobacteria, with its EAL domain deleted so that it acts as a monofunctional diguanylate cyclase, amino acid sequence (353 amino acids; a fragment of BCG protein BCG_RS07340; NCBI Reference Sequence: NC_008769.1; Protein ID WP 003898837.1; old locus tag BCG_1416c). The identical sequence fragment is present in other strains of BCG, e.g., Mycobacterium tuberculosis as protein Rv1354c or MT1397, and in Mycobacterium bovis as protein Mb1389c.

MCNDTATPQLEELVTTVANQLMTVDAATSAEVSQRVLAYLVEQL
GVDVSFLRHNDRDRRATRLVAEWPPRLNIPDPDPLRLIYFADADPVFALC
EHAKEPLVFRPEPATEDYQRLIEEARGVPVTSAAAVPLVSGEITTGLLGF
IKFGDRKWHEAELNALMTIATLFAQVQARVAAEARLRYLADHDDLTGLHN
RRALLQHLDQRLAPGQPGPVAALFLDLDRLKAINDYLGHAAGDQFIHVFA
QRIGDALVGESLIARLGGDEFVLIPASPMSADAAQPLAERLRDQLKDHVA
IGGEVLTRTVSIGVASGTPGQHTPSDLLRRADQAALAAKHAGGDSVAIFT
ADMSVSGEL

SEQ ID NO: 7

Modified, bifunctional diguanylate cyclase/phosphodiesterase from BCG and other related mycobacteria, with sequences encoding its EAL domain deleted so that it encodes a monofunctional diguanylate cyclase, DNA sequence (1059 nucleotides [353 codons+0 stop codons]; encodes a fragment of BCG protein BCG_RS07340; NCBI Reference Sequence: NC_008769.1; Protein ID WP 003898837.1; old locus tag BCG_1416c; DNA from NC_008769.1:c1548390-1546519 Mycobacterium bovis BCG Pasteur 1173P2). The identical sequence is present in other strains of BCG, e.g., Mycobacterium tuberculosis as a fragment of gene Rv1354c or MT1397, and in Mycobacterium bovis as a fragment of gene Mb1389c.

ATGTGCAACGACACCGCGACGCCGCAGCTTGAGGAGCTCGTCACCACCGT
AGCCAACCAGCTCATGACAGTCGACGCTGCCACGTCAGCCGAAGTCAGTC
AGCGCGTTTTGGCCTATCTAGTGGAACAGCTGGGCGTAGATGTCAGCTTT
TTGCGTCATAACGATCGCGACAGGCGCGCGACGAGGCTGGTGGCCGAATG
GCCACCTCGCCTCAACATACCGGACCCCGATCCGCTCAGGCTGATCTACT
TCGCTGATGCCGACCCGGTGTTTGCGCTATGCGAACACGCCAAAGAGCCT
CTCGTGTTCCGGCCCGAGCCGGCCACCGAGGACTATCAACGCCTCATCGA
AGAAGCCCGCGGGGTTCCGGTAACGTCGGCTGCCGCCGTGCCGCTGGTAT
CTGGCGAGATCACCACTGGACTGCTGGGGTTCATCAAGTTCGGTGATCGG
AAATGGCACGAGGCCGAGCTTAACGCCCTCATGACCATCGCTACACTCTT
CGCCCAGGTGCAGGCTCGCGTCGCCGCCGAGGCGCGGCTTCGCTATCTGG
CCGACCATGACGATCTGACCGGACTGCATAACCGTCGCGCGTTGCTGCAG
CACCTGGACCAAAGACTGGCCCCCGGACAACCTGGCCCGGTCGCGGCGCT
ATTTCTCGACTTGGACCGCCTCAAGGCCATCAACGACTACCTGGGCCACG
CCGCCGGTGACCAGTTCATCCATGTGTTCGCCCAACGGATCGGTGACGCA
CTCGTTGGCGAGAGCCTGATCGCCCGACTCGGCGGCGACGAATTCGTCCT
CATACCCGCATCTCCAATGAGTGCCGATGCCGCTCAACCGCTCGCCGAAC
GTCTTCGCGACCAGCTCAAGGACCACGTCGCTATCGGCGGTGAGGTGCTC
ACCCGCACCGTCAGTATCGGTGTCGCCTCAGGGACTCCCGGACAGCACAC
ACCGTCGGACCTCCTGCGCCGAGCCGACCAAGCCGCTCTGGCAGCCAAAC
ACGCCGGCGGAGATAGCGTCGCGATTTTCACCGCGGACATGTCGGTCAGC
GGCGAACTG

Overexpression of the PAMP immunomodulator, 2′-5′c-GAMP synthase: Q9KVG7 (Swiss-Prot). 2′-5′ c-GAMP is a strong inducer of the STING-TBK1-IRF3 axis. The Vibrio cholerae Q9KVG7 protein (436 amino acids) encoded by the dncV gene is a known 2′-5′c-GAMP synthase. It is possible to generate a recombinant dncV gene which is codon-optimized for BCG. The codon-optimized structural gene may be overexpressed in BCG by fusion to a strong promoter (such as Phsp60) or a conditionally active strong promoter such as PTET-off. Such techniques to generate a constitutively active recombinant forms of the Q9KVG7 protein will produce high levels of 2′-5′c-GAMP in recombinant BCG.

SEQ ID No: 8

Cyclic GMP-AMP synthase, DncV, from Vibrio cholerae, amino acid sequence (436 amino acids; UniProtKB/Swiss-Prot Protein ID Q9KVG7.1).

MRMTWNFHQYYTNRNDGLMGKLVLTDEEKNNLKALRKIIRLRTRDVFEEA
KGIAKAVKKSALTFEIIQEKVSTTQIKHLSDSEQREVAKLIYEMDDDARD
EFLGLTPRFWTQGSFQYDTLNRPFQPGQEMDIDDGTYMPMPIFESEPKIG
HSLLILLVDASLKSLVAENHGWKFEAKQTCGRIKIEAEKTHIDVPMYAIP
KDEFQKKQIALEANRSFVKGAIFESYVADSITDDSETYELDSENVNLALR
EGDRKWINSDPKIVEDWFNDSCIRIGKHLRKVCRFMKAWRDAQWDVGGPS
SISLMAATVNILDSVAHDASDLGETMKIIAKHLPSEFARGVESPDSTDEK
PLFPPSYKHGPREMDIMSKLERLPEILSSAESADSKSEALKKINMAFGNR
VTNSELIVLAKALPAFAQEPSSASKPEKISSTMVSG

SEQ ID No: 9

Cyclic GMP-AMP synthase, DncV, from Vibrio cholerae, DNA sequence (1311 nucleotides [436 codons+1 stop codon]; encodes UniProtKB/Swiss-Prot Protein ID Q9KVG7.1; NCBI Reference Sequence: NC_002505.1: Vibrio cholerae 01 biovar El Tor str. N16961 chromosome I, complete sequence, and nucleotides 180419-181729)

GTGAGAATGACTTGGAACTTTCACCAGTACTACACAAACCGAAATGATGG
CTTGATGGGCAAGCTAGTTCTTACAGACGAGGAGAAGAACAATCTAAAGG
CATTGCGTAAGATCATCCGCTTAAGAACACGAGATGTATTTGAAGAAGCT
AAGGGTATTGCCAAGGCTGTGAAAAAAAGTGCTCTTACGTTTGAAATTAT
TCAGGAAAAGGTGTCAACGACCCAAATTAAGCACCTTTCTGACAGCGAAC
AACGAGAAGTGGCTAAGCTTATTTACGAGATGGATGATGATGCTCGTGAT
GAGTTTTTGGGATTGACACCTCGCTTTTGGACTCAGGGAAGCTTTCAGTA
TGACACGCTGAATCGCCCGTTTCAGCCTGGTCAAGAAATGGATATTGATG
ATGGAACCTATATGCCAATGCCTATTTTTGAGTCAGAGCCTAAGATTGGT
CATTCTTTACTAATTCTTCTTGTTGACGCGTCACTTAAGTCACTTGTAGC
TGAAAATCATGGCTGGAAATTTGAAGCTAAGCAGACTTGTGGGAGGATTA
AGATTGAGGCAGAGAAAACACATATTGATGTACCAATGTATGCAATCCCT
AAAGATGAGTTCCAGAAAAAGCAAATAGCTTTAGAAGCAAATAGATCATT
TGTTAAAGGTGCCATTTTTGAATCATATGTTGCAGATTCAATTACTGACG
ATAGTGAAACTTATGAATTAGATTCAGAAAACGTAAACCTTGCTCTTCGT
GAAGGTGATCGGAAGTGGATCAATAGCGACCCCAAAATAGTTGAAGATTG
GTTCAACGATAGTTGTATACGTATTGGTAAACATCTTCGTAAGGTTTGTC
GCTTTATGAAAGCGTGGAGAGATGCGCAGTGGGATGTTGGAGGTCCGTCA
TCGATTAGTCTTATGGCTGCAACGGTAAATATTCTTGATAGCGTTGCTCA
TGATGCTAGTGATCTCGGAGAAACAATGAAGATAATTGCTAAGCATTTAC
CTAGTGAGTTTGCTAGGGGAGTAGAGAGCCCTGACAGTACCGATGAAAAG
CCACTCTTCCCACCCTCTTATAAGCATGGCCCTCGGGAGATGGACATTAT
GAGCAAACTAGAGCGTTTGCCAGAGATTCTGTCATCTGCTGAGTCAGCTG
ACTCTAAGTCAGAGGCCTTGAAAAAGATTAATATGGCGTTTGGGAATCGT
GTTACTAATAGCGAGCTTATTGTTTTGGCAAAGGCTTTACCGGCTTTCGC
TCAAGAACCTAGTTCAGCCTCGAAACCTGAAAAAATCAGCAGCACAATGG
TAAGTGGCTGA

Overexpression of the DAMP immunomodulator, 2′-3′ cGAMP synthase: Q8N884 (Swiss-Prot). 2′-3′ cGAMP is a strong inducer of the STING-TBK1-IRF3 axis. The cGAS protein is produced by the human cGAS gene to yield a 522 amino acid polypeptide which senses cytosolic DNA and functions as a 2′-3′ cGAMP synthase. The synthase or cyclase domain of cGAS becomes activated when cGAS binds to DNA. It is possible to generate a recombinant cGAS gene which contains only the cyclase domain and is hence constitutively active. This recombinant gene can also be codon-optimized for BCG. The codon-optimized structural gene may be overexpressed in BCG by fusion to a strong promoter (such as Phsp60) or a conditionally active strong promoter such as PTET-off. Such techniques to generate a constitutively active recombinant forms of the cGAS protein will produce high levels of 2′-3′c-GAMP in recombinant BCG.

SEQ ID No: 10

Cyclic 2′3′-GMP-AMP synthase, cGAS, from Homo sapiens, amino acid sequence (522 amino acids, UniProtKB/Swiss-Prot Protein ID Q8N884.2).

MQPWHGKAMQRASEAGATAPKASARNARGAPMDPTESPAAPEAALPKAGK
FGPARKSGSRQKKSAPDTQERPPVRATGARAKKAPQRAQDTQPSDATSAP
GAEGLEPPAAREPALSRAGSCRQRGARCSTKPRPPPGPWDVPSPGLPVSA
PILVRRDAAPGASKLRAVLEKLKLSRDDISTAAGMVKGVVDHLLLRLKCD
SAFRGVGLLNTGSYYEHVKISAPNEFDVMFKLEVPRIQLEEYSNTRAYYF
VKFKRNPKENPLSQFLEGEILSASKMLSKFRKIIKEEINDIKDTDVIMKR
KRGGSPAVTLLISEKISVDITLALESKSSWPASTQEGLRIQNWLSAKVRK
QLRLKPFYLVPKHAKEGNGFQEETWRLSFSHIEKEILNNHGKSKTCCENK
EEKCCRKDCLKLMKYLLEQLKERFKDKKHLDKFSSYHVKTAFFHVCTQNP
QDSQWDRKDLGLCFDNCVTYFLQCLRTEKLENYFIPEFNLFSSNLIDKRS
KEFLTKQIEYERNNEFPVFDEF

SEQ ID No: 11

Cyclic 2′3′-GMP-AMP synthase, cGAS, from Homo sapiens, DNA sequence of mRNA with nucleotide T used in place of U (1802 nucleotides; encodes UniProtKB/Swiss-Prot Protein ID Q8N884.2; NCBI Reference Sequence: NM_138441.2. Coding sequence is 1569 nucleotides [522 codons, 1 stop codon], start codon ATG [bold underlined] at nucleotide 140; Stop codon TGA (bold, underlined) at nucleotide 1706]).

AGCCTGGGGTTCCCCTTCGGGTCGCAGACTCTTGTGTGCCCGCCAGTAGT
GCTTGGTTTCCAACAGCTGCTGCTGGCTCTTCCTCTTGCGGCCTTTTCCT
GAAACGGATTCTTCTTTCGGGGAACAGAAAGCGCCAGCCATGCAGCCTTG
GCACGGAAAGGCCATGCAGAGAGCTTCCGAGGCCGGAGCCACTGCCCCCA
AGGCTTCCGCACGGAATGCCAGGGGCGCCCCGATGGATCCCACCGAGTCT
CCGGCTGCCCCCGAGGCCGCCCTGCCTAAGGCGGGAAAGTTCGGCCCCGC
CAGGAAGTCGGGATCCCGGCAGAAAAAGAGCGCCCCGGACACCCAGGAGA
GGCCGCCCGTCCGCGCAACTGGGGCCCGCGCCAAAAAGGCCCCTCAGCGC
GCCCAGGACACGCAGCCGTCTGACGCCACCAGCGCCCCTGGGGCAGAGGG
GCTGGAGCCTCCTGCGGCTCGGGAGCCGGCTCTTTCCAGGGCTGGTTCTT
GCCGCCAGAGGGGCGCGCGCTGCTCCACGAAGCCAAGACCTCCGCCCGGG
CCCTGGGACGTGCCCAGCCCCGGCCTGCCGGTCTCGGCCCCCATTCTCGT
ACGGAGGGATGCGGCGCCTGGGGCCTCGAAGCTCCGGGCGGTTTTGGAGA
AGTTGAAGCTCAGCCGCGATGATATCTCCACGGCGGCGGGGATGGTGAAA
GGGGTTGTGGACCACCTGCTGCTCAGACTGAAGTGCGACTCCGCGTTCAG
AGGCGTCGGGCTGCTGAACACCGGGAGCTACTATGAGCACGTGAAGATTT
CTGCACCTAATGAATTTGATGTCATGTTTAAACTGGAAGTCCCCAGAATT
CAACTAGAAGAATATTCCAACACTCGTGCATATTACTTTGTGAAATTTAA
AAGAAATCCGAAAGAAAATCCTCTGAGTCAGTTTTTAGAAGGTGAAATAT
TATCAGCTTCTAAGATGCTGTCAAAGTTTAGGAAAATCATTAAGGAAGAA
ATTAACGACATTAAAGATACAGATGTCATCATGAAGAGGAAAAGAGGAGG
GAGCCCTGCTGTAACACTTCTTATTAGTGAAAAAATATCTGTGGATATAA
CCCTGGCTTTGGAATCAAAAAGTAGCTGGCCTGCTAGCACCCAAGAAGGC
CTGCGCATTCAAAACTGGCTTTCAGCAAAAGTTAGGAAGCAACTACGACT
AAAGCCATTTTACCTTGTACCCAAGCATGCAAAGGAAGGAAATGGTTTCC
AAGAAGAAACATGGCGGCTATCCTTCTCTCACATCGAAAAGGAAATTTTG
AACAATCATGGAAAATCTAAAACGTGCTGTGAAAACAAAGAAGAGAAATG
TTGCAGGAAAGATTGTTTAAAACTAATGAAATACCTTTTAGAACAGCTGA
AAGAAAGGTTTAAAGACAAAAAACATCTGGATAAATTCTCTTCTTATCAT
GTGAAAACTGCCTTCTTTCACGTATGTACCCAGAACCCTCAAGACAGTCA
GTGGGACCGCAAAGACCTGGGCCTCTGCTTTGATAACTGCGTGACATACT
TTCTTCAGTGCCTCAGGACAGAAAAACTTGAGAATTATTTTATTCCTGAA
TTCAATCTATTCTCTAGCAACTTAATTGACAAAAGAAGTAAGGAATTTCT
GACAAAGCAAATTGAATATGAAAGAAACAATGAGTTTCCAGTTTTTGATG
AATTTTGAGATTGTATTTTTAGAAAGATCTAAGAACTAGAGTCACCCTAA
ATCCTGGAGAATACAAGAAAAATTTGAAAAGGGGCCAGACGCTGTGGCTC
AC

SEQ ID NO: 12

Cyclic 2′3′-GMP-AMP synthase, cGAS, from Homo sapiens with mycobacterial codon optimization, DNA sequence. (1569 nucleotides [522 codons, 1 stop codon]; encodes UniProtKB/Swiss-Prot Protein ID Q8N884.2).

ATGCAACCATGGCACGGGAAAGCCATGCAGCGTGCGAGCGAAGCCGGGGC
GACGGCCCCCAAGGCGTCGGCGCGTAACGCGCGGGGTGCGCCCATGGACC
CGACGGAGTCCCCCGCGGCGCCGGAGGCGGCCCTGCCGAAAGCGGGTAAG
TTCGGTCCAGCGCGGAAAAGCGGGAGCCGCCAAAAGAAGTCCGCGCCCGA
CACCCAGGAGCGTCCCCCGGTCCGGGCCACCGGCGCGCGTGCCAAAAAAG
CCCCGCAACGGGCGCAAGATACGCAGCCAAGCGATGCGACCTCCGCCCCC
GGGGCGGAGGGTCTGGAGCCCCCGGCCGCCCGGGAGCCAGCGCTCTCGCG
CGCGGGTTCCTGCCGTCAGCGGGGCGCGCGGTGTTCCACGAAACCCCGTC
CCCCACCAGGTCCCTGGGACGTGCCGTCGCCGGGTTTGCCGGTGAGCGCG
CCAATCCTGGTCCGGCGCGACGCGGCCCCGGGGGCGTCGAAATTGCGTGC
GGTGCTCGAGAAATTGAAGTTGTCGCGCGACGACATCTCCACGGCCGCGG
GTATGGTCAAGGGCGTGGTCGATCATTTGTTGTTGCGGCTCAAGTGTGAT
TCGGCGTTCCGCGGGGTGGGCTTGCTGAACACGGGGTCCTACTATGAGCA
TGTCAAAATCAGCGCCCCCAACGAATTTGACGTGATGTTTAAGCTGGAAG
TGCCACGTATCCAATTGGAAGAGTATTCCAATACCCGTGCGTATTATTTC
GTCAAATTTAAGCGCAATCCGAAGGAAAATCCACTCAGCCAATTCTTGGA
GGGCGAAATTCTGTCGGCCTCGAAAATGCTCTCCAAATTTCGTAAGATTA
TCAAGGAGGAGATCAACGACATTAAGGACACGGATGTGATCATGAAACGT
AAACGTGGCGGTTCCCCCGCGGTGACGCTCCTCATTTCGGAAAAAATTTC
GGTGGACATTACCCTGGCGTTGGAATCGAAGTCCAGCTGGCCGGCGTCGA
CCCAGGAGGGCCTGCGGATTCAAAACTGGTTGAGCGCCAAAGTGCGGAAG
CAGCTGCGTCTCAAACCCTTTTATTTGGTCCCGAAACATGCCAAAGAGGG
TAACGGTTTTCAAGAGGAAACCTGGCGTTTGAGCTTCTCCCACATTGAGA
AGGAGATTTTGAACAACCATGGTAAGTCCAAAACGTGCTGCGAGAATAAG
GAAGAAAAATGTTGTCGCAAAGATTGTCTCAAATTGATGAAATATTTGCT
GGAACAACTCAAAGAGCGTTTTAAGGACAAGAAGCATCTCGACAAGTTCT
CCTCGTATCACGTCAAGACCGCCTTCTTTCATGTCTGTACGCAGAACCCG
CAAGATAGCCAGTGGGATCGCAAGGACTTGGGGTTGTGTTTTGACAATTG
CGTCACCTATTTCTTGCAATGTTTGCGGACCGAGAAATTGGAGAACTACT
TTATTCCAGAATTCAACTTGTTTTCCTCGAATCTGATTGACAAACGCTCC
AAAGAGTTTCTGACGAAGCAGATTGAATACGAGCGTAACAATGAGTTTCC
GGTCTTTGACGAGTTTTGA

SEQ ID NO: 13

Plasmid pMH94H-Pₕₛₚ₆₀::disA::hcGASco::mCherry which is an E. coli-mycobacterial shuttle plasmid that overexpresses the BCG disA gene, the human cGAS gene (with mycobacterial codon optimization), and mCherry from the Pₕₛₚ₆₀ promoter, DNA sequence. When introduced into BCG, M. tuberculosis, M. bovis or highly related strains, this plasmid integrates as a single copy in the mycobacterial chromosome (10842 nucleotides; promoter Pₕₛₚ₆₀ DNA comprised of a portion of the M. leprae hsp65 gene nucleotides 901 to 1068 is underlined; disA coding sequence are from nucleotides 1069 to 2145; human cGAS with mycobacterial codon optimization sequences are from nucleotides 2158 to 3726; ATG start codons and TAA or TGA stop codons are shown in boldface, underline).

| | |
|---|---|
| TCGCGCGTTTCGGTGATGACGGTGAAAACCTCTGACACATGCAGCTCCCGGAGACGGTCACAGCTTGTCTGTAAGCGGATGCC | 1-83 |
| GGGAGCAGACAAGCCCGTCAGGGCGCGTCAGCGGGTGTTGGCGGGTGTCGGGGCTGGCTTAACTATGCGGCATCAGAGCAGAT | 84-166 |
| TGTACTGAGAGTGCACCAAAATTGTAAACGTTAATATTTTGTTAAAATTCGCGTTAAATTTTTGTTAAATCAGCTCATTTTTT | 167-249 |
| AACCAATAGGCCGAAATCGGCAAAATCCCTTATAAATCAAAAGAATAGCCCGAGATAGGGTTGAGTGTTGTTCCAGTTTGGA | 250-331 |
| ACAAGAGTCCACTATTAAAGAACGTGGACTCCAACGTCAAAGGGCGAAAAACCGTCTATCAGGGCGATGGCCCACTACGTGAA | 332-414 |
| CCATCACCCAAATCAAGTTTTTTGGGGTCGAGGTGCCGTAAAGCACTAAATCGGAACCCTAAAGGGAGCCCCCGATTTAGAGC | 415-497 |
| TTGACGGGGAAAGCCGGCGAACGTGGCGAGAAAGGAAGGGAAGAAAGCGAAAGGAGCGGGCGCTAGGGCGCTGGCAAGTGTAG | 498-580 |
| CGGTCACGCTGCGCGTAACCACCACACCCGCCGCGCTTAATGCGCCGCTACAGGGCGCGTACTATGGTTGCTTTGACGTGCGG | 581-663 |
| TGTGAAATACCGCACAGATGCGTAAGGAGAAAATACCGCATCAGGCGCCATTCGCCATTCAGGCTGCGCAACTGTTGGGAAGG | 664-746 |
| GCGATCGGTGCGGGCCTCTTCGCTATTACGCCAGCTGGCGAAAGGGGGATGTGCTGCAAGGCGATTAAGTTGGGTAACGCCAG | 747-829 |
| GGTTTTCCCAGTCACGACGTTGTAAAACGACGGCCAGTGAATTCGAGCTCGGTACCCGGGGATCCTCTAGAAATTCCGGAATT | 830-912 |
| GCACTCGCCTTAGGGGAGTGCTAAAAATGATCCTGGCACTCGCGATCAGCGAGTGCCAGGTCGGGACGGTGAGACCCAGCCAG | 913-995 |
| CAAGCTGTGGTCGTCCGTCGCGGGCACTGCACCCGGCCAGCGTAAGTAATGGGGGTTGTCGGCACCCGGTGACATGCACGCTG | 996-1078 |
| TGACTCGTCCGACCCTGCGTGAGGCTGTCGCCCGCCTAGCCCCGGGCACTGGGCTGCGGGACGGCCTGGAGCGTATCCTGCGC | 1079-1161 |
| GGCCGCACTGGTGCCCTGATCGTGCTGGGCCATGACGAGAATGTCGAGGCCATCTGCGATGGTGGCTTCTCCCTCGATGTCCG | 1162-1244 |
| CTATGCAGCAACCCGGCTACGCGAGCTGTGCAAGATGGACGGCGCCGTGGTGCTGTCCACCGACGGCAGCCGCATCGTGCGGG | 1245-1327 |
| CCAACGTGCAACTGGTACCGGATCCGTCGATCCCCACCGACGAATCGGGGACCCGGCACCGCTCGGCCGAGCGGGCCGCGATC | 1228-1410 |
| CAGACCGGTTACCCGGTGATCTCAGTGAGCCACTCGATGAACATCGTGACCGTCTACGTCCGCGGGGAACGTCACGTATTGAC | 1411-1493 |
| CGACTCGGCAACCATCCTGTCGCGGGCCAACCAGGCCATCGCAACCCTGGAGCGGTACAAAACCAGGCTCGACGAGGTCAGCC | 1494-1576 |
| GGCAACTGTCCAGGGCAGAAATCGAGGACTTCGTCACGCTGCGCGATGTGATGACGGTGGTGCAACGCCTCGAGCTGGTCCGG | 1577-1659 |
| CGAATCGGGCTGGTGATCGACTACGACGTGGTCGAACTCGGCACTGATGGTCGTCAGCTGCGGCTGCAGCTCGACGAGTTGCT | 1660-1742 |
| CGGCGGCAACGACACCGCCCGGGAATTGATCGTGCGCGATTACCACGCCAACCCGGAACCACCGTCCACGGGGCAAATCAATG | 1743-1825 |
| CCACCCTGGACGAACTGGACGCCCTGTCGGACGGCGACCTCCTCGATTTCACCGCGCTGGCAAAGGTTTTCGGATATCCGACG | 1826-1908 |
| ACCACGGAAGCGCAGGATTCGACGCTGAGCCCGCGTGGCTACCGCGCGATGGCCGGTATCCCCCGGCTCCAGTTCGCCCATGC | 1909-1991 |
| CGACCTGCTGGTCCGGGCGTTCGGAACGTTGCAGGGTCTGCTGGCGGCCAGCGCCGGCGATCTGCAATCAGTGGACGGCATCG | 1992-2074 |
| GCGCCATGTGGGCCCGTCATGTGCGCGAGGGGTTGTCACAGCTGGCGGAATCGACCATCAGCGATCAATAAGAGCACATCGAT | 2075-2157 |
| ATGCAACCATGGCACGGGAAAGCCATGCAGCGTGCGAGCGAAGCCGGGGCGACGGCCCCCAAGGCGTCGGCGCGTAACGCGCG | 2158-2240 |
| GGGTGCGCCCATGGACCCGACGGAGTCCCCCGCGGCGCCGGAGGCGGCCCTGCCGAAAGCGGGTAAGTTCGGTCCAGCGCGGA | 2241-2323 |
| AAAGCGGGAGCCGCCAAAAGAAGTCCGCGCCCGACACCCAGGAGCGTCCCCCGGTCCGGGCCACCGGCGCGCGTGCCAAAAAA | 2324-2406 |
| GCCCCGCAACGGGCGCAAGATACGCAGCCAAGCGATGCGACCTCCGCCCCCGGGGCGGAGGGTCTGGAGCCCCCGGCCGCCCG | 2407-2489 |
| GGAGCCAGCGCTCTCGCGCGCGGGTTCCTGCCGTCAGCGGGGCGCGCGGTGTTCCACGAAACCCCGTCCCCCACCAGGTCCCT | 2490-2572 |
| GGGACGTGCCGTCGCCGGGTTTGCCGGTGAGCGCGCCAATCCTGGTCCGGCGCGACGCGGCCCCGGGGGCGTCGAAATTGCGT | 2573-2655 |
| GCGGTGCTCGAGAAATTGAAGTTGTCGCGCGACGACATCTCCACGGCCGCGGGTATGGTCAAGGGCGTGGTCGATCATTTGTT | 2656-2738 |
| GTTGCGGCTCAAGTGTGATTCGGCGTTCCGCGGGGTGGGCTTGCTGAACACGGGGTCCTACTATGAGCATGTCAAAATCAGCG | 2739-2821 |
| CCCCCAACGAATTTGACGTGATGTTTAAGCTGGAAGTGCCACGTATCCAATTGGAAGAGTATTCCAATACCCGTGCGTATTAT | 2822-2904 |
| TTCGTCAAATTTAAGCGCAATCCGAAGGAAAATCCACTCAGCCAATTCTTGGAGGGCGAAATTCTGTCGGCCTCGAAAATGCT | 2905-2987 |
| CTCCAAATTTCGTAAGATTATCAAGGAGGAGATCAACGACATTAAGGACACGGATGTGATCATGAAACGTAAACGTGGCGGTT | 2988-3070 |
| CCCCCGCGGTGACGCTCCTCATTTCGGAAAAAATTTCGGTGGACATTACCCTGGCGTTGGAATCGAAGTCCAGCTGGCCGGCG | 3071-3153 |
| TCGACCCAGGAGGGCCTGCGGATTCAAAACTGGTTGAGCGCCAAAGTGCGGAAGCAGCTGCGTCTCAAACCCTTTTATTTGGTC | 3154-3237 |
| CCGAAACATGCCAAAGAGGGTAACGGTTTTCAAGAGGAAACCTGGCGTTTGAGCTTCTCCCACATTGAGAAGGAGATTTTGAAC | 3238-3321 |
| AACCATGGTAAGTCCAAAACGTGCTGCGAGAATAAGGAAGAAAAATGTTGTCGCAAAGATTGTCTCAAATTGATGAAATATTTG | 3322-3405 |
| CTGGAACAACTCAAAGAGCGTTTTAAGGACAAGAAGCATCTCGACAAGTTCTCCTCGTATCACGTCAAGACCGCCTTCTTTCAT | 3406-3489 |
| GTCTGTACGCAGAACCCGCAAGATAGCCAGTGGGATCGCAAGGACTTGGGGTTGTGTTTTGACAATTGCGTCACCTATTTCTTG | 3490-3573 |
| CAATGTTTGCGGACCGAGAAATTGGAGAACTACTTTATTCCAGAATTCAACTTGTTTTCCTCGAATCTGATTGACAAACGCTCC | 3574-3657 |
| AAAGAGTTTCTGACGAAGCAGATTGAATACGAGCGTAACAATGAGTTTCCGGTCTTTGACGAGTTTTGAAAGCTTGAGATGGTG | 3658-3741 |
| AGCAAGGGCGAGGAGGATAACATGGCCATCATCAAGGAGTTCATGCGCTTCAAGGTGCACATGGAGGGCTCCGTGAACGGCCAC | 3742-3825 |
| GAGTTCGAGATCGAGGGCGAGGGCGAGGGCCGCCCCTACGAGGGCACCCAGACCGCCAAGCTGAAGGTGACCAAGGGTGGCCCC | 3826-3909 |
| CTGCCCTTCGCCTGGGACATCCTGTCCCCTCAGTTCATGTACGGCTCCAAGGCCTACGTGAAGCACCCCGCCGACATCCCCGAC | 3919-3993 |
| TACTTGAAGCTGTCCTTCCCCGAGGGCTTCAAGTGGGAGCGCGTGATGAACTTCGAGGACGGCGGCGTGGTGACCGTGACCCAG | 3994-4077 |
| GACTCCTCCCTGCAGGACGGCGAGTTCATCTACAAGGTGAAGCTGCGCGGCACCAACTTCCCCTCCGACGGCCCCGTAATGCAG | 4078-4161 |
| AAGAAGACCATGGGCTGGGAGGCCTCCTCCGAGCGGATGTACCCCGAGGACGGCGCCCTGAAGGGCGAGATCAAGCAGAGGCTG | 4162-4225 |
| AAGCTGAAGGACGGCGGCCACTACGACGCTGAGGTCAAGACCACCTACAAGGCCAAGAAGCCCGTGCAGCTGCCCGGCGCCTAC | 4226-4329 |
| AACGTCAACATCAAGTTGGACATCACCTCCCACAACGAGGACTACACCATCGTGGAACAGTACGAACGCGCCGAGGGCCGCCAC | 4330-4413 |
| TCCACCGGCGGCATGGACGAGCTGTACAAGTAGACTAGTTGCCTGGCGGCAGTAGCGCGGTGGTCCCACCTGACCCCATGCCGA | 4414-4497 |
| ACTCAGAAGTGAAACGCCGTAGCGCCGATGGTAGTGTGGGGTCTCCCCATGCGAGAGTAGGGAACTGCCAGGCATCAAATAAAA | 4498-4581 |
| CGAAAGGCTCAGTCGAAAGACTGGGCCTTTCGTTTTATCTGTTGTTTGTCGGTGAACGCTCTCCTGAGTAGGACAAATCCGCCG | 4582-4665 |
| GGAGCGGATTTGAACGTTGCGAAGCAACGGCCCGGAAGGGTGGCGGGCAGGACGCCCGCCATAAACTGCCAGGCATCAAATTAA | 4667-4749 |
| GCAGAAGGCCATCCTGACGGATGGCCTTTTTTCTAGAGTCGACCACCAAGGGCACCATCTCTGCTTGGGCCACCCCGTTGGCCG | 4750-4833 |
| CAGCCAGCTCGCTGAGAGCCGTGAACGACAGGGCGAACGCCAGCCCGCCGACGGCGAGGGTTCCGACCGCTGCAACTCCCGGTG | 4834-4917 |
| CAACCTTGTCCCGGTCTATTCTCTTCACTGCACCAGCTCCAATCTGGTGTGAATGCCCCTCGTCTGTTCGCGCAGGCGGGGGGC | 4918-5001 |
| TCTATTCGTTTGTCAGCATCGAAAGTAGCCAGATCAGGGATGCGTTGCAACCGCGTATGCCCAGGTCAGAAGAGTCGCACAAGA | 5002-5085 |
| GTTGCAGACCCCTGGAAAGAAAAATGGCCAGAGGGCGAAAACACCCTCTGACCAGCGGAGCGGGCGACGGGAATCGAACCCGCG | 5086-5169 |
| TAGCTAGTTTGGAAGAATGGGTGTCTGCCGACCACATATGGGCCGGTCAAGATAGGTTTTTACCCCCTCTCGGCTGCATCCTCT | 5170-5253 |
| AAGTGGAAAGAAATTGCAGGTCGTAGAAGCGCGTTGAAGCCTGAGAGTTGCACAGGAGTTGCAACCCGGTAGCCTTGTTCACGA | 5254-5337 |
| CGAGAGGAGACCTAGTTGGCACGTCGCGGATGGGGATCGCTGAAGACTCAGCGCAGCGGGAGGATCCAAGCCTCATACGTCAAC | 5338-5421 |
| CCGCAGGACGGTGTGAGGTACTACGCGCTGCAGACCTACGACAACAAGATGGACGCCGAAGCCTGGCTCGCGGGCGAGAAGCGG | 5422-5505 |
| CTCATCGAGATGGAGACCTGGACCCCTCCACAGGACCGGGCGAAGAAGGCAGCCGCCAGCGCCATCACGCTGGAGGAGTACACC | 5506-5589 |
| CGGAAGTGGCTCGTGGAGCGCGACCTCGCAGACGGCACCAGGGATCTGTACAGCGGGCACGCGGAGCGCCGCATCTACCCGGTG | 5590-5673 |
| CTAGGTGAAGTGGCGGTCACAGAGATGACGCCAGCTCTGGTGCGTGCGTGGTGGGCCGGGATGGGTAGGAAGCACCCGACTGCC | 5674-5757 |
| CGCCGGCATGCCTACAACGTCCTCCGGGCGGTGATGAACACAGCGGTCGAGGACAAGCTGATCGCAGAGAACCCGTGCCGGATC | 5768-5841 |
| GAGCAGAAGGCAGCCGATGAGCGCGACGTAGAGGCGCTGACGCCTGAGGAGCTGGACATCGTCGCCGCTGAGATCTTCGAGCAC | 5842-5925 |
| TACCGGATCGCGGCATACATCCTGGCGTGGACGAGCCTCCGGTTCGGAGAGCTGATCGAGCTTCGCCGCAAGGACATCGTGGAC | 5926-6009 |
| GACGGCATGACGATGAAGCTCCGGGTGCGCCGTGGCGCTTCCCGCGTGGGGAACAAGATCGTCGTTGGCAACGCCAAGACCGTC | 6010-6093 |
| CGGTCGAAGCGTCCTGTGACGGTTCCGCCTCACGTCGCGGAGATGATCCGAGCGCACATGAAGGACCGTACGAAGATGAACAAG | 6094-6177 |
| GGCCCCGAGGCATTCCTGGTGACCACGACGCAGGGCAACCGGCTGTCGAAGTCCGCGTTCACCAAGTCGCTGAAGCGTGGCTAC | 6178-6261 |
| GCCAAGATCGGTCGGCCGGAACTCCGCATCCACGACCTCCGCGCTGTCGGCGCTACGTTCGCCGCTCAGGCAGGTGCGACGACC | 6262-6345 |
| AAGGAGCTGATGGCCCGTCTCGGTCACACGACTCCTAGGATGGCGATGAAGTACCAGATGGCGTCTGAGGCCCGCGACGAGGCT | 6346-6429 |
| ATCGCTGAGGCGATGTCCAAGCTGGCCAAGACCTCCTGAAACGCAAAAAGCCCCCCTCCCAAGGACACTGAGTCCTAAAGAGGG | 6430-6513 |
| GGGTTTCTTGTCAGTACGCGAAGAACCACGCCTGGCCGCGAGCGCCAGCACCGCCGCTCTGTGCGGAGACCTGGGCACCAGCCC | 6514-6597 |
| CGCCGCCGCCAGGAGCATTGCCGTTCCCGCCAGCTGAGTTCTGTTGTGCGCCGCCTATGTAGAGCTGGTCGTTGTAGGTCCGA | 6598-6680 |
| TCTCCAGGCGACTTTCCGGCGACGCTGAGGATGTCGATCACAGAGCCTCCGGGACCGCCGGTTGCGGTCAAACCTGACCATCC | 6681-6763 |
| GACAGCGGACGCCGTGGTGTTTCCTCCAGGGCCTCCGGCCTTGCCTGAGAATACAGAGCCAGCTCCCGCTGCGCCTCCAGCTC | 6764-6846 |
| CGACGAGCCCGGTGATCGTCTTGGTCGACCTGCAGGCATGCAAAAGCTGATCCTTGCCGAGCTGGGATGGAAGCCCGGCCGAC | 6847-6929 |
| CCACCCTGGAGGAGATGATCGAGGATGCCAGGGCCTTTCACGCCCGCCGCTGCTGAGCGTCCGCCGCCGGGCCCGCACCGCCG | 6830-7012 |
| TCGGCCGGCCCGCTCCGGGCTCGCAGCAGCGGGCTTCGGCGCGGGCCCGGGGCTCCCGGGCCGCCGGGCGGGGCTCCGCCCGG | 7013-7095 |
| CGGCCGCCGGGGGCCGGGGGCGGCGCCGGGCGGCCCGGGGCGTCAGGCGCCGGGGGCGGTGTCCGGCGGCCCCCAGAGGAACT | 7096-7178 |
| GCGCCAGTTCCTCCGGATCGGTGAAGCCGGAGAGATCCAGCGGGGTCTCCTCGAACACCTCGAAGTCGTGCAGGAAGGTGAAG | 7179-7261 |
| GCGAGCAGTTCGCGGGCGAAGTCCTCGGTCCGCTTCCACTGCGCCCCGTCGAGCAGCGCGGCCAGGATCTCGCGGTCGCCCCG | 7262-7344 |
| GAAGGCGTTGAGATGCAGTTGCACCAGGCTGTAGCGGGAGTCTCCCGCATAGACGTCGGTGAAGTCGACGATCCCGGTGACCT | 7345-7427 |
| CGGTCGCGGCCAGGTCCACGAAGATGTTGGTCCCGTGCAGGTCGCCGTGGACGAACCGGGGTTCGCGGCCGGCCAGCAGCGTG | 7428-7510 |
| TCCACGTCCGGCAGCCAGTCCTCCAGGCGGTCCAGCAGCCGGGGCGAGAGGTAGCCCCACCCGCGGTGGTCCTCGACGGTCGC | 7511-7593 |
| CGCGCGGCGTTCCCGCAGCAGTTCCGGGAAGACCTCGGAATGGGGGGTGAGCACGGTGTTCCCGGTCAGCGGCACCCTGTGCA | 7594-7676 |
| GCCGGCCGAGCACCCGGCCGAGTTCGCGGGCCAGGGCGAGCAGCGCGTTCCGGTCGGTCGTGCCGTCCATCGCGGACCGCCAG | 7677-7759 |
| GTGGTGCCGGTCATCCGGCTCATCACCAGGTAGGGCCACGGCCAGGCTCCGGTGCCGGGCCGCAGCTCGCCGCGGCCGAGGAG | 7760-7842 |
| GCGGGGCACCGGCACCGGGGCGTCCGCCAGGACCGCGTACGCCTCCGACTCCGACGCGAGGCTCTCCGGACCGCACCAGTGCT | 7743-7925 |
| CGCCGAACAGCTTGATCACCGGGCCGGGCTCGCCGACCAGTACGGGGTTGGTGCTCTCGCCGGGCACCCGCAGCACCGGCGGC | 7926-8008 |
| ACCGGCAGCCCGAGCTCCTCCAGGGCTCGGCGGGCCAGCGGCTCCCAGAATTCCTGGTCGTTCCGCAGGCTCGCGTAGGAATC | 8009-8091 |
| ATCCGAATCAATACGGTCGAGAAGTAACAGGGATTCTTGTGTCACAGCGGACCTCTATTCACAGGGTACGGGCCGGCTTAATT | 8092-8174 |
| CCGCACGGCCGGTCGCGACACGGCCTGTCCGCACCGCGGATCAGGCGTTGACGATGACGGGCTGGTCGGCCACGTCGGGGACG | 8175-8257 |
| TTCTCGGTGGTGCTGCGGTCGGGATCGCCAATCTCTACGGGCCGACCGAGGCGACGGTGTACGCCACCGCCTGGTTCTGCGAC | 8258-8340 |
| GGCGAGGCGCCGTCCCAGGCCCCGCCGATCCCCGTCCCCCGCGTCGTCGAGCGCGGTGCCGACGACACCGCCGCGTGGCTCGT | 8341-8423 |
| CACGGAGGCCGTCCCCGGCGTCGCGGCGGCCGAGGAGTGGCCCGAGCACCAGCGGTTCGCCGTGGTCGAGGCGATGGCGGAGC | 8424-8506 |
| TGGCCCGCGCCCTCCACGAGCTGCCCGTGGAGGACTGCCCCTTCGACCGGCGCCTCGACGCGGCGGTCGCCGAGGCCCGGCGG | 8507-8589 |
| AACGTCGCCGAGGGCCTGTGGACCTCGACGACCTGCAGGCATGCAAGCTAGCTTTTGTTATCCGCTCACAATTCCACACAACA | 8590-8672 |
| TACGAGCCGGAAGCATAAAGTGTAAAGCCTGGGGTGCCTAATGAGTGAGCTAACTCACATTAATTGCGTTGCGCTCACTGCCC | 8673-8755 |
| GCTTTCCAGTCGGGAAACCTGTCGTGCCAGCTGCATTAATGAATCGGCCAACGCGCGGGGAGAGGCGGTTTGCGTATTGGGCG | 8756-8838 |
| CTCTTCCGCTTCCTCGCTCACTGACTCGCTGCGCTCGGTCGTTCGGCTGCGGCGAGCGGTATCAGCTCACTCAAAGGCGGTAA | 8839-8921 |
| TACGGTTATCCACAGAATCAGGGGATAACGCAGGAAAGAACATGTGAGCAAAAGGCCAGCAAAAGGCCAGGAACCGTAAAAAG | 8922-9004 |
| GCCGCGTTGCTGGCGTTTTTCCATAGGCTCCGCCCCCCTGACGAGCATCACAAAAATCGACGCTCAAGTCAGAGGTGGCGAAA | 9005-9087 |
| CCCGACAGGACTATAAAGATACCAGGCGTTTCCCCCTGGAAGCTCCCTCGTGCGCTCTCCTGTTCCGACCCTGCCGCTTACCG | 9088-9170 |
| GATACCTGTCCGCCTTTCTCCCTTCGGGAAGCGTGGCGCTTTCTCAATGCTCACGCTGTAGGTATCTCAGTTCGGTGTAGGTC | 9171-9253 |
| GTTCGCTCCAAGCTGGGCTGTGTGCACGAACCCCCCGTTCAGCCCGACCGCTGCGCCTTATCCGGTAACTATCGTCTTGAGTC | 9254-9336 |
| CAACCCGGTAAGACACGACTTATCGCCACTGGCAGCAGCCACTGGTAACAGGATTAGCAGAGCGAGGTATGTAGGCGGTGCTA | 9337-9419 |
| CAGAGTTCTTGAAGTGGTGGCCTAACTACGGCTACACTAGAAGGACAGTATTTGGTATCTGCGCTCTGCTGAAGCCAGTTACC | 9420-9502 |
| TTCGGAAAAAGAGTTGGTAGCTCTTGATCCGGCAAACAAACCACCGCTGGTAGCGGTGGTTTTTTTGTTTGCAAGCAGCAGAT | 9503-9585 |
| TACGCGCAGAAAAAAAGGATCTCAAGAAGATCCTTTGATCTTTTCTACGGGGTCTGACGCTCAGTGGAACGAAAACTCACGTT | 9586-9668 |
| AAGGGATTTTGGTCATGAGATTATCAAAAAGGATCTTCACCTAGATCCTTTTAAATTAAAAATGAAGTTTTAAATCAATCTAA | 9669-9751 |
| AGTATATATGAGTAAACTTGGTCTGACAGTTACCAATGCTTAATCAGTGAGGCACCTATCTCAGCGATCTGTCTATTTCGTTC | 8752-9834 |
| ATCCATAGTTGCCTGACTCCCCGTCGTGTAGATAACTACGATACGGGAGGGCTTACCATCTGGCCCCAGTGCTGCAATGATAC | 9835-9917 |
| CGCGAGACCCACGCTCACCGGCTCCAGATTTATCAGCAATAAACCAGCCAGCCGGAAGGGCCGAGCGCAGAAGTGGTCCTGCA | 9918-10000 |
| ACTTTATCCGCCTCCATCCAGTCTATTAATTGTTGCCGGGAAGCTAGAGTAAGTAGTTCGCCAGTTAATAGTTTGCGCAACGTT | 10001-10085 |
| GTTGCCATTGCTACAGGCATCGTGGTGTCACGCTCGTCGTTTGGTATGGCTTCATTCAGCTCCGGTTCCCAACGATCAAGGCGA | 10086-10169 |
| GTTACATGATCCCCCATGTTGTGCAAAAAAGCGGTTAGCTCCTTCGGTCCTCCGATCGTTGTCAGAAGTAAGTTGGCCGCAGTG | 10170-10253 |
| TTATCACTCATGGTTATGGCAGCACTGCATAATTCTCTTACTGTCATGCCATCCGTAAGATGCTTTTCTGTGACTGGTGAGTAC | 10254-10337 |
| TCAACCAAGTCATTCTGAGAATAGTGTATGCGGCGACCGAGTTGCTCTTGCCCGGCGTCAATACGGGATAATACCGCGCCACAT | 10338-10421 |
| AGCAGAACTTTAAAAGTGCTCATCATTGGAAAACGTTCTTCGGGGCGAAAACTCTCAAGGATCTTACCGCTGTTGAGATCCAGT | 10422-10505 |
| TCGATGTAACCCACTCGTGCACCCAACTGATCTTCAGCATCTTTTACTTTCACCAGCGTTTCTGGGTGAGCAAAAACAGGAAGG | 10506-10589 |
| CAAAATGCCGCAAAAAAGGGAATAAGGGCGACACGGAAATGTTGAATACTCATACTCTTCCTTTTTCAATATTATTGAAGCATT | 10590-10673 |
| TATCAGGGTTATTGTCTCATGAGCGGATACATATTTGAATGTATTTAGAAAAATAAACAAATAGGGGTTCCGCGCACATTTCCC | 10674-10757 |
| CGAAAAGTGCCACCTGACGTCTAAGAAACCATTATTATCATGACATTAACCTATAAAAATAGGCGTATCACGAGGCCCTTTCGT | 10758-10841 |
| C | 10842-10842 |

Knocking out Endogenous BCG Phosphodiesterase Genes and Intragenic Segments Encoding Phosphodiesterase Domains in Order to Increase CDN PAMP and DAMP Levels

Overexpression of CDNs by knocking out an endogenous BCG phosphodiesterase: WP_003414507

The BCG AHM08589.1 protein encodes a 316 amino acid endogenous bifunctional c-di-AMP and cGAMP phosphodiesterase in BCG that is 100% identical to the M tuberculosis Rv2837c over the C-terminal 316 amino acids (also called CdnP, CnpB, 3′-to-5′ oligoribonuclease A, bifunctional oligoribonuclease, or PAP phosphatase NrnA). The M. tuberculosis Rv2837c protein is known to hydrolyze both 3′-5′ c-di-AMP (bacterial PAMP molecule) and 2′-3′cGAMP (host DAMP molecule). Since the BCG protein is 100% identical over the C-terminal 315 amino acids, knockout (gene replacement) of the BCG AHM08589.1 protein will lead to increased levels of CDNs (3′-5′ c-di-AMP and 2′-3′cGAMP) in recombinant BCG.

SEQ ID No: 14

Bifunctional c-di-AMP and cGAMP phosphodiesterase CdnP (also called CnpB, 3′-to-5′ oligoribonuclease A, bifunctional oligoribonuclease, PAP phosphatase NrnA) from BCG, amino acid sequence (316 amino acids; BCG protein AHM08589.1; NCBI Reference DNA Sequence: CP003494.1 from BCG strain ATCC 35743; NCBI Reference Protein Identifier WP_003414507). A similar sequence is present in Mycobacterium tuberculosis as protein Rv2837c or MT2903, and in Mycobacterium bovis as protein Mb2862c.

MDAVGAAALLSAAARVGVVCHVHPDADTIGAGLALALVLDGCGKRVEVSF
AAPATLPESLRSLPGCHLLVRPEVMRRDVDLVVTVDIPSVDRLGALGDLT
DSGRELLVIDHHASNDLFGTANFIDPSADSTTTMVAEILDAWGKPIDPRV
AHCIYAGLATDTGSFRWASVRGYRLAARLVEIGVDNATVSRTLMDSHPFT
WLPLLSRVLGSAQLVSEAVGGRGLVYVVVDNREWVAARSEEVESIVDIVR
TTQQAEVAAVFKEVEPHRWSVSMRAKTVNLAAVASGFGGGGHRLAAGYTT
TGSIDDAVASLRAALG

SEQ ID No: 15

Bifunctional c-di-AMP and cGAMP phosphodiesterase gene, cdnP (also called cnpB or gene for 3′-to-5′ oligoribonuclease A, bifunctional oligoribonuclease, or PAP phosphatase NrnA) from BCG, DNA sequence (951 nucleotides [316 codons and 1 stop codon]; encodes BCG protein AHM08589.1; NCBI Reference Sequence: CP003494.1 from BCG strain ATCC 35743). A similar sequence is present in Mycobacterium tuberculosis encoding protein Rv2837c or MT2903, and in Mycobacterium bovis encoding protein Mb2862c.

GTGGACGCCGTCGGTGCCGCTGCGCTGTTGTCGGCCGCTGCCAGGGTCGG
GGTAGTCTGCCACGTCCACCCCGATGCCGACACCATCGGCGCCGGATTGG
CATTGGCATTGGTGTTGGACGGGTGCGGCAAGCGGGTAGAGGTCAGCTTT
GCCGCGCCGGCGACACTGCCCGAGTCGCTGCGTTCGCTGCCGGGCTGCCA
TCTGCTGGTCCGCCCTGAGGTGATGCGCCGCGATGTCGATTTGGTTGTGA
CTGTTGACATTCCGAGTGTTGATCGGCTCGGTGCTCTGGGCGATCTAACT
GATTCCGGGCGGGAGCTCCTGGTAATCGACCATCACGCCTCCAACGACCT
GTTCGGCACCGCGAATTTCATTGACCCGTCGGCGGATTCCACCACGACGA
TGGTTGCCGAGATCCTCGACGCGTGGGGGAAACCGATAGACCCGCGCGTC
GCGCACTGCATCTACGCCGGGTTGGCGACCGACACGGGGTCGTTTCGCTG
GGCCAGTGTGCGGGGGTATCGGCTGGCGGCGCGGCTGGTAGAGATCGGTG
TGGACAACGCCACCGTCAGCAGGACCTTGATGGACAGCCATCCCTTCACC
CTGGTTGCCGTTGTATCGCGGGTGTTGGGTTCGGCGCAGCTGGTGTCCGA
GGCGGTCGGTGGCCGCGGGCTGGTTTACGTCGTCGTCGACAACCGGGAGT
GGGTCGCTGCGCGCTCGGAGGAAGTGGAAAGCATCGTCGACATCGTCCGC
ACCACGCAACAAGCCGAGGTCGCGGCGGTGTTCAAGGAGGTCGAACCGCA
TCGGTGGTCGGTGTCGATGCGGGCTAAGACCGTGAATTTGGCCGCGGTTG
CCTCTGGGTTCGGTGGCGGTGGTCACCGGCTGGCCGCGGGGTATACGACC
ACCGGCTCGATCGACGACGCTGTGGCGTCGTTGCGCGCGGCGCTTGGTTA
G

SEQ ID No: 16

Bifunctional c-di-AMP and cGAMP phosphodiesterase CdnP (also called CnpB, Rv2837c, or MT2903, 3′-to-5′ oligoribonuclease A, bifunctional oligoribonuclease, PAP phosphatase NrnA) from Mycobacterium tuberculosis, amino acid sequence (336 amino acids; M. tuberculosis protein WP_003905944.1; NCBI/GenBank Reference Sequence: AL123456 from M. tuberculosis strain H37Rv). The M. tuberculosis protein has 20 additional amino acids at its N-terminus compared with the BCG protein (SEQ ID No: 14) which are underlined and boldfaced.

MTTIDPRSELVDGRRRAGARVDAVGAAALLSAAARVGVVCHVHPDADTIG
AGLALALVLDGCGKRVEVSFAAPATLPESLRSLPGCHLLVRPEVMRRDVD
LVVTVDIPSVDRLGALGDLTDSGRELLVIDHHASNDLFGTANFIDPSADS
TTTMVAEILDAWGKPIDPRVAHCIYAGLATDTGSFRWASVRGYRLAARLV
EIGVDNATVSRTLMDSHPFTWLPLLSRVLGSAQLVSEAVGGRGLVYVVVD
NREWVAARSEEVESIVDIVRTTQQAEVAAVFKEVEPHRWSVSMRAKTVNL
AAVASGFGGGGHRLAAGYTTTGSIDDAVASLRAALG

Overexpression of CDNs by knocking out an endogenous BCG phosphodiesterase domain: EAL domain of protein BCG_RS07340 (previously BCG_1416c). The BCG_RS07340 protein (SEQ ID No: 4) is encoded by the DNA sequence shown in SEQ ID No: 5. The BCG_RS07340 protein is 100% identical to the M. tuberculosis Rv1354c protein and is an endogenous CDN PDE in BCG. The full-length polypeptide is 623 amino acids in length, and it encodes a bifunctional diguanylate cyclase/diguanylate phosphodiesterase. The domain structure is: N-terminus-GAF-GGDEF-EAL-C-terminus as shown in FIG. 11. The GAF domain (approximately amino acids 1-190) is a regulatory domain which influences the activity of the other domains. The GGDEF domain (approximately amino acids 190-350) is a diguanylate cyclase catalyzing the reaction 2 GTP→4 c-di-GMP +2 pyrophosphates. The EAL domain (amino acids 354 to 623, highlighted in SEQ ID No: 4) is a diguanylate phosphodiesterase catalyzing the reaction c-di-GMP→4 2 GMP. As the EAL domain of this protein is known to cleave 3′-5′ c-di-GMP, knockout of this endogenous cyclic dinucleotide phosphodiesterase domain will increase the levels of c-di-GMP produced by BCG. Targeted knockout of the EAL domain may be accomplished by gene replacement of the full-length WT BCG_RS07340 gene with one which encodes only amino acids 1-353 (the GAF-GGDEF domains), that is truncating the coding sequence of the gene to exclude the sequences that encode amino acids 354-623 (shown as the underlined DNA sequence in SEQ ID No: 5) and including an appropriate stop codon and transcription termination sequence. Recombinant BCG lacking the EAL domain of BCG_RS07340 will lead to increased levels of the CDN PAMP c-di-GMP.

Overexpression of CDNs by knocking out an endogenous BCG phosphodiesterase: BCG AHM07112. The BCG_AHM07112 protein is an endogenous diguanylate phosphodiesterase in BCG (homologous the 307 amino acid M. tuberculosis Rv1357c protein). Some strains of BCG lack BCG AHM07112 altogether while others such as BCG Tice harbor it. Among the BCG strains that have this polypeptide, the protein may be 288 amino acids in length (such as in BCG ATCC 35743) or 307 amino acids in length (such as in BCG Pasteur 1173 P2). The BCG_AHM07112 protein from BCG ATCC 35743 is 288 amino acids in length and is 100% identical to the M tuberculosis Rv1357c protein over its C-terminal 287 amino acids. The domain structure of BCG AHM07112 is that of a single EAL domain (FIG. 11). As the M. tuberculosis Rv1357c protein is known to cleave 3′-5′ c-di-GMP, it is highly likely that the BCG protein performs the same reaction. Knockout of this endogenous cyclic dinucleotide phosphodiesterase in BCG is anticipated to increase the levels of c-di-GMP produced by BCG. Targeted knockout of the EAL domain may be accomplished by gene replacement of the full-length WT BCG AHM07112 gene and subsequent generation of an unmarked deletion.

SEQ ID No: 17

Diguanylate phosphodiesterase AHM07112.1 from BCG and other related mycobacteria, amino acid sequence (288 amino acids; GenBank Reference Sequence: CP003494.1; from BCG strain ATCC 35743). AHM07112.1 is 100% identical to the C-terminal 287 amino acids of the diguanylate phosphodiesterase of Mycobacterium tuberculosis protein Rv1357c or MT1400 and of Mycobacterium bovis as protein Mb1392c.

MIDYEEMFRGAMQARAMVANPDQWADSDRDQVNTRHYLSTSMRVALDRGE
FFLVYQPIIRLADNRIIGAEALLRWEHPTLGTLLPGRFIDRAENNGLMVP
LTAFVLEQACRHVRSWRDHSTDPQPFVSVNVSASTICDPGFLVLVEGVLG
ETGLPAHALQLELAEDARLSRDEKAVTRLQELSALGVGIAIDDFGIGFSS
LAYLPRLPVDVVKLGGKFIECLDGDIQARLANEQITRAMIDLGDKLGITV
TAKLVESPSQAARLRAFGCKAAQGWHFAKALPVDFFRE

SEQ No: 18

Diguanylate phosphodiesterase AHM07112.1 from BCG and other related mycobacteria, DNA sequence (867 nucleotides [288 codons, 1 stop codon]; GenBank Reference Sequence: CP003494.1; from BCG strain ATCC 35743). AHM07112.1 is 100% identical to the C-terminal 287 amino acids of the diguanylate phosphodiesterase of Mycobacterium tuberculosis protein Rv1357c or MT1400 and of Mycobacterium bovis as protein Mb1392c.

| | |
|---|---|
| 1 | ttgatcgact acgaagagat gtttaggggc gcgatgcaag cgcgagcgat ggtagccaat |
| 61 | cctgaccaat gggcggactc cgaccgcgac caggtcaaca ctcgccatta tctgtccact |
| 121 | tcgatgcgcg tggcactgga tcgcggtgaa ttcttcctcg tctaccagcc aatcatccgg |
| 181 | cttgccgaca accgcatcat cggcgccgag gccctgctgc gctgggaaca cccgacgttg |
| 241 | ggcacgctac tcccgggccg gttcatcgac cgtgccgaga acaacggact gatggtgccg |
| 301 | ctcacggcct tcgtgctcga gcaggcctgc cgccacgtcc gcagttggcg tgaccacagc |
| 361 | accgacccgc aaccgtttgt cagcgtcaac gtctccgcca gcaccatctg cgatcccggc |
| 421 | ttcctggtgc tggtcgaagg tgtgctcggc gaaaccggcc tgcccgccca tgccctgcag |
| 481 | ctcgaactgg ccgaggacgc gcgccttagc agagacgaga aggcggtgac caggctacaa |
| 541 | gaattgtccg ctctcggcgt cggcatcgcc atcgacgact tcggcattgg attctccagc |
| 601 | ctcgcctacc ttccccgcct ccccgtcgac gtggtcaaac tcgggggaaa gttcatcgag |
| 661 | tgcctcgatg gcgacattca agctcggctg gccaacgaac agatcacccg ggcaatgatc |
| 721 | gaccttggcg acaagctcgg tatcaccgtc actgcaaagc tagtcgaaag ccccagccaa |
| 781 | gccgcccggt tgcgcgcctt cggctgtaaa gccgcacaag gctggcactt tgccaaggca |
| 841 | ctgccggtcg actttttcag agagtag |

SEQ ID No: 19

Diguanylate phosphodiesterase Rv1357c or MT1400 from Mycobacterium tuberculosis and BCG Pasteur 1173 P2, amino acid sequence (307 amino acids. NCBI/GenBank Reference Sequence: AL123456 from M. tuberculosis strain H37Rv). The 19 amino acid N-terminal extension is present in the M. tuberculosis and in BCG Pasteur strain 1173 P2 but absent in several other BCG strains. The 19 amino acid N-terminal extension is underlined and boldfaced. The C-terminal 287 amino acids of M. tuberculosis Rv1357c are 100% identical to the BCG diguanylate phosphodiesterase AHM07112.1.

MDRCCQRATAFACALRPTKLIDYEEMFRGAMQARAMVANPDQWADSDRDQ
VNTRHYLSTSMRVALDRGEFFLVYQPIIRLADNRIIGAEALLRWEHPTLG
TLLPGRFIDRAENNGLMVPLTAFVLEQACRHVRSWRDHSTDPQPFVSVNV
SASTICDPGFLVLVEGVLGETGLPAHALQLELAEDARLSRDEKAVTRLQE
LSALGVGIAIDDFGIGFSSLAYLPRLPVDVVKLGGKFIECLDGDIQARLA
NEQITRAMIDLGDKLGITVTAKLVETPSQAARLRAFGCKAAQGWHFAKAL
PVDFFRE

The sequences referenced in the application are summarized in Table 1 below.

TABLE 1

| SEQUENCE NUMBER | DESCRIPTION |
|---|---|
| SEQ ID NO: 1 | Diadenylate cyclase DisA from BCG and other related |
| | mycobacteria, amino acid sequence |
| SEQ ID NO: 2 | Diadenylate cyclase disA from BCG and other related |
| | mycobacteria, DNA sequence |
| SEQ ID NO: 3 | Plasmid pSD5B-Pₕₛₚ₆₀::disA which overexpresses the disA |
| | gene, DNA sequence |
| SEQ ID NO: 4 | Bifunctional diguanylate cyclase/phosphodiesterase |
| | BCG_RS07340 from BCG and other related mycobacteria, |
| | amino acid sequence |
| SEQ ID NO: 5 | Bifunctional diguanylate cyclase/phosphodiesterase |
| | BCG_RS07340 from BCG and other related mycobacteria, |
| | DNA sequence |
| SEQ ID NO: 6 | Modified, bifunctional diguanylate cyclase/phosphodiesterase |
| | from BCG and other related mycobacteria lacking the EAL |
| | domain so that it functions as a monofunctional diguanylate |
| | cyclase, amino acid sequence |
| SEQ ID NO: 7 | Modified, bifunctional diguanylate cyclase/phosphodiesterase |
| | from BCG and other related mycobacteria lacking the EAL domain |
| | so that it functions as a monofunctional diguanylate cyclase, |
| | DNA sequence |
| SEQ ID NO: 8 | Cyclic GMP-AMP synthase DncV from Vibrio cholerae, |
| | amino acid sequence |
| SEQ ID NO: 9 | Cyclic GMP-AMP synthase dncV from Vibrio cholerae, |
| | DNA sequence |
| SEQ ID NO: 10 | Cyclic GMP-AMP synthase cGAS from Homo sapiens, |
| | amino acid sequence |
| SEQ ID NO: 11 | Cyclic GMP-AMP synthase cGAS from Homo sapiens, |
| | DNA sequence |
| SEQ ID NO: 12 | Cyclic GMP-AMP synthase cGAS gene from Homo sapiens |
| | with mycobacterial codon optimization, DNA sequence |
| SEQ ID NO: 13 | Plasmid pMH94H- Pₕₛₚ₆₀::disA::COcGAS::mCherry which |
| | overexpresses the disA gene, the codon-optimized human |
| | cGAS gene, and mCherry, DNA sequence |
| SEQ ID NO: 14 | Bifunctional c-di-AMP & cGAMP phosphodiesterase CdnP |
| | from BCG, amino acid sequence |
| SEQ ID NO: 15 | Bifunctional c-di-AMP & cGAMP phosphodiesterase CdnP |
| | from BCG, DNA sequence |
| SEQ ID NO: 16 | Bifunctional c-di-AMP & cGAMP phosphodiesterase CdnP |
| | from M. tuberculosis with 20 amino acid |
| | N-terminal extension, amino acid sequence. |
| SEQ ID NO: 17 | Diguanylate phosphodiesterase AHM07112.1 from BCG and |
| | other related mycobacteria, amino acid sequence |
| SEQ ID NO: 18 | Diguanyiate phosphodiesterase AHM07112.1 from BCG and |
| | other related mycobacteria, DNA sequence |
| SEQ ID NO: 19 | Diguanyiate phosphodiesterase Rv1357c or MT1400 from |
| | Mycobacterium tuberculosis and BCG Pasteur |
| | 1173 P2 with 19 amino acid N-terminal extension, |
| | amino acid sequence |

In some embodiments, the present invention relates to an expression cassette or expression vector comprising a nucleic acid sequence encoding a Rv1354c protein, or a functional part thereof, a nucleic acid sequence encoding a cyclic GMP-AMP synthase (DncV) protein, or a functional part thereof, a nucleic acid sequence encoding a cyclic GMP-AMP synthase (cGAS) protein, or a functional part thereof; or a combination thereof. In some embodiments, the expression vector or expression cassette further comprises a nucleic acid sequence encoding a DNA integrity scanning (disA) protein which functions as a diadenylate cyclase, or a functional part thereof. In some embodiments, the nucleic acid sequence encoding a Rv1354c protein does not contain a phosphodiesterase gene or phosphodiesterase domain. In some embodiments, the expression vector or expression cassette does not contain a phosphodiesterase gene or phosphodiesterase domain.

Methods for generating expression vectors and expression cassettes, transforming Mycobacteria and isolating the same have been described. In some embodiments, an expression vector or expression cassette of the invention comprises one or more regulatory sequences, e.g., a promoter and/or enhancer element, operably linked to a nucleic acid of the invention which controls or influences transcription of the nucleic acid. In some embodiments, an expression vector or expression cassette of the invention comprises one or more sequences operably linked to a nucleic acid of the invention which directs termination of transcription, post-transcriptional cleavage, and/or polyadenylation. In some embodiments, an expression vector or expression cassette of the invention comprises a variable length intervening sequence and/or a selectable marker gene operably linked to a nucleic acid of the invention.

In some embodiments, the present invention relates to a strain of Mycobacterium comprising an expression vector or expression cassette of the invention described herein. In some embodiments, the strain of Mycobacterium is Mycobacterium tuberculosis, Mycobacterium bovis, or a combination thereof. In some embodiments, the strain of Mycobacterium is BCG. In some embodiments, the strain comprises the plasmid of SEQ ID NO: 13.

In some embodiments, the present invention relates to a strain of Mycobacterium that expresses or overexpresses diadenylate cyclase and/or expresses or overexpresses one or more other cyclase genes or domains (e.g., those described herein). In some embodiments, the expression or overexpression results in release of one or more STING agonists (e.g., c-di-AMP, c-di-GMP, 2′-3′ cGAMP, and/or 3′-3′ cGAMP). In some embodiments, the present invention relates to a strain of Mycobacterium that expresses or overexpresses diadenylate cyclase and/or does not express a phosphodiesterase (PDE) that hydrolyzes STING agonists (e.g., contains a deletion of a PDE gene that hydrolyzes STING agonists). See, e.g., FIG. 12. In some embodiments, the strain of Mycobacterium is Mycobacterium tuberculosis, Mycobacterium bovis, or a combination thereof. In some embodiments, the strain of Mycobacterium is BCG.

Statistically Significant Anti-Tumor Effects with BCG-disA-OE in the Rat MNU Bladder Cancer Model

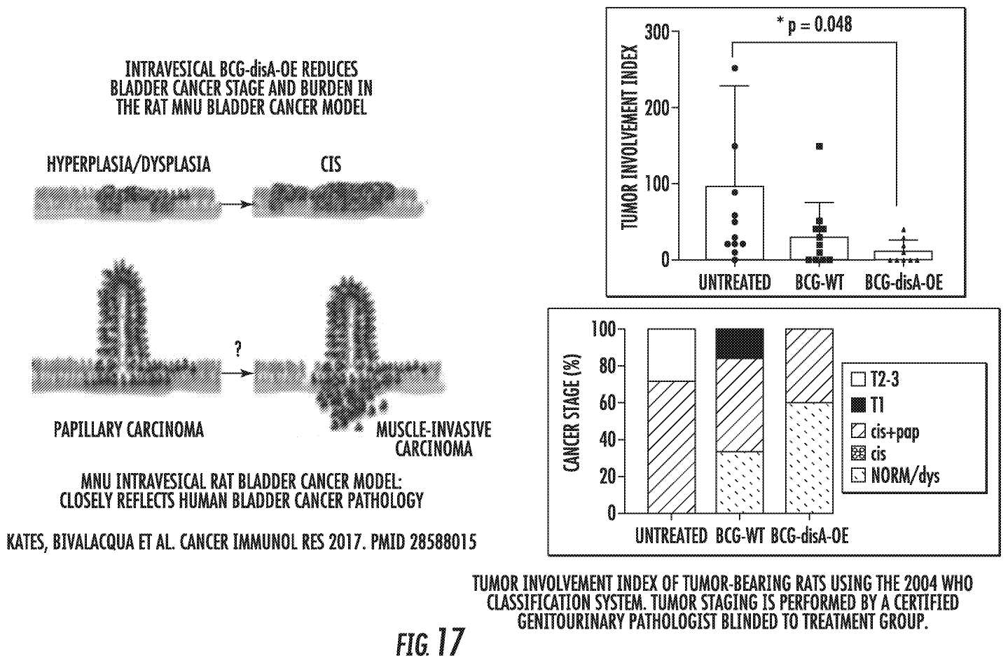

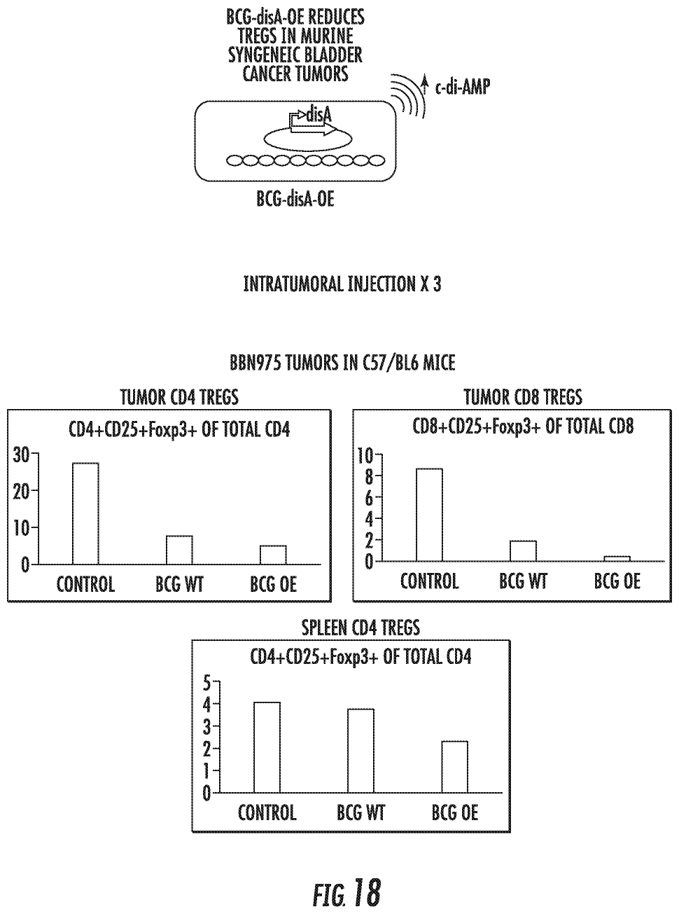

The rat MNU bladder cancer model is a validated model of bladder cancer in which administration of intravesical BCG can be shown to be therapeutic (FIG. 6 and Kates et al. PMID 28588015). The inventors extended their previous findings of the therapeutic effect of BCG-disA-OE versus BCG-WT which were shown in FIG. 7. The inventors have now performed the 16 week rat MNU model twice. FIG. 7 was based on Expt 1 and shows that BCG-disA-OE displays a trend towards a better outcome versus BCG-WT. After performing Expt 2 and combining its data with Expt 1, the inventor now show that BCG-disA-OE is statistically significantly superior to no treatment (p =0.048) whereas BCG-WT is not statistically significantly superior to no treatment (data shown in FIG. 17).

### Reduction of Tumor-Suppressive Treg Cells by BCG-disA-OE in a Murine Syngeneic Bladder Cancer Tumor Model.

In the MNU rat bladder cancer model the amount of bladder tissue at the end of the 16 week experiment is insufficient to perform flow cytometry. In order to study the cell population changes elicited by BCG-disA-OE the inventors developed a murine syngeneic bladder cancer tumor model using BBN975 cells. The model allows for large tumors (>1.5 cm in diameter) to develop on the mouse flank. Mice were treated with BCG-disA-OE and BCG-WT by intratumoral injection. As is shown in FIG. 18, the use of BCG-disA-OE led to reduced levels of tumor-associated CD4+ Treg cells, tumor-associated CD8+ Treg cells, and splenic CD4+ Treg cells.

BCG-disA-OE Delivers Sustained STING agonist from the Intracellular Compartment. Persistence of BCG in the Bladder.

Bowyer et al (The persistence of bacilli Calmette-Guerin in the bladder after intravesical treatment for bladder cancer. Brit J Urol. 1995; 75: 188-192. PMID 7850324) evaluated 125 bladder cancer patients from 1986-1992 who received intravesical BCG. Patients were asked to provide monthly urine samples which were then sent for mycobacterial culture. 90 patients survived and were compliant with the monthly urine samples. 4/90 patients (4.4%) had persistent BCG in their urine, one for up to 16.5 months. A fifth patient required a cystectomy 7 weeks after completing intravesical BCG treatments and was found to have microscopic evidence of acid-fast bacilli in the bladder by microscopy.

Durek et al. (The fate of bacillus Calmette-Guerin after intravesical instillation. J Urol. 2001; 165: 1765-1768. PMID 11342972) studied 49 patients with serial urine cultures following intravesical BCG. BCG was in the urine detected in 96.4% of the specimens after 2 hours and in 67.9% after 24 hours after instillation. The number of positive specimens decreased and it was 27.1% on day 7 immediately before the next instillation (FIG. 44). The investigators also evaluated bladder biopsies by PCR for mycobacterial DNA within 1 week after the 6^{th} instillation (instillations were given monthly). In 14 of 44 bladder biopsies (31.8%) mycobacterial ribosomal DNA was found. Additionally, positive PCRs for mycobacterial DNA was evident up to 24 months in between 4.2% and 37.5% of the investigated biopsies.

The fact that BCG is known to persist in bladder tissue represents an important advantage of the BCG-disA-OE strategy for STING agonist deliver in cancer. While numerous technologies have focus on generating small molecule STING agonists, such agents have relatively short exposure times. In contrast, as an intracellular microorganisms and as demonstrated by the Bowyer and Durek studies, BCG persists in cells and tissues for many weeks. The persistence of BCG-disA-OE in tissue offers sustained long-term deliver of the STING agonist in the tumor microenvironment

BCG-disA-OE is Safer than BCG-WT in Two Separate Mouse Models

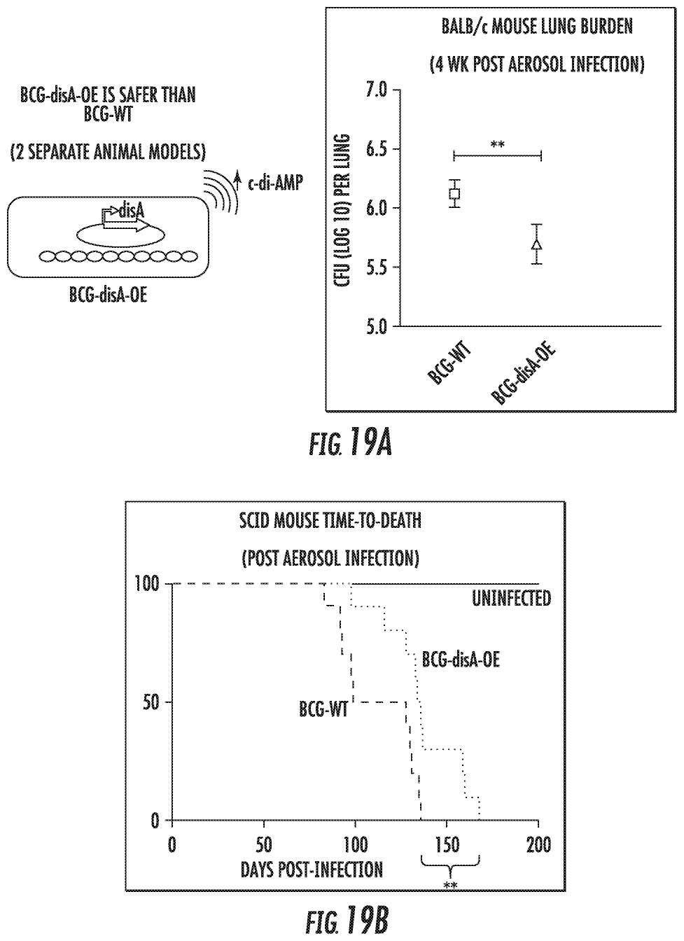

Intravesical BCG treatment in humans is associated with dysuria, fatigue, and malaise in treated patients. Additional more severe adverse effects are persistent cystitis with BCG and disseminated BCGosis. The patient safety of BCG was reviewed extensively in O'Donnell et al (Up-to-date, 2019). The incidence of dissemination of BCG into the bloodstream after intravesical instillation is estimated at 1/15,000 patients. To test the safety of BCG-disA-OE compared to BCG-WT the inventors used two mouse models of BCG infection where the BCG strains were aerosolized into the lungs of immunocompetent BABL/c mice or immunosuppressed SCID mice. As shown in FIG. 19, BCG-disA-OE was less capable of proliferating in immunocompetent mouse lungs than BCG-WT, and it was less lethal in a time-to-death assay in immunosuppressed mice.

BCG has been Shown to Elicit Trained Immunity which has been Associated with its Therapeutic Benefit in Solid and Liquid Tumors and for Diabetes. STING agonist Overexpressing BCG Strains Elicit Stronger Trained Immunity Changes than BCG-WT

Trained immunity. Trained immunity refers to the ability of one antigenic stimulus to elicit more potent immune responses to a second, different antigen. Trained immunity is antigen independent, based on heterologous CD4 and CD8 memory activation, cytokine mediated, and is associated with epigenetic and metabolic changes. BCG is a potent tool as the first antigenic stimulus to elicit trained immunity to subsequent antigenic stimuli such as tumors, viral infection, or drug-resistant bacterial infections (Netea et al. Trained immunity: a program of innate immune memory in health and disease. Science 2016. PMID 27102489; and Arts et al. BCG vaccination protects against experimental viral infection in humans through the induction of cytokines associated with trained immunity. Cell Host Microbe 2018. PMID 29324233).

BCG for solid and liquid tumors. BCG has a long history of therapeutic benefit as an immunotherapy for both solid and liquid tumors in humans (Hersh et al. BCG as adjuvant immunotherapy for neoplasia. Annu Rev Med 1977. PMID 324372). It has been used both systemically and intratumorally for malignancies that include melanoma, non-small cell lung cancer (NSCLC), and acute lymphoblastic leukemia (ALL). Recently there have been trials of BCG together with checkpoint inhibitors for forms of bladder cancer.

BCG for diabetes. BCG vaccination has recently been shown to have therapeutic benefits in glucose control for various forms of diabetes mellitus including Type 1 diabetes mellitus (Stienstra and Netea. Firing up glycolysis: BCG vaccination effects on Type 1 diabetes mellitus. Trends Endoc Metab 2018. PMID: 30327169). The effect is believed to be mediated by the trained immunity effects of BCG which have been shown to lead to epigenetic modifications which promote pro-inflammatory cytokine expression as well as the expression of metabolic enzymes such as those for glycolosis.

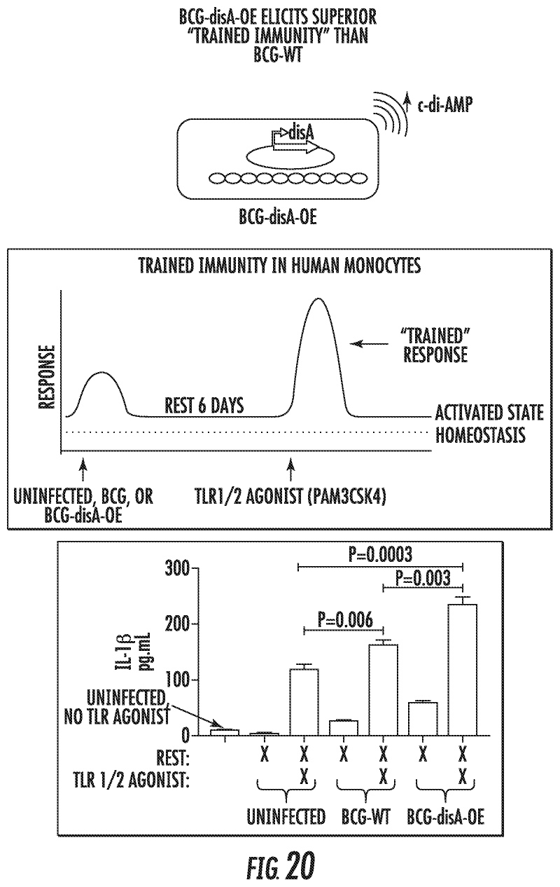

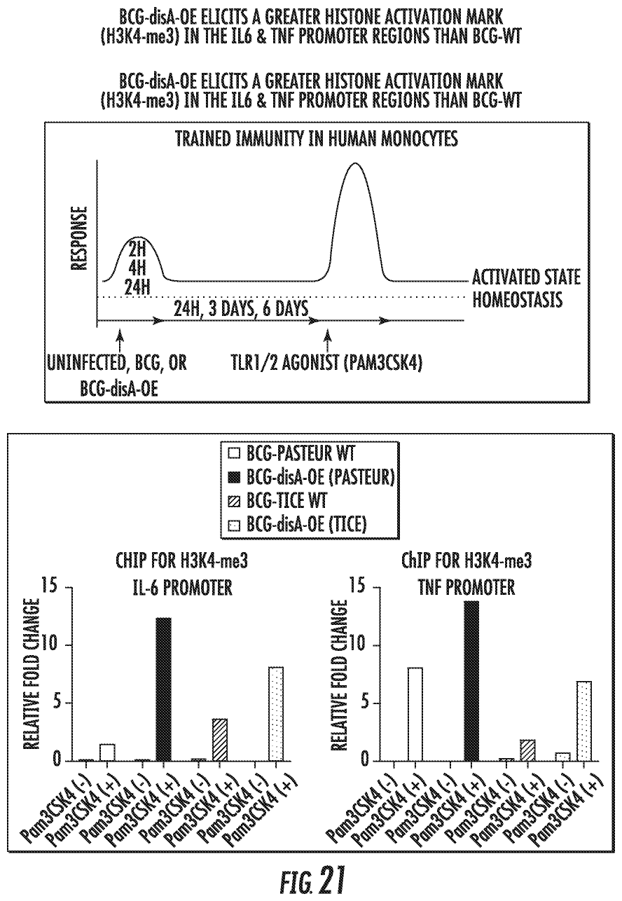

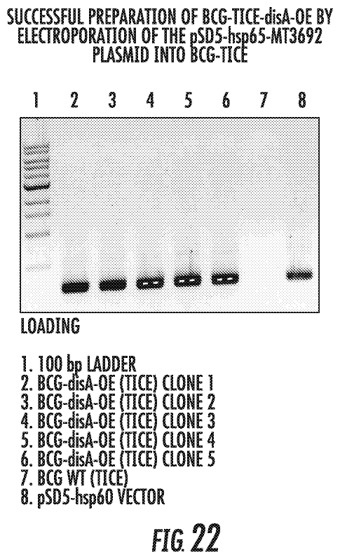

BCG-disA-OE and trained immunity. To investigate the ability of STING agonist overexpressing strains of BCG to stimulate trained immunity, the inventors tested the ability of BCG-WT versus BCG-disA-OE to elicit potentiation of second antigen stimulation in rested human monocytes following an exposure to the BCG strains six days prior. The first antigen was a BCG strain on day 0, and after six days of rest, the second antigen was the unrelated TLR-1/2 antigen PAM3CSK4. As may be seen in FIG. 20, upon receiving the second stimulus, the immune response tested (secretion of IL-1β) was potentiated by both BCG-WT and BCG-disA-OE, but the degree of stimulation by BCG-disA-OE was statistically significantly greater than that of either no BCG first stimulus or BCG-WT as the first stimulus. This reveals that STING overexpressing BCG strains such as BCG-disA-OE are a more potent stimulators of trained immunity than BCG-WT.

In a related experiment, the inventors conduced the same BCG-first stimulation/6 day rest/TLR-1, 2 second antigen stimulation with PAM3CSK4 experiment with human monocytes. At the end of the experiment cellular DNA was collected and subjected to chromatin immunoprecipitation (ChIP) using an antibody for the H3K4 histone methylation mark. The H3K4 mark is a known transcriptional activation mark. Upon quantitative PCR amplification of the IL-6 promoter region of the immunoprecipitated DNA, the results showed that BCG-Pasteur-disA-OE and BCG-Tice-disA-OE were statistically significantly more potent in eliciting the H3K4 mark in the IL-6 promoter (IL-6 is a pro-inflammatory cytokine) than their respective BCG-WT strains. These results show that STING overexpressing BCG strains such as BCG-disA-OE are a more potent stimulators of epigenetic changes associated with trained immunity than BCG-WT.

BCG-Tice-disA-OE Expresses Much Higher Levels of the disA Gene than BCG-WT

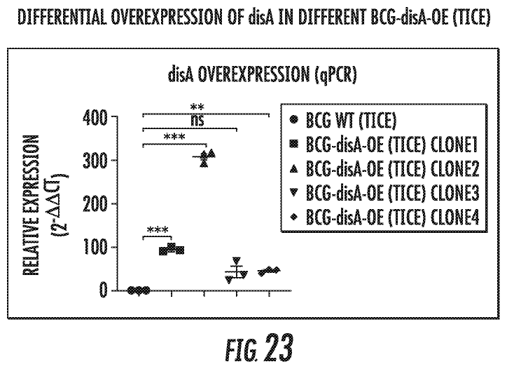

As may be seen in FIG. 23, the relative expression of BCG-Tice-disA-OE clone 2 (which was selected for seed-lot preparation and storage) was 300:1 using the 2^{-ΔΔCT} method of comparison. This indicates that disA is strongly overexpressed by being on a multicopy plasmid and driven by the M leprae hsp65 promoter in pSD5-hsp65-MT3692 plasmid. This strong overexpression leads to much higher levels of release of the STING agonist, c-di-AMP.

### STING Agonist Overexpression BCG Strains Such as BCG-disA-OE Elicit Pro-Inflammatory Changes In Signaling Pathways and Cytokine Secretion Profiles in Multiple Model Systems.

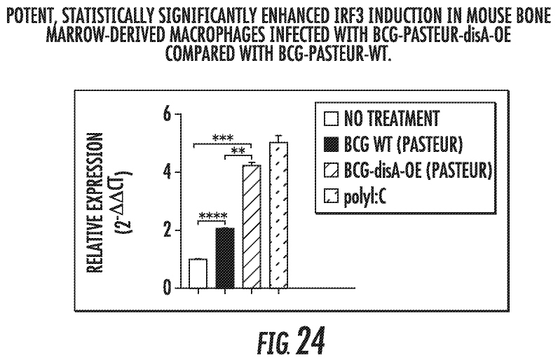

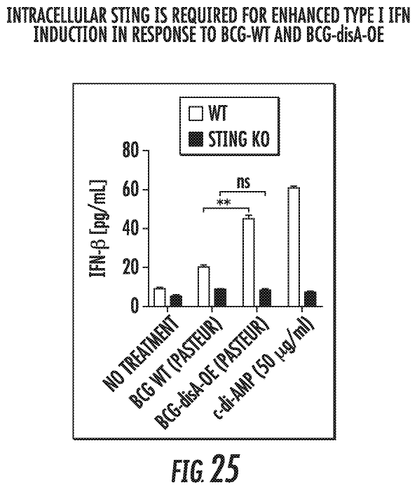

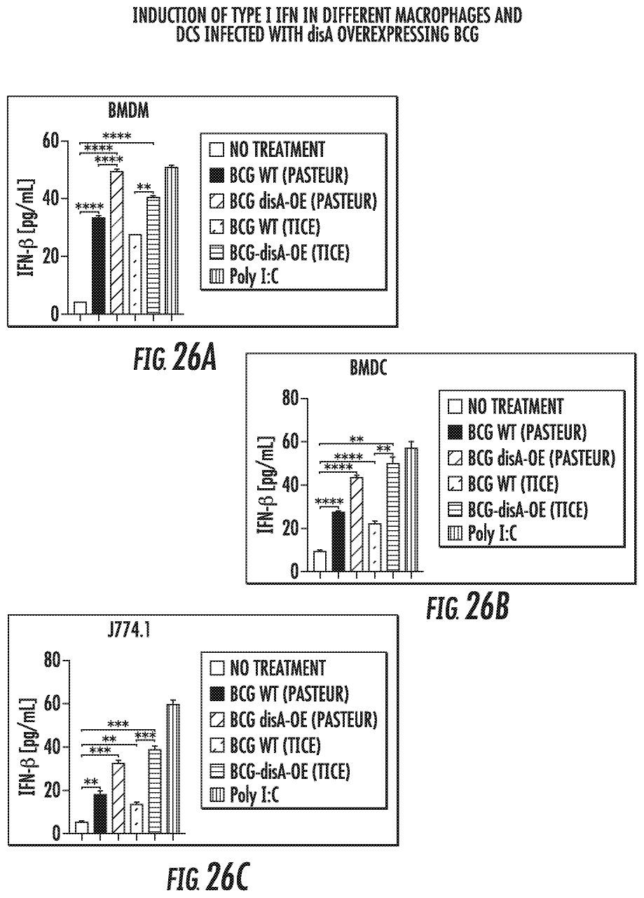

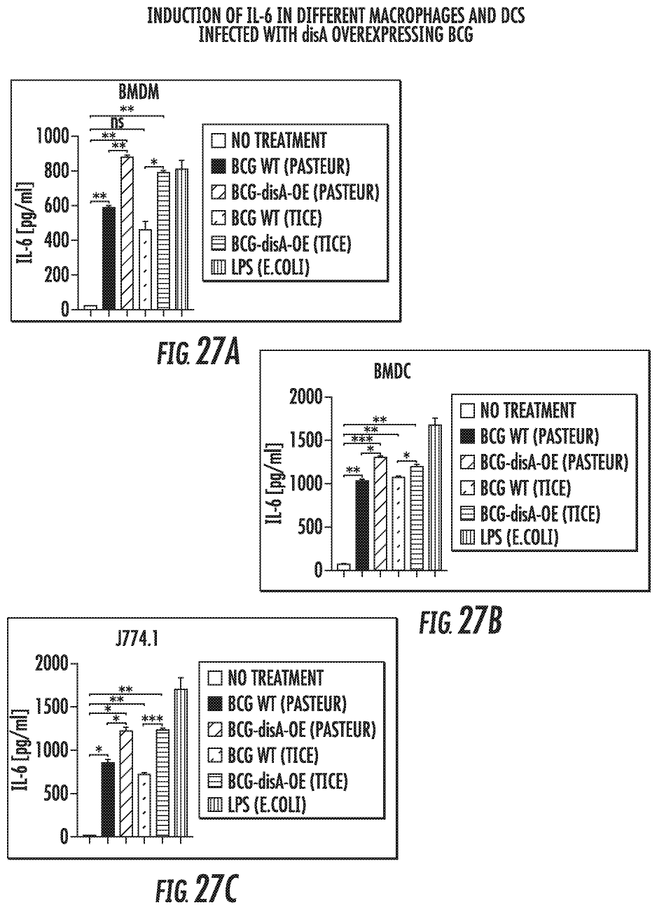

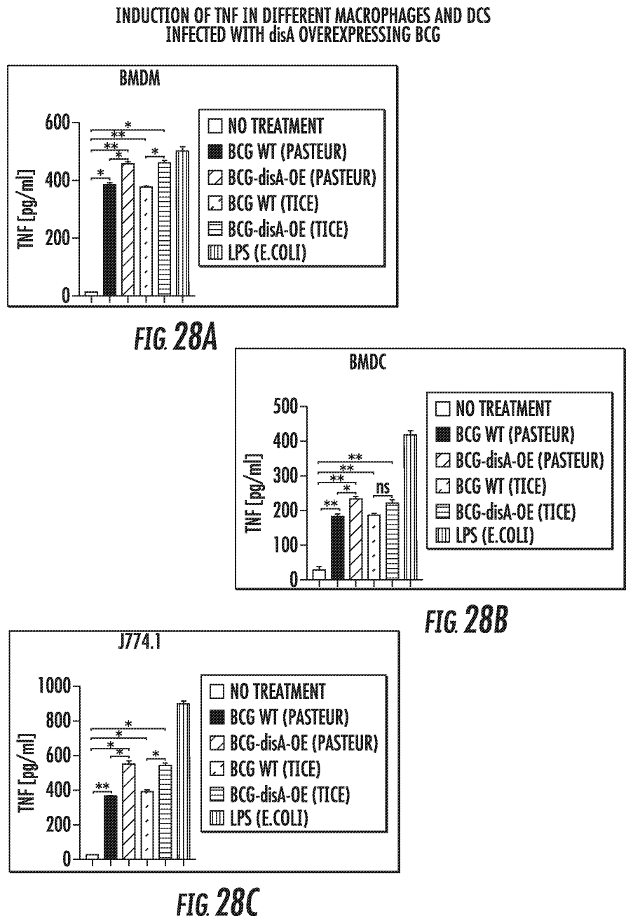

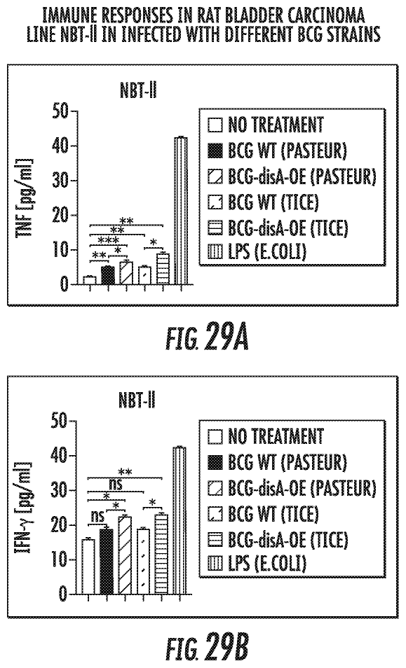

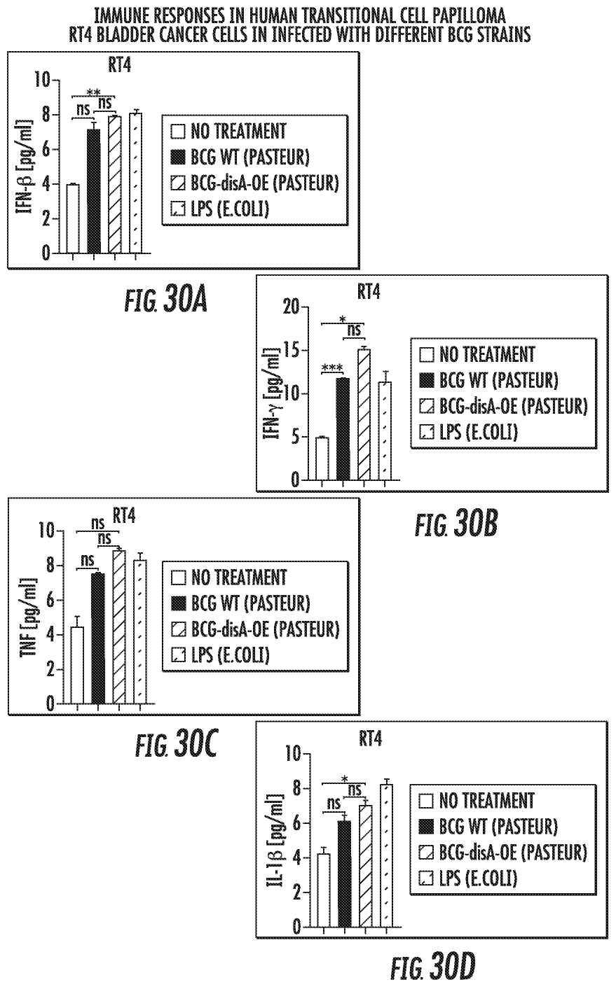

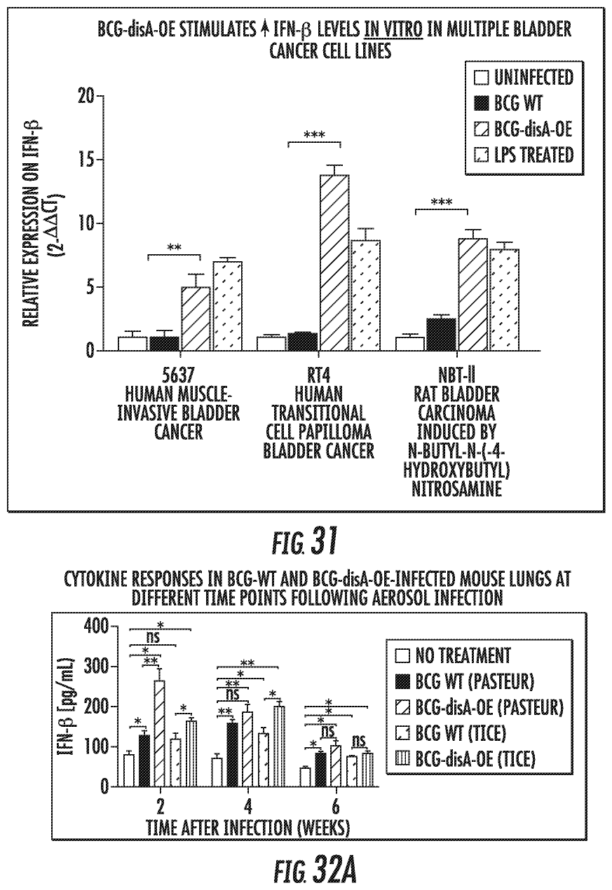

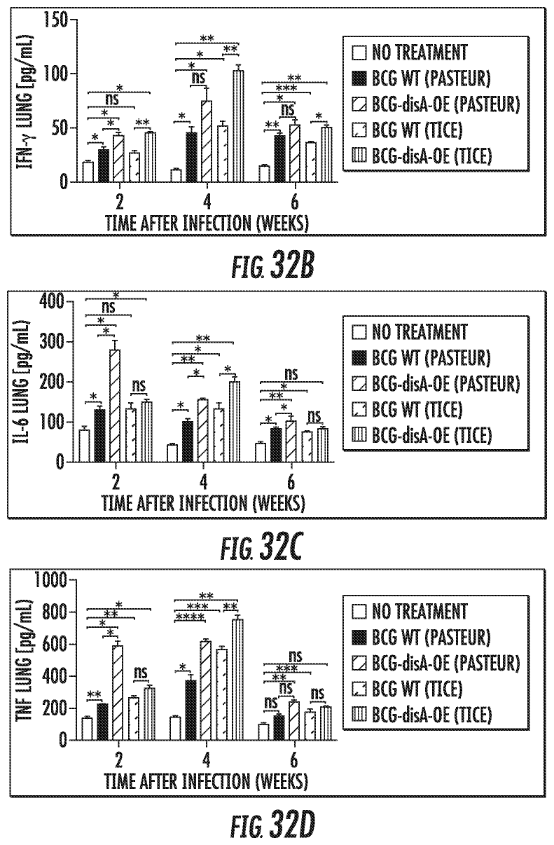

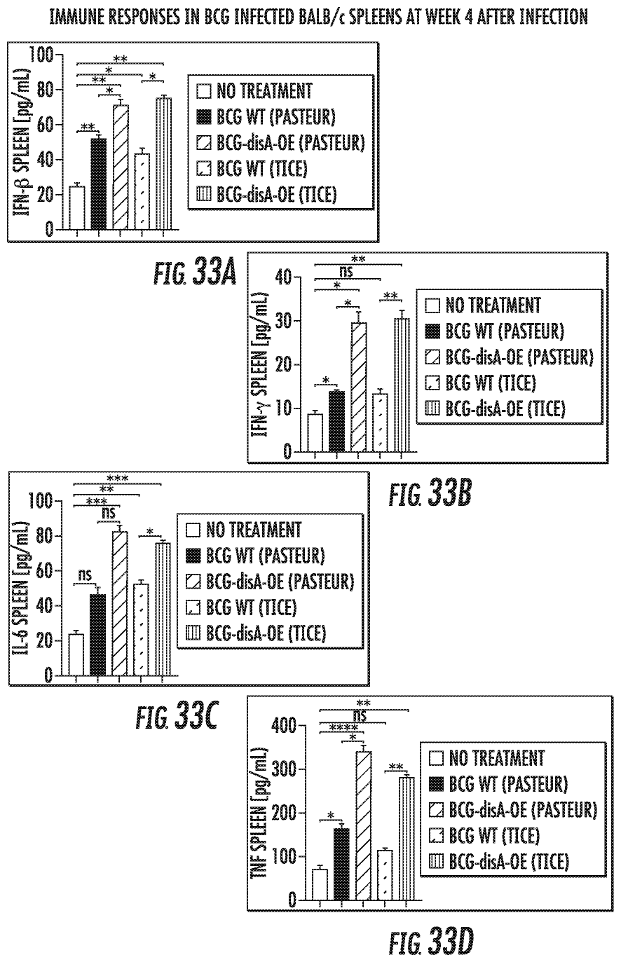

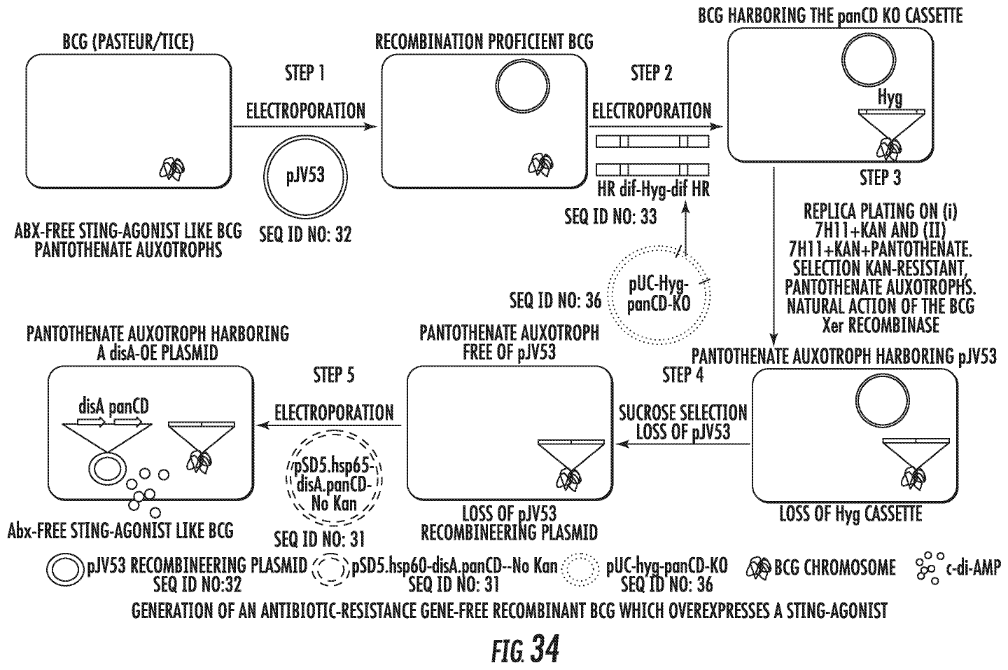

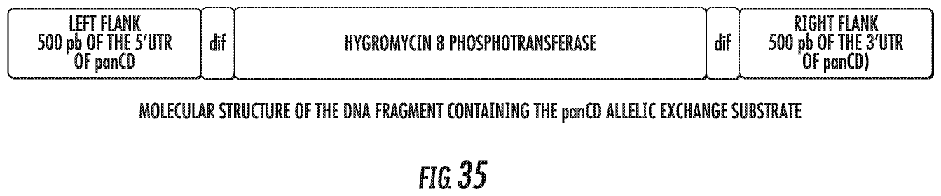

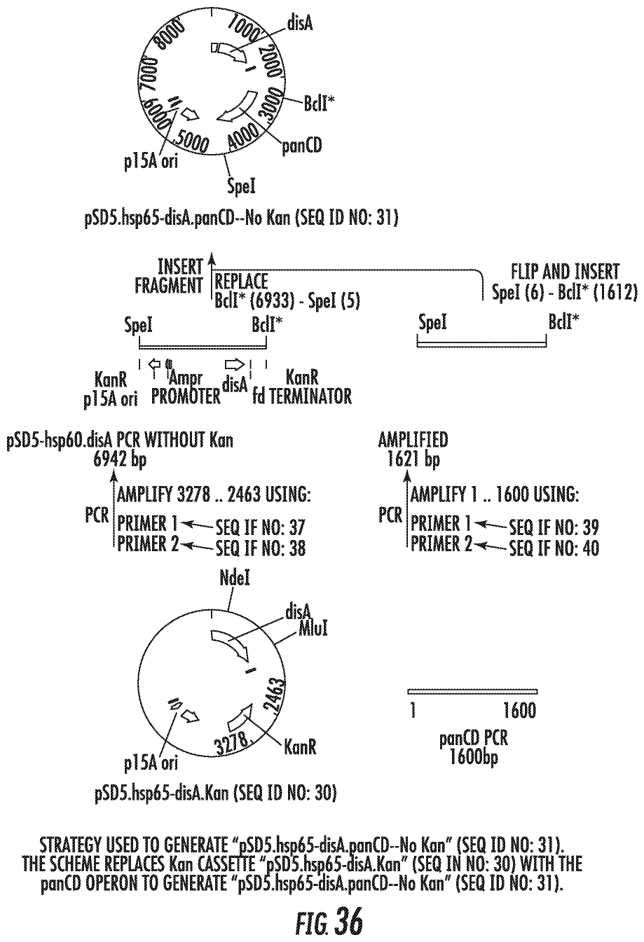

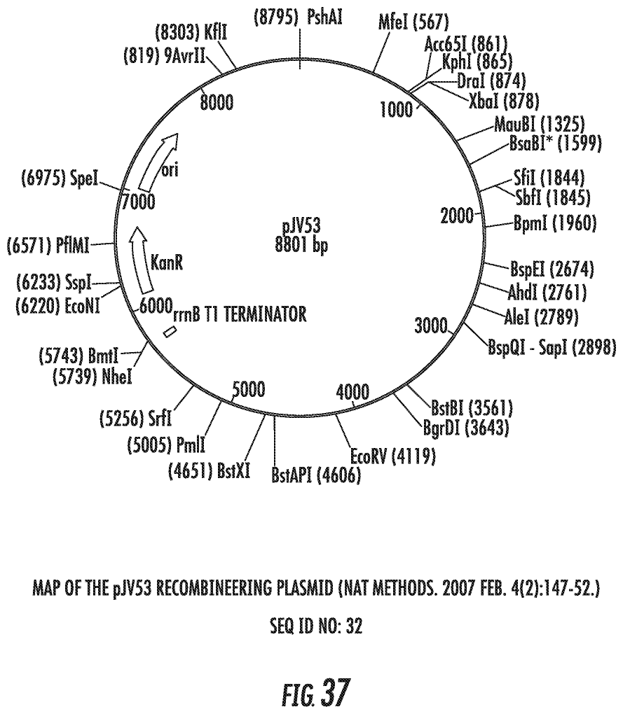

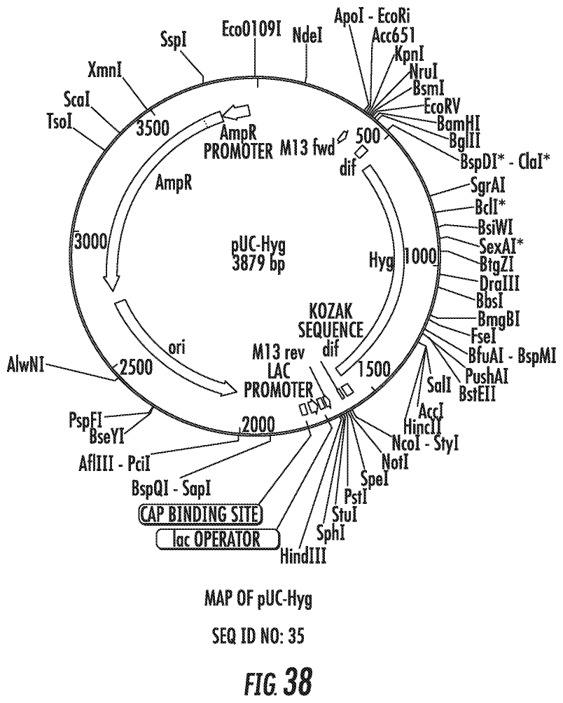

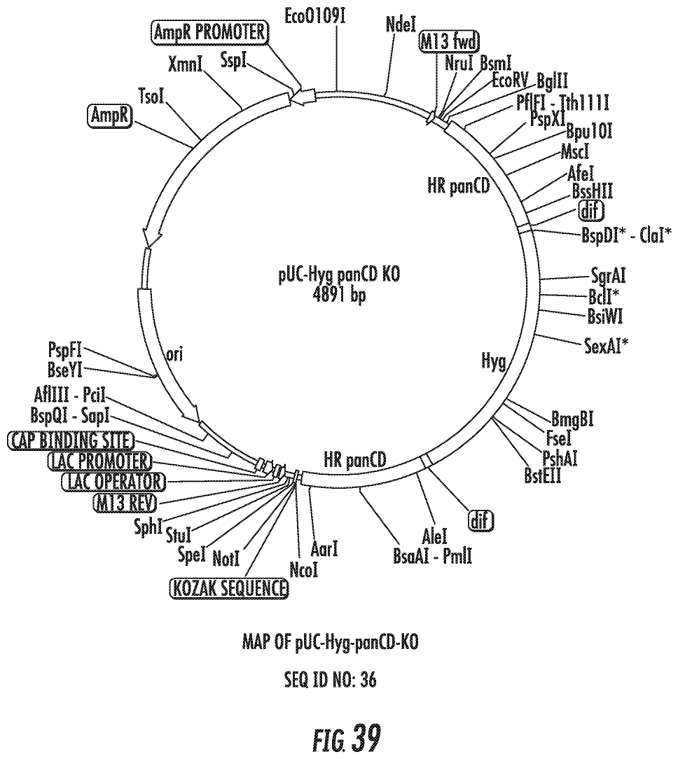

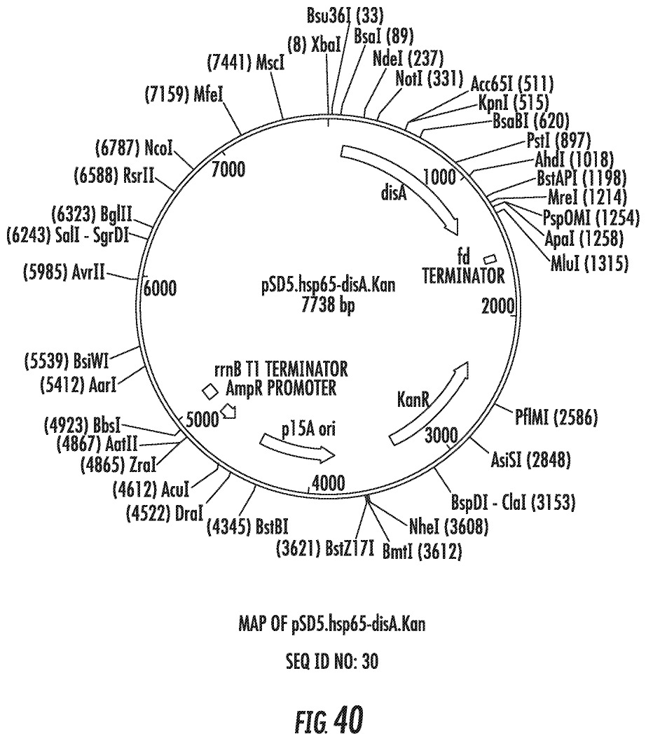

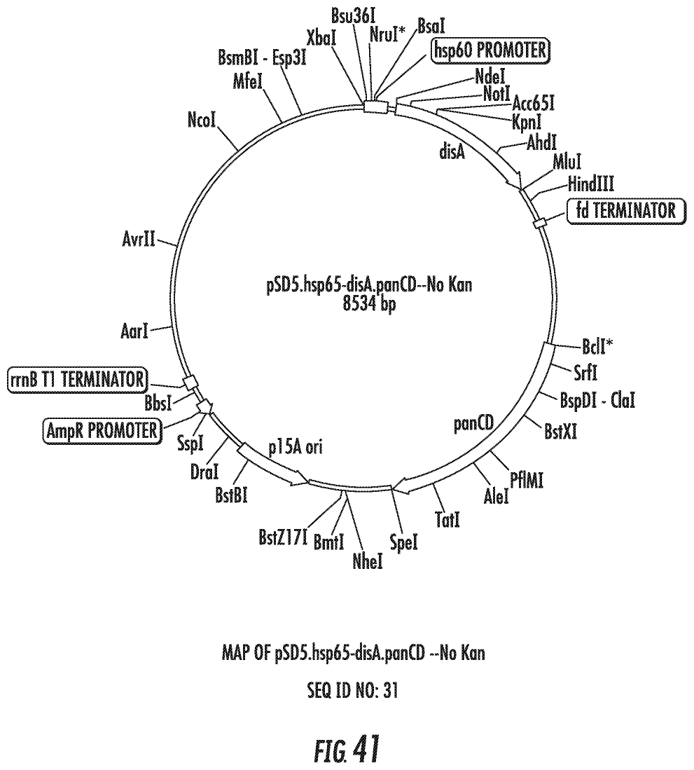

The inventors tested STING agonist overexpressing strains such as BCG-disA-OE compared to BCG-WT in multiple model systems to evaluate its relative capacity to elicit proinflammatory cytokine changes. BCG-disA-OE was statistically significantly superior than BCG-WT in the majority of their tests. And when the comparisons were not statistically significant, BCG-disA-OE gave the stronger of the two responses.
FIG. 25 also shows that the elevation of Type 1 IFN secretion in both BCG-disA-OE and BCG-WT is STING-dependent.
In summary, BCG-disA-OE is a more potent stimulator of pro-inflammatory cytokine expression and proinflammatory pathway induction than BCG-WT
The table below summarizes the data:

| | | | | |
|---|---|---|---|---|
| Mouse BMDM in vitro | IRF3 | qRT- | BCG-disA-OE > BCG- | FIG. 24 |
| | | PCR | WT | |
| Mouse BMDM, BMDC, | IFN-β, | ELISA | BCG-disA-OE > BCG- | FIG. 26 |
| J774 macrophage cell | | | WT | |
| line in vitro | | | | |
| Mouse BMDM; BMDC. | IL-6 | ELISA | BCG-disA-OE > BCG- | FIG. 27 |
| J774 macrophage cell | | | WT | |
| line in vitro | | | | |
| Mouse BMDM, BMDC, | TNF | ELISA | BCG-disA-OE > BCG- | FIG. 28 |
| J774 macrophage cell | | | WT | |
| line in vitro | | | | |
| Rat bladder cancer NBT-II | TNF, IFN-γ | ELISA | BCG-disA-OE > BCG- | FIG. 29 |
| line in vitro | | | WT | |
| Human bladder cancer | IFN-β, IFN-γ, | ELISA | BCG-disA-OE > BCG- | FIG. 30 |
| RT4 line in vitro | TNF, IL-1β | | WT | |
| 5637, RT4, NBT-II | IFN-β | qRT- | BCG-disA-OE > BCG- | FIG. 31 |
| bladder cancer cell | | PCR | WT | |
| lines In vitro | | | | |
| Mouse lungs in vivo | IFN-β, IFN-γ, | ELISA | BCG-disA-OE > BCG- | FIG. 32 |
| (different time | IL-6, TNF | | WT | |
| points) In vivo | | | | |
| Mouse spleens in vivo | IFN-β; IFN-γ; | ELISA | BCG-disA-OE > BCG- | FIG. 33 |
| (4 weeks). In vivo | IL-6; TNF | | WT | |

A Method to Produce an Antibiotic Gene Cassette-Free Recombinant BCG which Overexpresses a STING Agonist Biosynthetic Gene.

The disA-overexpressing plasmid pSD5-hsp65-MT3692 carries a Kan resistance gene cassette conferring resistance to the antibiotic kanamycin. The inventors disclose a method to generate an antibiotic gene cassette-free recombinant BCG which overexpresses a STING agonist biosynthetic gene.

The mycobacterial genetic operon panCD encodes for the biosynthetic gene panC (Pantoate-beta-alanine ligase gene) and panD (aspartate 1-decarboxylase gene). The gene products PanC and PanD are required for the biosynthesis of pantothenic acid also called vitamin B₅ (a B vitamin). Pantothenic acid, a water-soluble vitamin, is an essential nutrient for mycobacteria such as BCG. Animals require pantothenic acid in order to synthesize coenzyme-A (CoA), as well as to synthesize and metabolize proteins, carbohydrates, and fats. The anion is called pantothenate.
Genetic deletion of panCD in mycobacteria has been shown to yield mutant strains that can only grow in the presence of added pantothenate. As such they are auxotrophs for pantothenate. ΔpanCD mutants of Mycobacterium tuberculosis, have been shown to be highly attenuated in animal infection, being rapidly cleared, because of their inability to grow in mammalian tissues where pantothenate is not available to them.
The inventors disclose a detailed method for generating an unmarked (no antibiotic gene cassettes) ΔpanCD deletion mutant of BCG. This mutant will only be able to grow in the presence of pantothenate and would not be expected to survive during infection or be an effective delivery vector for STING agonist expression.
The inventors disclose a detailed method for generating a shuttle plasmid which harbors the mycobacterial panCD gene as well as an overexpression construct for the biosynthesis of STING agonists (such as the Phsp65::disA construct which overexpresses the disA gene and releases excess STING agonist, c-di-AMP). The shuttle plasmid is capable of replication in E. coli or in mycobacteria. It harbors an antibiotic cassette that can be conveniently removed by cleavage with a rare-cutting restriction enzyme and re-ligation. Alternatively, the shuttle plasmid may be generated by PCR amplification of the backbone of the plasmid excluding the antibiotic resistance cassette that generates unique restriction sites at the termini and ligating in a PCR product consisting of an amplified panCD operon with the same unique restriction sites at its termini. In either manner the antibiotic resistance gene-free shuttle plasmid (ligation product) may be electroporated into a BCG or E. coli auxotroph and selected for on pantothenate-free agar plates.

In the final manifestation of this disclosure, the inventors show a method to introduce the antibiotic-cassette-free plasmid harboring the mycobacterial panCD gene as well as an overexpression construct for the biosynthesis of STING agonists (such as the Phsp65::disA construct) into an unmarked BCG ΔpanCD mutant. The end result is a BCG strain that harbors no antibiotic resistance genes, and that strongly overexpresses a STING agonist biosynthetic gene(s). In a mammalian host or a human, such a BCG strain would be under strong selective pressure to retain the plasmid due to its requirement for panCD complementation from the plasmid.
In another manifestation of the disclosure, the panCD cassette and the construct for the biosynthesis of STING agonists (such as the Phsp65::disA construct) could be introduced into a chromosomally integrating vector such as pMH94. Using similar methods the antibiotic cassette could be eliminated from pMH94. Introduction of this chromosomally integrating plasmid into an unmarked BCG ΔpanCD mutant would also yield a BCG strain that harbors no antibiotic resistance genes, and that strongly overexpresses a STING agonist biosynthetic gene(s). A disadvantage of this strategy is that the overexpression construct would be in single copy on the bacterial chromosome, rather than being in multicopy on a plasmid, and this could result in lower levels of STING agonist release.

### BCG-Tice (ATCC 35743) is a Natural Pantothenate Auxotroph.

The inventors disclose that the Mycobacterium bovis BCG Tice strain (ATCC 35743) is a natural pantothenate auxotroph. This strain carries a 5 bp DNA insertion in its panC gene at base pairs 739-743. This insertion mutation changes leads to a frameshift mutation after the 246^{th} amino acid of PanC (wild type PanC is 309 amino acids in length). As a result of the 5 bp insertion mutation, the mutant PanC polypeptide in the Mycobacterium bovis BCG Tice strain (ATCC 35743) is comprised of 246 amino acids of the wild type PanC sequence at its N-terminus followed by a 478 amino acid nonsense polypeptide at its C-terminus. This mutant PanC polypeptide is highly unlikely to retain any functional pantoate-beta-alanine ligase activity (the normal enzymatic function of PanC). Additionally, The PanD polypeptide in BCG Tice (ATCC 35743) is highly unlikely to be translated because the stop codon for the panC gene (which overlaps with the ATG for panD translation initiation in the wild type sequence) is out of frame. Ribosomal termination of PanC translation is coupled with ribosomal initiation of PanD translation in the wild type panCD operon. Since there is no ribosomal termination immediately upstream of the panD start codon, ribosomal initiation of translation of the panD gene is highly unlikely to occur.

The inventors disclose that this natural auxotrophy enables the more rapid construction of an antibiotic gene cassette-free recombinant BCG which overexpresses a STING agonist biosynthetic gene.

The inventors disclose a method for introducing an antibiotic-cassette-free plasmid harboring the mycobacterial panCD gene as well as an overexpression construct for the biosynthesis of STING agonists (such as the Phsp65::disA construct) directly into BCG-Tice (ATCC 35743).

Please note that pSD5-hsp60-MT3692 is the same as pSD5-hsp65-MT3692. The inventors had previously referred to this same plasmid as pSD5-hsp60-MT3692. However, the actual promoter in this strain is the promoter for the hsp65 gene of M. leprae. Thus, the inventors may refer to the plasmid pSD-hsp60-MT3692 as pSD5-hsp65-MT3692.

In some embodiments, the present invention relates to a pharmaceutical composition comprising an expression vector, expression cassette, or strain of the invention described herein and a pharmaceutically acceptable carrier.

In some embodiments, the present invention relates to methods and/or compositions for treating and/or preventing cancer comprising administration of an expression vector, expression cassette, strain or pharmaceutical composition described herein to a subject. In some embodiments, the cancer is bladder cancer (e.g., non-muscle invasive bladder cancer (NMIBC)), breast cancer, or a solid tumor. Additional embodiments of the disclosure concern methods and/or compositions for treating and/or preventing a bladder cancer in which modulation of a Type 1 interferon (IFN) response is directly or indirectly related. In certain embodiments, individuals with a bladder cancer such as NMIBC are treated with a modulator of the Type 1 interferon response, and in specific embodiments an individual with bladder cancer is provided a modulator of expression Type 1 interferon expression, such as an inducer of its expression.

In certain embodiments, the level to which an inducer of Type 1 interferon expression increases Type 1 interferon expression may be any level so long as it provides amelioration of at least one symptom of bladder cancer, including non-muscle-invasive bladder cancer (NMIBC). The level of expression of Type 1 interferon may increase by at least 2, 3, 4, 5, 10, 25, 50, 100, 1000, or more fold expression compared to the level of expression in a standard, in at least some cases. An individual may monitor expression levels of Type 1 interferon using standard methods in the art, such as northern assays or quantitative PCR, for example.

An individual known to have bladder cancer, suspected of having bladder cancer, or at risk for having bladder cancer may be provided an effective amount of an inducer of Type 1 interferon expression, including a BCG strain of the present invention comprising an expression vector of the present invention. The expression vector expresses a RV1354c protein, or functional part thereof; a cyclic GMP-AMP synthase (DncV) protein, or functional part thereof; a cyclic GMP-AMP synthase (cGAS) protein, or functional part thereof; a DNA integrity scanning (disA) protein which functions as a denylate cyclase, or functional part thereof; or a combination thereof. It is preferred that a BCG strain of the present invention comprising an expression vector of the present invention be administered into the bladder of the subject and that the expressed protein (s) enhance Type 1 interferon expression in the bladder. Those at risk for bladder cancer may be those individuals having one or more genetic factors, may be of advancing age, and/or may have a family history, for example.

In particular embodiments of the disclosure, an individual is given an agent for bladder cancer therapy in addition to the one or more inducers of Type 1 interferon of the present invention. Such additional therapy may include intravesical chemotherapies such as mitomycin C, cyclophosphamide, or a combination thereof, for example. When combination therapy is employed with one or more inducers of Type 1 interferon (such as a BCG strain expressing one or more of the following proteins: a RV1354c protein, or functional part thereof; a cyclic GMP-AMP synthase (DncV) protein, or functional part thereof; a cyclic GMP-AMP synthase (cGAS) protein, or functional part thereof; a DNA integrity scanning (disA) protein which functions as a denylate cyclase, or functional part thereof) the additional therapy may be given prior to, at the same time as, and/or subsequent to the inducer of Type 1 interferon.

In some embodiments, an expression vector, expression cassette, strain, pharmaceutical composition, and/or method of the invention described herein has increased safety, increased tolerability (e.g., decreased dysuria, urgency, or malaise), and/or decreased likelihood to cause infection in the bloodstream or disseminated bloodstream infection compared to non-recombinant BCG.

In some embodiments, the present invention relates to a method of treating and/or preventing cancer, comprising administering to a subject an expression vector, expression cassette, strain, and/or pharmaceutical composition of the invention described herein, wherein the administration results in an increased safety profile, increased tolerability (e.g., decreasing dysuria, urgency, or malaise), and/or decreased likelihood of infection in the bloodstream or disseminated bloodstream infection compared to non-recombinant BCG. In some embodiments, the cancer is, for example, bladder cancer (e.g., non-muscle-invasive bladder cancer (NMIBC)), breast cancer, or a solid tumor. In some embodiments, the solid tumor is, for example, a sarcoma, carcinoma, or lymphoma.

In some embodiments, the present invention relates to a method of increasing the safety, increasing the tolerability (e.g., decreasing dysuria, urgency, or malaise), and/or decreasing the likelihood to cause infection in the bloodstream or disseminated bloodstream infection compared to non-recombinant BCG, comprising administering an expression vector, expression cassette, strain, and/or pharmaceutical compositions of the invention described herein to a subject.

### Pharmaceutical Preparations

Pharmaceutical compositions of the present invention comprise an effective amount of one or more inducers of expression of Type 1 interferon such as such as a BCG strain expressing one or more of the following proteins: a RV1354c protein, or functional part thereof; a cyclic GMP-AMP synthase (DncV) protein, or functional part thereof; a cyclic GMP-AMP synthase (cGAS) protein, or functional part thereof; a DNA integrity scanning (disA) protein which functions as a denylate cyclase, or functional part thereof, dissolved or dispersed in a pharmaceutically acceptable carrier. The phrase “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one inducer of expression of Type 1 interferon or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The inducer of expression of Type 1 interferon (such as a BCG strain expressing one or more of the following proteins: a RV1354c protein, or functional part thereof; a cyclic GMP-AMP synthase (DncV) protein, or functional part thereof; a cyclic GMP-AMP synthase (cGAS) protein, or functional part thereof; a DNA integrity scanning (disA) protein which functions as a denylate cyclase, or functional part thereof) may comprise different types of carriers depending on whether it is to be administered in solid, liquid or aerosol form, and whether it need to be sterile for such routes of administration as injection. In some embodiments, the present invention (e.g., expression vectors, strains, or pharmaceutical compositions) can be administered intravenously, intradermally, transdermally, intrathecally, intraarterially, intraperitoneally, intranasally, intravaginally, intrarectally, intravesically (e.g., administered directly into the bladder, e.g., by injection, or by intravesical instillation), intratumorally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18th Ed. Mack Printing Company, 1990, and Francica et al. TNFα and radio-resistant stromal cells are essential for therapeutic efficacy of cyclic dinucleotide STING agonists in non-immunogenic tumors. Cancer Immunol Res. 2018 Feb. 22. PMID: 29472271, incorporated herein by reference).

Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention includes the use of pharmaceutical lipid vehicle compositions that include inducer of expression of Type 1 interferon, one or more lipids, and an aqueous solvent. As used herein, the term “lipid” includes any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the inducer of inducer of expression of Type 1 interferon of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### A. Alimentary Compositions and Formulations

In one embodiment of the present disclosure, the inducers of expression of inducer of expression of Type 1 interferon of the present invention are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz E, Jacob J S, Jong Y S, Carino G P, Chickering D E, Chaturvedi P, Santos C A, Vijayaraghavan K, Montgomery S, Bassett M, Morrell C. Biologically erodable microspheres as potential oral drug delivery systems. Nature. 1997;386:410-4. PMID: 9121559; Hwang M J, Ni X, Waldman M, Ewig C S, Hagler A T. Derivation of class II force fields. VI. Carbohydrate compounds and anomeric effects. Biopolymers. 1998;45:435-68. PMID: 9538697; Hwang J S, Chae S Y, Lee M K, Bae Y H. Synthesis of sulfonylurea conjugated copolymer via PEO spacer and its in vitro short-term bioactivity in insulin secretion from islets of Langerhans. Biomaterials. 1998;19:1189-95. PMID: 9720902; Hwang S J, Park H, Park K. Gastric retentive drug-delivery systems. Crit Rev Ther Drug Carrier Syst. 1998;15:243-84. PMID: 9699081; U.S. Pat. Nos. 5,641,515; 5,580,579; and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, inducer of expression of Type 1 interferon of the present invention may be administered via a parenteral route. As used herein, the term “parenteral” includes routes that bypass the alimentary tract. Specifically, the pharmaceutical compositions disclosed herein may be administered, for example, intravenously, intradermally, intramuscularly, intraarterially, intrathecally, subcutaneous, or intraperitoneally. See, e.g., U.S. Pat. Nos. 6,7537,514; 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose.

Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see, for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other preferred embodiments of the invention, the active compound inducer of expression of Type 1 interferon of the present invention may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga M, Serizawa Y, Azechi Y, Ochiai A, Kosaka Y, Igarashi R, Mizushima Y. Microparticle resins as a potential nasal drug delivery system for insulin. J Control Release. 1998; 52:81-7. PMID: 9685938) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety). The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, an inducer of expression of Type 1 interferon of the present invention (such as a BCG strain expressing one or more of the following proteins: a RV1354c protein, or functional part thereof; a cyclic GMP-AMP synthase (DncV) protein, or functional part thereof; a cyclic GMP-AMP synthase (cGAS) protein, or functional part thereof; a DNA integrity scanning (disA) protein which functions as a denylate cyclase, or functional part thereof) may be comprised in a kit.

The kits may comprise a suitably aliquoted inducer of expression of Type 1 interferon of the present invention and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the inducer of expression of Type 1 interferon of the present invention and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The inducer of expression of Type 1 interferon of the present invention composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The Examples above have been included to provide guidance to one of ordinary skill in the art for practicing representative embodiments of the presently disclosed subject matter. In light of the present disclosure and the general level of skill in the art, those of skill can appreciate that the Examples above are intended to be exemplary only and that numerous changes, modifications, and alterations can be employed without departing from the scope of the presently disclosed subject matter. The Examples above are offered by way of illustration and not by way of limitation.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A vector comprising a nucleic acid sequence expressing a protein or functional part thereof that makes a STING agonist.

2. The vector of claim 1 wherein the STING agonist is selected from the group consisting of 3′-5′ c-di-AMP (also known as c-di-AMP); 3′-5′ c-di-GMP (also known as c-di-GMP); 3′-3′cGAMP ; 2′-3′cGAMP and a combination thereof.

3. The vector of claim 2 comprising the nucleic acid sequence selected from the group consisting of a first nucleic acid sequence encoding a Rv1354c protein, or a functional part thereof; a second nucleic acid sequence encoding a 3′-3′ cyclic GMP-AMP synthase (DncV) protein, or a functional part thereof; a third nucleic acid sequence encoding a 2′-3′ cyclic GMP-AMP synthase (cGAS) protein, or a functional part thereof; a fourth nucleic acid sequence encoding a DNA integrity scanning (disA) protein, or a functional part thereof and a combination thereof.

4. The vector of claim 1 further comprising a fifth nucleic acid sequence encoding a PanC and a PanD protein or functional part thereof.

5. The vector of claim 4 wherein the vector is free of an antibiotic resistance gene.

6. The vector of claim 1 wherein the vector is selected from the group consisting of a vector that stably integrates into the genome of a bacterium, a vector that stably replicates episomally in multiple copies within a bacterium, and/or a combination thereof.

7. The vector of claim 3 further comprising a fifth nucleic acid sequence that encodes a protein or nucleic acid sequence that knocks out the expression of a phosphodiesterase gene or a phosphodiesterase domain of a Mycobacterium.

8. The vector of claim 3 wherein the third nucleic acid sequence overexpresses the cyclase domains of the cyclic GMP-AMP synthase (cGAS) protein.

9. The vector of claim 3 wherein the third nucleic acid sequence expresses a cyclic GMP-AMP synthase (cGAS) protein having a regulatory DNA recognition capability that is non-functional.

10. A strain of Mycobacteria comprising a vector comprising a protein or functional part thereof that makes a STING agonist.

11. The strain of claim 10 wherein the STING agonist is selected from the group consisting of 3′-5′ c-di-AMP (also known as c-di-AMP); 3′-5′ c-di-GMP (also known as c-di-GMP); 3′ -3′cGAMP; 2′ -3′ cGAMP and a combination thereof.

12. The strain of claim 10 wherein the a nucleic acid sequence selected from the group consisting of a first nucleic acid sequence encoding a Rv1354c protein, or a functional part thereof a second nucleic acid sequence encoding a 3′-3′ cyclic GMP-AMP synthase (DncV) protein, or a functional part thereof a third nucleic acid sequence encoding a 2′-3′ cyclic GMP-AMP synthase (cGAS) protein, or a functional part thereof a fourth nucleic acid sequence encoding a DNA integrity scanning (DisA) protein, or a functional part thereof and a combination thereof.

13. The strain of claim 12 wherein the strain of Mycobacteria is Mycobacterium tuberculosis, Mycobacterium bovis, or a combination thereof.

14. The strain of claim 13 wherein the strain of Mycobacteria is Mycobacterium bacillus Calmette Guerin (BCG).

15. The strain of claim 14 wherein the strain is a panthothenate (panCD mutant) auxotroph of BCG and the vector comprises a panCD nucleic acid encoding a PanC protein or a functional part thereof and a nucleic acid sequence encoding a PanD protein or functional part thereof.

16. The strain of claim 16 wherein the strain is free of a genomic antibiotic resistance gene.

17. The strain of claim 16, wherein the vector is free of an antibiotic resistance gene.

18. The strain of claim 10, wherein the vector is selected from a group consisting of a vector that is integrated into the Mycobacterium chromosome, stably replicates episomally in multiple copies with the Mycobacterium, and a combination thereof.

19. The strain of claim 10 wherein the strain is free of an antibiotic resistance gene.

20. A pharmaceutical composition, comprising any one of the strains of Mycobacteria of claims 10, and (ii) a pharmaceutically acceptable carrier.

21. A method of of eliciting a Type 1 interferon response, enhancing the expression of pro-inflammatory cytokine, and/or eliciting trained immunity in subject comprising the steps of:
administering a pharmaceutical composition comprising anyone of the strains strains of Mycobacteria of claims 10, and eliciting a Type 1 interferon response, enhancing the expression of pro-inflammatory cytokine, and/or eliciting trained immunity in the subject.

22. The method of claim 21, wherein the pharmaceutical composition is administered into the bladder of the subject by a catheter.

23. A method of using a strain of Mycobacteria comprising a vector expressing a protein that makes a STING agonist to treat or prevent cancer in a subject comprising the steps of:
administering a pharmaceutical composition comprising a strain of Mycobacteria comprising a vector expressing a protein that makes a STING agonist or a functional part thereof to a subject having cancer; and
treating or preventing cancer in the subject.

24. The method of claim 23 wherein the STING agonist is selected from the group consisting of 3′-5′ c-di-AMP (also known as c-di-AMP); 3′-5′ c-di-GMP (also known as c-diGMP); 3′-3′cGAMP; 2′-3′cGAMP and a combination thereof.

25. The method of claim 24 comprising a nucleic acid sequence selected from the group consisting of a first nucleic acid sequence encoding a Rv1354c protein, or a functional part thereof; a second nucleic acid sequence encoding a 3′-3′cyclic GMP-AMP synthase (DncV) protein, or a functional part thereof; a third nucleic acid sequence encoding a 2′-3′ cyclic GMP-AMP synthase (cGAS) protein, or a functional part thereof; a fourth nucleic acid sequence encoding a DNA integrity scanning (DisA) protein, or a functional part thereof and a combination thereof.

26. The method of claim 23 wherein the cancer is selected from the group consisting of epithelial cancers, breast cancer, non-muscle invasive bladder cancer, and a combination thereof.

27. The method of claim 26 wherein the cancer is non-muscle invasive bladder cancer and is a BCG-unresponsive non-muscle invasive bladder cancer (BCG-unresponsive NMIBC) and the pharmaceutical composition is administered by intravesical instillation.

28. The method of claim 26 wherein the cancer is non-muscle invasive bladder cancer and is a BCG-naïve non-muscle invasive bladder cancer (BCG-naïve NMIBC) and the pharmaceutical composition is administered by intravesical instillation.

29. The method of claim 26 wherein the epithelial cancer is selected from the group consisting of colon cancer, uterine cancer, cervical cancer, vaginal cancer, esophageal cancer, nasopharyngeal cancer, endobronchial cancer, and a combination thereof and the pharmaceutical composition is administered by to a luminal surface of the epithelial cancer.

30. The method of claim 23 wherein the cancer is selected from a solid tumor, liquid tumor.

31. The method of claim 30 wherein the pharmaceutical composition is administered by intratumoral injection.

32. The method of claim 30 wherein the pharmaceutical composition is administered by systemic infusion.

33. The method of claim 23 comprising the step of administering a checkpoint inhibitor.

34. The method of claim 33 wherein the checkpoint inhibitor is selected from the group consisting of ipilimumab (anti-CTLA-4), nivolumab (anti-PD-1), pembrolizumab (anti-PD-1), cemiplimab (anti-PD-1), atezolizumab (anti-PD-L1), avelumab (anti-PD-L1), durvalumab (anti-PD-L1) and a combination thereof.

35. The method of claim 23 wherein the cancer is bladder cancer and a catheter administers the pharmaceutical composition.
